SEQUENCE LISTING (123 sequences)

SEQ ID NO: 1 - protein, 5 aa, Mus Musculus
SYGIT

SEQ ID NO: 2 - protein, 17 aa, Mus Musculus
ENYPRSGNTYYNEKFKG

SEQ ID NO: 3 - protein, 17 aa, Mus Musculus
CEFISTVVAPYYYALDY

SEQ ID NO: 4 - protein, 17 aa, Artificial Sequence
  Mutated CDR
SEFISTVVAPYYYALDY

SEQ ID NO: 5 - protein, 16 aa, Mus Musculus
KASKKVTIFGSISALH

SEQ ID NO: 6 - protein, 7 aa, Mus Musculus
NGAKLES

SEQ ID NO: 7 - protein, 9 aa, mus musculus
LQNKEVPYT

SEQ ID NO: 8 - protein, 126 aa, mus musculus
QVQLQQSGAELARPGTSVKLSCKASGYTFTSYGITWVKQRTGQGLEWIGENYPRSGNTYYNEKFKGKATLTADKSSSTAYMELRSLTSEDSAVYFCARCEFISTVVAPYYYALDYWGQGTSVTVSS

SEQ ID NO: 9 - DNA, 378 bp, mus musculus
caggttcagctgcagcagtctggagctgagctggcgaggcctgggacttcagtgaagctgtcctgcaaggcttctggctacaccttcacaagctatggtataacctgggtgaagcagagaactggacagggccttgagtggattggagagaattatcctagaagtggtaatacttactacaatgagaaattcaagggcaaggccacactgactgcagacaaatcctccagcacagcgtacatggagctccgcagcctgacatctgaggactctgcggtctatttctgtgcaagatgcgaatttattagtacggtagtagctccctattactatgctctggactactggggtcaaggaacctcagtcaccgtctcctca

SEQ ID NO: 10 - protein, 119 aa, mus musculus
DIVLTQSPASLAVSLGQKATISCKASKKVTIFGSISALHWYQQKPGQPPKLIYNGAKLESGVSARFSDSGSQNRSPFGNQLSFTLTIDPVEADDAATYYCLQNKEVPYTFGGGTKLEIK

SEQ ID NO: 11 - DNA, 357 bp, mus musculus
gacattgtactaacccaatctccagcatctttggctgtgtctctagggcagaaggccaccatctcctgcaaggccagcaaaaaagtcactatatttggctctataagtgctctgcactggtaccaacagaaaccaggacagccacccaaactcatctataatggagccaaactagaatctggggtcagtgccaggttcagtgacagtgggtctcagaaccgctcaccatttggaaatcagctcagcttcaccctcaccattgatcctgtggaggctgatgatgcagcaacctattactgtctgcaaaataaagaggttccgtacacgttcggaggggggaccaagctggaaataaaa

SEQ ID NO: 12 - protein, 457 aa, Artificial Sequence
  Chimeric sequence
QVQLQQSGAELARPGTSVKLSCKASGYTFTSYGITWVKQRTGQGLEWIGENYPRSGNTYYNEKFKGKATLTADKSSSTAYMELRSLTSEDSAVYFCARCEFISTVVAPYYYALDYWGQGTSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1371 bp, Artificial Sequence
  Chimeric sequence
caggttcagctgcagcagtctggagctgagctggcgaggcctgggacttcagtgaagctgtcctgcaaggcttctggctacaccttcacaagctatggtataacctgggtgaagcagagaactggacagggccttgagtggattggagagaattatcctagaagtggtaatacttactacaatgagaaattcaagggcaaggccacactgactgcagacaaatcctccagcacagcgtacatggagctccgcagcctgacatctgaggactctgcggtctatttctgtgcaagatgcgaatttattagtacggtagtagctccctattactatgctctggactactggggtcaaggaacctcactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 14 - protein, 226 aa, Artificial Sequence
  Chimeric sequence
DIVLTQSPASLAVSLGQKATISCKASKKVTIFGSISALHWYQQKPGQPPKLIYNGAKLESGVSARFSDSGSQNRSPFGNQLSFTLTIDPVEADDAATYYCLQNKEVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 678 bp, Artificial Sequence
  Chimeric sequence
gacattgtactaacccaatctccagcatctttggctgtgtctctagggcagaaggccaccatctcctgcaaggccagcaaaaaagtcactatatttggctctataagtgctctgcactggtaccaacagaaaccaggacagccacccaaactcatctataatggagccaaactagaatctggggtcagtgccaggttcagtgacagtgggtctcagaaccgctcaccatttggaaatcagctcagcttcaccctcaccattgatcctgtggaggctgatgatgcagcaacctattactgtctgcaaaataaagaggttccgtacacgttcggaggggggaccaagctggaaataaaacgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 16 - protein, 126 aa, Artificial Sequence
  Humanised sequence
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARSEFISTVVAPYYYALDYWGQGTLVTVSS

SEQ ID NO: 17 - DNA, 378 bp, Artificial Sequence
  Humanised sequence
caggtgcagctggtgcagagcggcgccgaagtgaagaagcccggctccagcgtgaaggtgagctgcaaagcctcaggctacaccttcaccagctacggcatcacttgggtgaggcaggcccccggccagggactggagtggatgggagagaactaccccaggagcggcaacacctactacaacgagaagttcaagggcagggtgaccatcaccgccgacaagagcaccagcaccgcctacatggagctgagcagcctgaggagcgaggacaccgctgtgtactactgcgccaggagcgagttcatcagcaccgtcgtggccccctactactacgccctcgactattggggccagggcacactagtgaccgtgtccagc

SEQ ID NO: 18 - protein, 111 aa, Artificial Sequence
  Humanised sequence
DIVMTQSPDSLAVSLGERATINCKASKKVTIFGSISALHWYQQKPGQPPKLIYNGAKLESGVPDRFSGSGSGTDFTLTISSLQAEDVAVYYCLQNKEVPYTFGGGTKVEIK

SEQ ID NO: 19 - protein, 333 aa, Artificial Sequence
  Humanised sequence
GACATCGTGATGACCCAGAGCCCCGATAGCCTCGCTGTGAGCCTGGGCGAGAGGGCCACCATCAACTGCAAGGCCAGCAAGAAGGTCACCATCTTCGGCAGCATCTCCGCCCTGCACTGGTACCAGCAGAAGCCCGGACAGCCCCCCAAGCTGATCTACAACGGCGCCAAGCTGGAGAGCGGCGTGCCCGACAGGTTTAGCGGCAGCGGCAGCGGCACAGACTTCACCCTGACCATTAGCAGCCTGCAGGCCGAAGACGTGGCCGTGTACTACTGCCTGCAGAACAAGGAGGTGCCCTACACCTTCGGCGGGGGCACCAAAGTGGAGATCAAG

SEQ ID NO: 20 - protein, 119 aa, Artificial Sequence
  Humanised sequence
DIVMTQSPDSLAVSLGERATINCKASKKVTIFGSISALHWYQQKPGQPPKLIYNGAKLESGVSDRFSDSGSQNRSPFGNQLSFTLTISSLQAEDVAVYYCLQNKEVPYTFGGGTKVEIK

SEQ ID NO: 21 - DNA, 357 bp, Artificial Sequence
  Humanised sequence
gacatcgtgatgactcagtctcccgacagcctggccgtgagcctgggcgagagggccaccatcaactgcaaggccagcaagaaggtgaccatcttcgggagcatctccgccctgcactggtatcagcagaaacccggacagccccccaagctgatctacaacggcgccaagctggaaagcggcgtgagcgacaggttcagcgatagcggcagccagaacaggagccctttcggcaaccagctgagcttcaccctgaccatcagcagcctccaggccgaggacgtcgcagtgtactactgcctgcagaacaaggaggtgccctacacctttggcggcggcaccaaggtggagattaag

SEQ ID NO: 22 - protein, 119 aa, Artificial Sequence
  Humanised sequence
DIVMTQTPLSLSVTPGQPASISCKASKKVTIFGSISALHWYLQKPGQPPQLIYNGAKLESGVSDRFSDSGSQNRSPFGNQLSFTLKISRVEAEDVGVYYCLQNKEVPYTFGGGTKVEIK

SEQ ID NO: 23 - DNA, 357 bp, Artificial Sequence
  Humanised sequence
gatatcgtgatgacccagacccccctgagcctgagcgtgactccaggccagcccgccagcatcagctgcaaggccagcaagaaggtgaccatcttcggcagcattagcgccctccactggtacctgcagaaacccgggcagcccccccagctgatctataacggcgctaagctggagagcggcgtgtccgacaggttcagcgactctggaagccagaacaggagccccttcggcaaccagctgagcttcaccctgaagatcagcagggtggaagccgaggacgtgggcgtgtactactgcctgcagaacaaggaggtgccctacaccttcggaggcggcaccaaggtcgagatcaag

SEQ ID NO: 24 - protein, 111 aa, Artificial Sequence
  Humanised sequence
DIVMTQTPLSLSVTPGQPASISCKASKKVTIFGSISALHWYLQKPGQPPQLIYNGAKLESGVSDRFSDSGSGTDFTLKISRVEAEDVGVYYCLQNKEVPYTFGGGTKVEIK

SEQ ID NO: 25 - DNA, 333 bp, Artificial Sequence
  Humanised sequence
gacatcgtgatgacccagactcccctgtccctgagcgtgacccccggacagcccgccagcatcagctgcaaggccagcaagaaggtgaccatcttcggcagcatcagcgccctgcactggtacctccagaagcccgggcagcccccacagctgatctacaacggcgccaagctggagagcggcgtgagcgacaggttctctgatagcggcagcggcaccgacttcaccctgaagattagcagggtggaggccgaggacgtgggcgtgtactactgcctgcagaacaaggaggtgccctacaccttcggcggcggcaccaaagtcgagatcaag

SEQ ID NO: 26 - protein, 456 aa, Artificial Sequence
  Humanised sequence
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARSEFISTVVAPYYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1368 bp, Artificial Sequence
  Humanised sequence
caggtgcagctggtgcagagcggcgccgaagtgaagaagcccggctccagcgtgaaggtgagctgcaaagcctcaggctacaccttcaccagctacggcatcacttgggtgaggcaggcccccggccagggactggagtggatgggagagaactaccccaggagcggcaacacctactacaacgagaagttcaagggcagggtgaccatcaccgccgacaagagcaccagcaccgcctacatggagctgagcagcctgaggagcgaggacaccgctgtgtactactgcgccaggagcgagttcatcagcaccgtcgtggccccctactactacgccctcgactattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 28 - protein, 218 aa, Artificial Sequence
  Humanised sequence
DIVMTQSPDSLAVSLGERATINCKASKKVTIFGSISALHWYQQKPGQPPKLIYNGAKLESGVPDRFSGSGSGTDFTLTISSLQAEDVAVYYCLQNKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 654 bp, Artificial Sequence
  Humanised sequence
gacatcgtgatgacccagagccccgatagcctcgctgtgagcctgggcgagagggccaccatcaactgcaaggccagcaagaaggtcaccatcttcggcagcatctccgccctgcactggtaccagcagaagcccggacagccccccaagctgatctacaacggcgccaagctggagagcggcgtgcccgacaggtttagcggcagcggcagcggcacagacttcaccctgaccattagcagcctgcaggccgaagacgtggccgtgtactactgcctgcagaacaaggaggtgccctacaccttcggcgggggcaccaaagtggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 30 - protein, 226 aa, Artificial Sequence
  Humanised sequence
DIVMTQSPDSLAVSLGERATINCKASKKVTIFGSISALHWYQQKPGQPPKLIYNGAKLESGVSDRFSDSGSQNRSPFGNQLSFTLTISSLQAEDVAVYYCLQNKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 678 bp, Artificial Sequence
  Humanised sequence
gacatcgtgatgactcagtctcccgacagcctggccgtgagcctgggcgagagggccaccatcaactgcaaggccagcaagaaggtgaccatcttcgggagcatctccgccctgcactggtatcagcagaaacccggacagccccccaagctgatctacaacggcgccaagctggaaagcggcgtgagcgacaggttcagcgatagcggcagccagaacaggagccctttcggcaaccagctgagcttcaccctgaccatcagcagcctccaggccgaggacgtcgcagtgtactactgcctgcagaacaaggaggtgccctacacctttggcggcggcaccaaggtggagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 32 - protein, 226 aa, Artificial Sequence
  Humanised sequence
DIVMTQTPLSLSVTPGQPASISCKASKKVTIFGSISALHWYLQKPGQPPQLIYNGAKLESGVSDRFSDSGSQNRSPFGNQLSFTLKISRVEAEDVGVYYCLQNKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - DNA, 678 bp, Artificial Sequence
  Humanised sequence
gatatcgtgatgacccagacccccctgagcctgagcgtgactccaggccagcccgccagcatcagctgcaaggccagcaagaaggtgaccatcttcggcagcattagcgccctccactggtacctgcagaaacccgggcagcccccccagctgatctataacggcgctaagctggagagcggcgtgtccgacaggttcagcgactctggaagccagaacaggagccccttcggcaaccagctgagcttcaccctgaagatcagcagggtggaagccgaggacgtgggcgtgtactactgcctgcagaacaaggaggtgccctacaccttcggaggcggcaccaaggtcgagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 34 - protein, 218 aa, Artificial Sequence
  Humanised sequence
DIVMTQTPLSLSVTPGQPASISCKASKKVTIFGSISALHWYLQKPGQPPQLIYNGAKLESGVSDRFSDSGSGTDFTLKISRVEAEDVGVYYCLQNKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 654 bp, Artificial Sequence
  Humanised sequence
gacatcgtgatgacccagactcccctgtccctgagcgtgacccccggacagcccgccagcatcagctgcaaggccagcaagaaggtgaccatcttcggcagcatcagcgccctgcactggtacctccagaagcccgggcagcccccacagctgatctacaacggcgccaagctggagagcggcgtgagcgacaggttctctgatagcggcagcggcaccgacttcaccctgaagattagcagggtggaggccgaggacgtgggcgtgtactactgcctgcagaacaaggaggtgccctacaccttcggcggcggcaccaaagtcgagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 36 - protein, 19 aa, Homo Sapiens
MGWSCIILFLVATATGVHS

SEQ ID NO: 37 - protein, 189 aa, Homo Sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 38 - DNA, 567 bp, Homo Sapiens
atgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccc

SEQ ID NO: 39 - protein, 328 aa, Homo Sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 40 - DNA, 984 bp, Homo Sapiens
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 41 - protein, 253 aa, Homo Sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 42 - DNA, 759 bp, Homo Sapiens
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcc

SEQ ID NO: 43 - protein, 189 aa, Macaca fascicularis
MLGSRAVMLLLLLSWTAQGRAVPGGSSPAWAQCQQLSQKLCTLAWSAHPLVGHMDLREEGDEETTNDVPHIQCGDGCDPQGLRDNSQFCLQRIRQGLIFYEKLLGSDIFTGEPSLLPDSPVGQLHASLLGLSQLLQPEGHHWETQQIPSPSPSQPWQRLLLRFKILRSLQAFVAVAARVFAHGAATLSP

SEQ ID NO: 44 - DNA, 567 bp, Macaca fascicularis
atgctggggagcagagctgtaatgctgctgttgctgctgtcctggacagctcagggcagggctgtgcctgggggcagcagccctgcctgggctcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggattcgccagggtctgattttttacgagaagctactgggatcggatattttcacaggggagccttctctgctgcctgatagccctgtgggccagcttcatgcctccctactgggcctcagccaactcctgcagcctgagggtcaccactgggagactcagcagattccaagccccagtcccagccagccatggcagcgcctccttctccgcttcaaaatccttcgcagcctccaggcctttgtggctgtagctgcccgggtctttgcccatggagcagcaaccctgagtccc

SEQ ID NO: 45 - protein, 328 aa, Macaca fascicularis
MCHQQLVISWFSLVFLASPLMAIWELKKDVYVVELDWYPDAPGEMVVLTCDTPEEDGITWTLDQSGEVLGSGKTLTIQVKEFGDAGQYTCHKGGEALSHSLLLLHKKEDGIWSTDVLKDQKEPKNKTFLRCEAKNYSGRFTCWWLTTISTDLTFSVKSSRGSSNPQGVTCGAVTLSAERVRGDNKEYEYSVECQEDSACPAAEERLPIEVMVDAIHKLKYENYTSSFFIRDIIKPDPPKNLQLKPLKNSRQVEVSWEYPDTWSTPHSYFSLTFCIQVQGKSKREKKDRIFTDKTSATVICRKNASFSVQAQDRYYSSSWSEWASVPCS

SEQ ID NO: 46 - DNA, 984 bp, Macaca fascicularis
atgtgtcaccagcagctggtcatctcttggttttccctggtttttctggcatctcccctcatggccatatgggaactgaagaaagacgtttatgttgtagaattggactggtacccggatgcccctggagaaatggtggtcctcacctgtgacacccctgaagaagatggtatcacctggaccttggaccagagtggtgaggtcttaggctctggcaaaaccctgaccatccaagtcaaagagtttggagatgctggccagtacacctgtcacaaaggaggcgaggctctaagccattcactcctgctgcttcacaaaaaggaagatggaatttggtccactgatgttttaaaggaccagaaagaacccaaaaataagacctttctaagatgcgaggccaaaaattattctggacgtttcacctgctggtggctgacgacaatcagtactgatctgacattcagtgtcaaaagcagcagaggctcttctaacccccaaggggtgacgtgtggagccgttacactctctgcagagagggtcagaggggacaataaggagtatgagtactcagtggagtgccaggaggacagtgcctgcccagccgctgaggagaggctgcccattgaggtcatggtggatgccattcacaagctcaagtatgaaaactacaccagcagcttcttcatcagggacatcatcaaacccgacccacccaagaacttgcagctgaagccattaaagaattctcggcaggtggaggtcagctgggagtaccctgacacctggagtactccacattcctacttctccctgacattctgcatccaggtccagggcaagagcaagagagaaaagaaagatagaatcttcacagacaagacctcagccacggtcatctgccgcaaaaatgccagctttagcgtgcaggcccaggaccgctactatagctcatcttggagcgaatgggcatctgtgccctgcagt

SEQ ID NO: 47 - protein, 629 aa, Homo Sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 48 - protein, 126 aa, Artificial Sequence
  Humanised sequence
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARAEFISTVVAPYYYALDYWGQGTLVTVSS

SEQ ID NO: 49 - DNA, 378 bp, Artificial Sequence
  Humanised sequence
caggtgcagctggtgcagagcggcgccgaagtgaagaagcccggctccagcgtgaaggtgagctgcaaagcctcaggctacaccttcaccagctacggcatcacttgggtgaggcaggcccccggccagggactggagtggatgggagagaactaccccaggagcggcaacacctactacaacgagaagttcaagggcagggtgaccatcaccgccgacaagagcaccagcaccgcctacatggagctgagcagcctgaggagcgaggacaccgctgtgtactactgcgccagggctgagttcatcagcaccgtcgtggccccctactactacgccctcgactattggggccagggcacactagtgaccgtgtccagc

SEQ ID NO: 50 - protein, 126 aa, Artificial Sequence
  Humanised sequence
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARVEFISTVVAPYYYALDYWGQGTLVTVSS

SEQ ID NO: 51 - DNA, 378 bp, Artificial Sequence
  Humanised sequence
caggtgcagctggtgcagagcggcgccgaagtgaagaagcccggctccagcgtgaaggtgagctgcaaagcctcaggctacaccttcaccagctacggcatcacttgggtgaggcaggcccccggccagggactggagtggatgggagagaactaccccaggagcggcaacacctactacaacgagaagttcaagggcagggtgaccatcaccgccgacaagagcaccagcaccgcctacatggagctgagcagcctgaggagcgaggacaccgctgtgtactactgcgccagggtggagttcatcagcaccgtcgtggccccctactactacgccctcgactattggggccagggcacactagtgaccgtgtccagc

SEQ ID NO: 52 - protein, 126 aa, Artificial Sequence
  Humanised sequence
QVQLVQSGAEVKKPGSSVKVSCKASGYTFTSYGITWVRQAPGQGLEWMGEDYPRSGNTYYNEKFKGRVTITADKSTSTAYMELSSLRSEDTAVYYCARSEFISTVVAPYYYALDYWGQGTLVTVSS

SEQ ID NO: 53 - DNA, 378 bp, Artificial Sequence
  Humanised sequence
caggtgcagctggtgcagagcggcgccgaagtgaagaagcccggctccagcgtgaaggtgagctgcaaagcctcaggctacaccttcaccagctacggcatcacttgggtgaggcaggcccccggccagggactggagtggatgggagaggactaccccaggagcggcaacacctactacaacgagaagttcaagggcagggtgaccatcaccgccgacaagagcaccagcaccgcctacatggagctgagcagcctgaggagcgaggacaccgctgtgtactactgcgccaggagcgagttcatcagcaccgtcgtggccccctactactacgccctcgactattggggccagggcacactagtgaccgtgtccagc

SEQ ID NO: 54 - protein, 126 aa, Artificial Sequence
  Humanised sequence
QVQLVQSGAEVKKPGSSVKVSCKASGYTFASYGITWVRQAPGQGLEWMGENYPRSGNTYYNEKFKGRVTITADKSTSTAYMELSSLRSEDTAMYYCARSEFISTVVAPYYYALDYWGQGTLVTVSS

SEQ ID NO: 55 - DNA, 378 bp, Artificial Sequence
  Humanised sequence
caggtgcagctggtgcagagcggcgccgaagtgaagaagcccggctccagcgtgaaggtgagctgcaaagcctcaggctacaccttcgccagctacggcatcacttgggtgaggcaggcccccggccagggactggagtggatgggagagaactaccccaggagcggcaacacctactacaacgagaagttcaagggcagggtgaccatcaccgccgacaagagcaccagcaccgcctacatggagctgagcagcctgaggagcgaggacaccgctatgtactactgcgccaggagcgagttcatcagcaccgtcgtggccccctactactacgccctcgactattggggccagggcacactagtgaccgtgtccagc

SEQ ID NO: 56 - protein, 111 aa, Artificial Sequence
  Humanised sequence
DIVMTQSPDSLAVSLGERATINCKASKKVTIFGSTSALHWYQQKPGQPPKLIYNGAKLESGVPDRFSGSGSGTDFTLTISSLQAEDVAVYYCLQNKEVPYTFGGGTKVEIK

SEQ ID NO: 57 - DNA, 333 bp, Artificial Sequence
  Humanised sequence
gacatcgtgatgacccagagccccgatagcctcgctgtgagcctgggcgagagggccaccatcaactgcaaggccagcaagaaggtcaccatcttcggcagcacctccgccctgcactggtaccagcagaagcccggacagccccccaagctgatctacaacggcgccaagctggagagcggcgtgcccgacaggtttagcggcagcggcagcggcacagacttcaccctgaccattagcagcctgcaggccgaagacgtggccgtgtactactgcctgcagaacaaggaggtgccctacaccttcggcgggggcaccaaagtggagatcaag

SEQ ID NO: 58 - protein, 111 aa, Artificial Sequence
  Humanised sequence
DIVMTQSPDSLAVSLGERATINCKASKKVTIFGSTSALHWYQQKPGQPPKLIYNGAKPESGVPDRFSGSGSGTDFTLTISSLQAEDVAVYYCLQNKEVPYTFGGGTKVEIK

SEQ ID NO: 59 - DNA, 333 bp, Artificial Sequence
  Humanised sequence
gacatcgtgatgacccagagccccgatagcctcgctgtgagcctgggcgagagggccaccatcaactgcaaggccagcaagaaggtcaccatcttcggcagcacctccgccctgcactggtaccagcagaagcccggacagccccccaagctgatctacaacggcgccaagcccgagagcggcgtgcccgacaggtttagcggcagcggcagcggcacagacttcaccctgaccattagcagcctgcaggccgaagacgtggccgtgtactactgcctgcagaacaaggaggtgccctacaccttcggcgggggcaccaaagtggagatcaag

SEQ ID NO: 60 - protein, 456 aa, Artificial Sequence
  Humanised sequence
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARAEFISTVVAPYYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - DNA, 1368 bp, Artificial Sequence
  Humanised sequence
caggtgcagctggtgcagagcggcgccgaagtgaagaagcccggctccagcgtgaaggtgagctgcaaagcctcaggctacaccttcaccagctacggcatcacttgggtgaggcaggcccccggccagggactggagtggatgggagagaactaccccaggagcggcaacacctactacaacgagaagttcaagggcagggtgaccatcaccgccgacaagagcaccagcaccgcctacatggagctgagcagcctgaggagcgaggacaccgctgtgtactactgcgccagggctgagttcatcagcaccgtcgtggccccctactactacgccctcgactattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 62 - protein, 456 aa, Artificial Sequence
  Humanised sequence
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARVEFISTVVAPYYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 1368 bp, Artificial Sequence
  Humanised sequence
caggtgcagctggtgcagagcggcgccgaagtgaagaagcccggctccagcgtgaaggtgagctgcaaagcctcaggctacaccttcaccagctacggcatcacttgggtgaggcaggcccccggccagggactggagtggatgggagagaactaccccaggagcggcaacacctactacaacgagaagttcaagggcagggtgaccatcaccgccgacaagagcaccagcaccgcctacatggagctgagcagcctgaggagcgaggacaccgctgtgtactactgcgccagggtggagttcatcagcaccgtcgtggccccctactactacgccctcgactattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 64 - protein, 456 aa, Artificial Sequence
  Humanised sequence
QVQLVQSGAEVKKPGSSVKVSCKASGYTFTSYGITWVRQAPGQGLEWMGEDYPRSGNTYYNEKFKGRVTITADKSTSTAYMELSSLRSEDTAVYYCARSEFISTVVAPYYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1368 bp, Artificial Sequence
  Humanised sequence
caggtgcagctggtgcagagcggcgccgaagtgaagaagcccggctccagcgtgaaggtgagctgcaaagcctcaggctacaccttcaccagctacggcatcacttgggtgaggcaggcccccggccagggactggagtggatgggagaggactaccccaggagcggcaacacctactacaacgagaagttcaagggcagggtgaccatcaccgccgacaagagcaccagcaccgcctacatggagctgagcagcctgaggagcgaggacaccgctgtgtactactgcgccaggagcgagttcatcagcaccgtcgtggccccctactactacgccctcgactattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 66 - protein, 456 aa, Artificial Sequence
  Humanised sequence
QVQLVQSGAEVKKPGSSVKVSCKASGYTFASYGITWVRQAPGQGLEWMGENYPRSGNTYYNEKFKGRVTITADKSTSTAYMELSSLRSEDTAMYYCARSEFISTVVAPYYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - DNA, 1368 bp, Artificial Sequence
  Humanised sequence
caggtgcagctggtgcagagcggcgccgaagtgaagaagcccggctccagcgtgaaggtgagctgcaaagcctcaggctacaccttcgccagctacggcatcacttgggtgaggcaggcccccggccagggactggagtggatgggagagaactaccccaggagcggcaacacctactacaacgagaagttcaagggcagggtgaccatcaccgccgacaagagcaccagcaccgcctacatggagctgagcagcctgaggagcgaggacaccgctatgtactactgcgccaggagcgagttcatcagcaccgtcgtggccccctactactacgccctcgactattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 68 - protein, 218 aa, Artificial Sequence
  Humanised sequence
DIVMTQSPDSLAVSLGERATINCKASKKVTIFGSTSALHWYQQKPGQPPKLIYNGAKLESGVPDRFSGSGSGTDFTLTISSLQAEDVAVYYCLQNKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - DNA, 654 bp, Artificial Sequence
  Humanised sequence
gacatcgtgatgacccagagccccgatagcctcgctgtgagcctgggcgagagggccaccatcaactgcaaggccagcaagaaggtcaccatcttcggcagcacctccgccctgcactggtaccagcagaagcccggacagccccccaagctgatctacaacggcgccaagctggagagcggcgtgcccgacaggtttagcggcagcggcagcggcacagacttcaccctgaccattagcagcctgcaggccgaagacgtggccgtgtactactgcctgcagaacaaggaggtgccctacaccttcggcgggggcaccaaagtggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 70 - protein, 218 aa, Artificial Sequence
  Humanised sequence
DIVMTQSPDSLAVSLGERATINCKASKKVTIFGSTSALHWYQQKPGQPPKLIYNGAKPESGVPDRFSGSGSGTDFTLTISSLQAEDVAVYYCLQNKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 654 bp, Artificial Sequence
  Humanised sequence
gacatcgtgatgacccagagccccgatagcctcgctgtgagcctgggcgagagggccaccatcaactgcaaggccagcaagaaggtcaccatcttcggcagcacctccgccctgcactggtaccagcagaagcccggacagccccccaagctgatctacaacggcgccaagcccgagagcggcgtgcccgacaggtttagcggcagcggcagcggcacagacttcaccctgaccattagcagcctgcaggccgaagacgtggccgtgtactactgcctgcagaacaaggaggtgccctacaccttcggcgggggcaccaaagtggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 72 - protein, 17 aa, Artificial Sequence
  mutated CDR
EDYPRSGNTYYNEKFKG

SEQ ID NO: 73 - protein, 17 aa, Artificial Sequence
  mutated CDR
AEFISTVVAPYYYALDY

SEQ ID NO: 74 - protein, 17 aa, Artificial Sequence
  mutated CDR
VEFISTVVAPYYYALDY

SEQ ID NO: 75 - protein, 16 aa, Artificial Sequence
  mutated CDR
KASKKVTIFGSTSALH

SEQ ID NO: 76 - protein, 7 aa, Artificial Sequence
  mutated CDR
NGAKPES

SEQ ID NO: 77 - protein, 7 aa, Artificial Sequence
  mutated CDR
DGAKLES

SEQ ID NO: 78 - protein, 7 aa, Artificial Sequence
  mutated CDR
QGAKLES

SEQ ID NO: 79 - protein, 7 aa, Artificial Sequence
  mutated CDR
DGAKPES

SEQ ID NO: 80 - protein, 7 aa, Artificial Sequence
  mutated CDR
QGAKPES

SEQ ID NO: 81 - protein, 126 aa, Artificial Sequence
  Humanised sequence
QVQLVQSGAEVKKPGSSVKVSCKASGYTFTSYGITWVRQAPGQGLEWMGEDYPRSGNTYYNEKFKGRVTITADKSTSTAYMELSSLRSEDTAVYYCARAEFISTVVAPYYYALDYWGQGTLVTVSS

SEQ ID NO: 82 - protein, 126 aa, Artificial Sequence
  Humanised sequence
QVQLVQSGAEVKKPGSSVKVSCKASGYTFTSYGITWVRQAPGQGLEWMGEDYPRSGNTYYNEKFKGRVTITADKSTSTAYMELSSLRSEDTAVYYCARVEFISTVVAPYYYALDYWGQGTLVTVSS

SEQ ID NO: 83 - protein, 126 aa, Artificial Sequence
  Humanised sequence
QVQLVQSGAEVKKPGSSVKVSCKASGYTFASYGITWVRQAPGQGLEWMGENYPRSGNTYYNEKFKGRVTITADKSTSTAYMELSSLRSEDTAMYYCARAEFISTVVAPYYYALDYWGQGTLVTVSS

SEQ ID NO: 84 - protein, 126 aa, Artificial Sequence
  Humanised sequence
QVQLVQSGAEVKKPGSSVKVSCKASGYTFASYGITWVRQAPGQGLEWMGENYPRSGNTYYNEKFKGRVTITADKSTSTAYMELSSLRSEDTAMYYCARVEFISTVVAPYYYALDYWGQGTLVTVSS

SEQ ID NO: 85 - protein, 126 aa, Artificial Sequence
  Humanised sequence
QVQLVQSGAEVKKPGSSVKVSCKASGYTFASYGITWVRQAPGQGLEWMGENYPRSGNTYYNEKFKGRVTITADKSTSTAYMELSSLRSEDTAVYYCARSEFISTVVAPYYYALDYWGQGTLVTVSS

SEQ ID NO: 86 - protein, 126 aa, Artificial Sequence
  Humanised sequence
QVQLVQSGAEVKKPGSSVKVSCKASGYTFASYGITWVRQAPGQGLEWMGENYPRSGNTYYNEKFKGRVTITADKSTSTAYMELSSLRSEDTAVYYCARAEFISTVVAPYYYALDYWGQGTLVTVSS

SEQ ID NO: 87 - protein, 126 aa, Artificial Sequence
  Humanised sequence
QVQLVQSGAEVKKPGSSVKVSCKASGYTFASYGITWVRQAPGQGLEWMGENYPRSGNTYYNEKFKGRVTITADKSTSTAYMELSSLRSEDTAVYYCARVEFISTVVAPYYYALDYWGQGTLVTVSS

SEQ ID NO: 88 - protein, 126 aa, Artificial Sequence
  Humanised sequence
QVQLVQSGAEVKKPGSSVKVSCKASGYTFASYGITWVRQAPGQGLEWMGEDYPRSGNTYYNEKFKGRVTITADKSTSTAYMELSSLRSEDTAVYYCARSEFISTVVAPYYYALDYWGQGTLVTVSS

SEQ ID NO: 89 - protein, 126 aa, Artificial Sequence
  Humanised sequence
QVQLVQSGAEVKKPGSSVKVSCKASGYTFASYGITWVRQAPGQGLEWMGEDYPRSGNTYYNEKFKGRVTITADKSTSTAYMELSSLRSEDTAVYYCARAEFISTVVAPYYYALDYWGQGTLVTVSS

SEQ ID NO: 90 - protein, 126 aa, Artificial Sequence
  Humanised sequence
QVQLVQSGAEVKKPGSSVKVSCKASGYTFASYGITWVRQAPGQGLEWMGEDYPRSGNTYYNEKFKGRVTITADKSTSTAYMELSSLRSEDTAVYYCARVEFISTVVAPYYYALDYWGQGTLVTVSS

SEQ ID NO: 91 - protein, 107 aa, Homo Sapiens [sequence omitted]

SEQ ID NO: 92 - protein, 330 aa, Homo Sapiens [sequence omitted]

SEQ ID NO: 93 - protein, 218 aa, Artificial Sequence
  Humanised sequence
DIVMTQSPDSLAVSLGERATINCKASKKVTIFGSISALHWYQQKPGQPPKLIYDGAKLESGVPDRFSGSGSGTDFTLTISSLQAEDVAVYYCLQNKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 218 aa, Artificial Sequence
  Humanised sequence
DIVMTQSPDSLAVSLGERATINCKASKKVTIFGSISALHWYQQKPGQPPKLIYQGAKLESGVPDRFSGSGSGTDFTLTISSLQAEDVAVYYCLQNKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 17 aa, Artificial Sequence
  Mutated CDR
SEFISTVMAPYYYALDY

SEQ ID NO: 96 - protein, 111 aa, Artificial Sequence
  Humanised sequence
DIVMTQSPDSLAVSLGERATINCKASKKVTIFGSISALHWYQQKPGQPPKLIYDGAKLESGVPDRFSGSGSGTDFTLTISSLQAEDVAVYYCLQNKEVPYTFGGGTKVEIK

SEQ ID NO: 97 - protein, 111 aa, Artificial Sequence
  Humanised sequence
DIVMTQSPDSLAVSLGERATINCKASKKVTIFGSISALHWYQQKPGQPPKLIYQGAKLESGVPDRFSGSGSGTDFTLTISSLQAEDVAVYYCLQNKEVPYTFGGGTKVEIK

SEQ ID NO: 98 - protein, 17 aa, Artificial Sequence
  Mutated CDR
ENYPRSGNIYYNEKFKG

SEQ ID NO: 99 - protein, 17 aa, Artificial Sequence
  Mutated CDR
ENYPRSGNTYYNEKFRG

SEQ ID NO: 100 - protein, 17 aa, Artificial Sequence
  Mutated CDR
SEFTSTVVAPYYYALDY

SEQ ID NO: 101 - protein, 16 aa, Artificial Sequence
  Mutated CDR
KASKKVTIYGSTSALH

SEQ ID NO: 102 - protein, 7 aa, Artificial Sequence
  Mutated CDR
NSAKLES

SEQ ID NO: 103 - protein, 126 aa, Artificial Sequence
  Humanised sequence
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARSEFISTVMAPYYYALDYWGQGTLVTVSS

SEQ ID NO: 104 - protein, 126 aa, Artificial Sequence
  Humanised sequence
QVQLVQSGAEVKKPGSSVKVSCKASGYTFTSYGITWVRQAPGQGLEWMGEDYPRSGNTYYNEKFKGRVTITADKSTSTAYMELSGLRSEDTAVYYCARSEFISTVVAPYYYALDYWGQGTLVTVSS

SEQ ID NO: 105 - protein, 126 aa, Artificial Sequence
  Humanised sequence
QVQLVQSGAEVKKPGSSVRVSCKASGYTFTSYGITWVRQAPGQGLEWMGENYPRSGNTYYNEKFKGRVTITADKSTSTAYMELSSLRSEDTAVYYCARSEFISTVVAPYYYALDYWGQGTLVTVSS

SEQ ID NO: 106 - protein, 126 aa, Artificial Sequence
  Humanised sequence
QVQLVQSGAEVKKPGSSVKVSCKASGYTFASYGITWVRQAPGQGLEWMGENYPRSGNTYYNEKFKGRVTITADKSTSTAYMELSSLRSEDTAAYYCARSEFISTVVAPYYYALDYWGQGTLVTVSS

SEQ ID NO: 107 - protein, 126 aa, Artificial Sequence
  Humanised sequence
QVQLVQSGAEVKKPGSSVKVSCEASGYTFTSYGITWVRQAPGQGLEWMGENYPRSGNTYYNEKFKGRVTITADKSTSTAYMELSSLRSEDTAVYYCARSEFISTVVAPYYYALDYWGQGTLVTVSS

SEQ ID NO: 108 - protein, 126 aa, Artificial Sequence
  Humanised sequence
QVQLVQSGAEVKKPGSSVKVSCKASGYTFTSYGITWVRQAPGQGLEWMGENYPRSGNTYYNEKFKGRVTITADKSTNTAYMELSSLRSEDTAVYYCARSEFISTVVAPYYYALDYWGQGTLVTVSS

SEQ ID NO: 109 - protein, 126 aa, Artificial Sequence
  Humanised sequence
QVQLVQSGAEVKKPGSSVKVNCKASGYTFTSYGITWVRQAPGQGLEWMGENYPRSGNTYYNEKFKGRVTITADKSTSTAYMELSSLRSEDTAVYYCARSEFISTVVAPYYYALDYWGQGTLVTVSS

SEQ ID NO: 110 - protein, 126 aa, Artificial Sequence
  Humanised sequence
QVQLVQSGAEVKKPGSSVKVSCKASGYTFTSYGITWVRQAPGQGLEWMGENYPRSGNTYYNEKFRGRVTITADKSTSTAYMELSSLRSEDTAVYYCARSEFISTVVAPYYYALDYWGQGTLVTVSS

SEQ ID NO: 111 - protein, 126 aa, Artificial Sequence
  Humanised sequence
QVQLVQSGAEVKKPGSSVKVSCKASGYTFTSYGITWVRQAPGQGLEWMGENYPRSGNIYYNEKFKGRVTITADKSTSTAYMELSSLRSEDTAVYYCARSEFISTVVAPYYYALDYWGQGTLVTVSS

SEQ ID NO: 112 - protein, 126 aa, Artificial Sequence
  Humanised sequence
QVQLVQSGAEVKKPGSSVKVSCKASGYTFTSYGITWVRQAPGQGLEWMGENYPRSGNTYYNEKFKGRVTITADKSTSTAYMELSGLRSEDTAVYYCARSEFISTVVAPYYYALDYWGQGTLVTVSS

SEQ ID NO: 113 - protein, 126 aa, Artificial Sequence
  Humanised sequence
QVQLVQSGAEVKKPGSSVKVSCKASGYTFASYGITWVRQAPGQGLEWMGENYPRSGNTYYNEKFRGRVTITADKSTSTAYMELSSLRSEDTAVYYCARSEFISTVVAPYYYALDYWGQGTLVTVSS

SEQ ID NO: 114 - protein, 126 aa, Artificial Sequence
  Humanised sequence
QVQLVQSSAEVKKPGSSVKVSCKASGYTFASYGITWVRQAPGQGLEWMGENYPRSGNTYYNEKFKGRVTITADKSTSTAYMELSSLRSEDTAVYYCARSEFTSTVVAPYYYALDYWGQGTLVTVSS

SEQ ID NO: 115 - protein, 126 aa, Artificial Sequence
  Humanised sequence
QVQLVQSGAEVKKPGSSVKVSCKASGYTFASYGITWVRQAPGQGLEWMGENYPRSGNTYYNEKFKGRVTITADKSTGTAYMELSSLRSEDTAVYYCARSEFISTVVAPYYYALDYWGQGTLVTVSS

SEQ ID NO: 116 - protein, 111 aa, Artificial Sequence
  Humanised sequence
DIVMTQSPDSLVVSLGERATINCKASKKVTIFGSTSALHWYQQKPGQPPKLIYNGAKLESGVPDRFSGSGSGTDFTLTISSLQAEDVAVYYCLQNKEVPYTFGGGTKVEIK

SEQ ID NO: 117 - protein, 111 aa, Artificial Sequence
  Humanised sequence
DIVMTQSPDSLAVSLGERATINCKASKKVTIFGSISALHWYQQKPGQPPKLVYNGAKLESGVPDRFSGSGSGADFTLTISSLQAEDVAVYYCLQNKEVPYTFGGGTKVEIK

SEQ ID NO: 118 - protein, 111 aa, Artificial Sequence
  Humanised sequence
DIVMTQSPDSLAVSLGERATINCKASKKVTIFGSISALHWYQQRPGQPPKLIYNGAKLESGVPDRFSGSGSGTDFTLTISSLQAEDVAVYYCLQNKEVPYTFGGGTKVEIK

SEQ ID NO: 119 - protein, 111 aa, Artificial Sequence
  Humanised sequence
DIVMTQSPDSLAVSLGERATINCKASKKVTIFGSISALHWYQQKPGQPPKLIYNGAKLESGVPGRFSGSGSGTDFTLTISSLQAEDVAVYYCLQNKEVPYTFGGGTKVEIK

SEQ ID NO: 120 - protein, 111 aa, Artificial Sequence
  Humanised sequence
DIVMTQSPDSLAVSLGERATINCKASKKVTIFGSISALHWYQQKPGQPPKLIYNSAKLESGVPDRFSGSGSGTDFTLTISSLQAEDVAVYYCLQNKEVPYTFGGGTKVEIK

SEQ ID NO: 121 - protein, 111 aa, Artificial Sequence
  Humanised sequence
DIVMTQSPDSLAVSLGERATINCKASKKVTIYGSTSALHWYQQKPGQPPKLIYNGAKPESGVPDRFSGSGSGTDFTLTISSLQAEDVAVYYCLQNKEVPYTFGGGTKVEIK

SEQ ID NO: 122 - protein, 111 aa, Artificial Sequence
  Humanised sequence
DIVMTQSPDSLAVSLGERATISCKASKKVTIFGSTSALHWYQQKPGQPPKLIYNGAKPESGVPDRFSGSGSGTDFTLTISSLQAEDVAVYYCLQNKEVPYTFGGGTKVEIK

SEQ ID NO: 123 - protein, 111 aa, Artificial Sequence
  Humanised sequence
GIVMTQSPDSLAVSLGERATINCKASKKVTIFGSTSALHWYQQKPGQPPKLIYNGAKLESGVPDRFSGSGSGTDFTLTISSLQAEDVAVYYCLQNKEVPYTFGGGTKVEIK

(19) United States
(12) Patent Application Publication
(10) Pub. No.: US 2011/0206686 A1
(43) Pub. Date: Aug. 25, 2011

(54) IL-23 ANTIBODIES

(76) Inventors: Gary Peter Bembridge, Hertfordshire (GB); Jane Elizabeth Clarkson, Hertfordshire (GB); Jonathan Henry Ellis, Hertfordshire (GB); Paul Andrew Hamblin, Hertfordshire (GB); George Kopsidas, Victoria (AU); Alan Peter Lewis, Hertfordshire (GB); Ruth Mcadam, Hertfordshire (GB)

(21) Appl. No.: 12/681,413

(22) PCT Filed: Oct. 3, 2008
(86) PCT No.: PCT/EP08/63289

§ 371 Date: Apr. 2, 2010

Related U.S. Application Data
(60) Provisional application No. 60/977,841, filed on Oct. 5, 2007.

(51) Int. Cl. A61K 39/395; C07K 16/24; C12N 1/00; C12N 5/10; A61P 29/00; A61P 17/06; A61P 19/02; A61P 25/00; A61P 11/00; A61P 3/10; A61P 11/06; A61P 37/08; A61P 37/06

(52) U.S. Cl. 424/158.1; 530/389.2; 530/387.3; 435/243; 435/335

(57) ABSTRACT

The present invention relates to antigen binding proteins to human IL-23, pharmaceutical formulations containing them and to the use of such antigen binding proteins in the treatment and/or prophylaxis of inflammatory diseases such as Rheumatoid Arthritis (RA).

### FIELD OF THE INVENTION

The present invention relates to antigen binding proteins, particularly antibodies that bind to interleukin 23 (IL-23) and neutralise the activity thereof, polynucleotides encoding such antigen binding proteins, pharmaceutical formulations containing said antigen binding proteins and to the use of such antigen binding proteins in the treatment and/or prophylaxis of diseases associated with inflammation, such as Rheumatoid Arthritis (RA). Other aspects, objects and advantages of the present invention will become apparent from the description below.

### BACKGROUND OF THE INVENTION

Interleukin-23 (IL-23) is a member of the IL-12 heterodimeric cytokine family and contains the p40 chain, which is common to IL12 and IL-23, and a p19 chain which is unique to IL-23. IL-12 is a heterodimer of p40 and its partner p35 which is unique to IL-12.

As with previous studies that demonstrated IL-12p35 requires IL-12p40 for secretion, it was also revealed that secretion of p19 depends on its ability to partner with p40 (Oppmann et al. 715-25). An additional IL-12 family member consisting of a p28 subunit that partners with the Epstein-Barr virus-induced molecules 3 (EBI3) has been designated IL-27 (Pflanz et al. Immunity. 16.6 (2002): 779-90).

The innate ability to distinguish different classes of pathogens (via recognition of conserved molecular patterns shared among large classes of pathogens) provides appropriate information with which to tailor the adaptive response for the selection, activation and expansion of antigen-specific T and B cells. The cytokines IL-12, IL-23 and IL-27 produced by antigen presenting cells (APC) in response to a variety of pathogens are key regulatory molecules that shape these responses.

The seminal work of Mosmann & Coffman in 1986 (Mosmann et al. J. Immunol. 175.1 (2005): 5-14) describing the properties of murine CD4⁺ T helper cell clones that could be subdivided into two subgroups (termed Th1 and Th2) based upon the cytokines they produced provided a basis for the distinct types of immune responses elicited during infection or vaccination. The consequences of elicitation of the appropriate Th1 or Th2 immune response are profound—not only in murine models but also in disease outcome in man. Hence, Th1 CD4+ T cells, characterised by IFNg production are critical for appropriate control of intracellular infections caused by organisms such as Mycobactoerium leprae , Mycobacterium tuberculosis and leshmania donovani in both human disease and in vivo animal models. In contrast, the preferential induction of Th2 CD4⁺ T cells, characterised by production of IL4, IL5 and IL13 cytokines is associated with protection against certain helminth infections as well as IgE associated allergic responses such as asthma and allergic rhinitis. In murine models, mice susceptible to intracellular pathogens (due to predominant Th2 immune responses) could be made resistant by appropriate administration of IL-12 and conversely resistant mice made susceptible by administration of neutralising anti-p40 antibodies. Such studies identified that IL-12 is a pivotal cytokine involved in the differentiation of Th1 cells.

Indeed for many years Th1 CD4⁺ T cells, induced by IL-12, were thought to be responsible for the induction of a wide variety of autoimmune diseases based on the use of neutralising p40 antibodies or p40 knockout mice including experimental autoimmune encephalomyelitis (EAE), collagen-induced arthritis (CIA), inflammatory colitis and autoimmune uveitis. Although such diseases where characterised by high levels of IFNγ (a prototypical Th1 cytokine) the actual role of this cytokine in autoimmune inflammation was less well understood. This can be illustrated by the role of p40 and IFNγ in central nervous system (CNS) inflammation during EAE. Animals that lack IFNγ or IFNγ-mediated signalling (ifn-, ifnr-, and stati-deficient mice) remain susceptible and disease onset is quicker with a more severe pathology (Langrish et al. Immunol. Rev. 202 (2004): 96-105; Langrish et al. J. Exp. Med. 201.2 (2005): 233-40; Mosmann et al. 5-14). Treatment with p40 antibodies inhibited EAE onset. Similar observations have been noted with CIA models. Treatment with p40 neutralising antibodies prevented disease whilst the absence of IFNγ signalling pathway results in increased severity of disease. In addition, IL-12 p35 deficient animals were fully susceptible to EAE which suggested additional roles for p40, that is, additional p40 cytokines to IL-12.

The identification of IL-23 and the realisation that the IL-12 p40 chain is shared by these two cytokines provided an explanation for the observed disparity between the need for p40 and not other Th1 pro-inflammatory cytokines in the propagation of autoimmune responses. This hypothesis has been confirmed in studies using p19 deficient animals. Such animals are completely resistant to EAE and CIA in a manner similar to p40 deficient animals. Furthermore, the finding that stimulation of memory T cells in the presence of IL-23 (but not IL-12) led to the production of IL-17 provided evidence of the unique role of IL-23 in the regulation of effector T cell function. Further studies, including gene expression studies, revealed that IL-23-dependant CD4⁺ T cell populations displayed a distinct profile from IL-12 derived Th1 cells. Subsequent in vivo studies have established the role of IL-23 driven IL-17 producing cells in EAE with as few as 10⁵ CNS antigen-specific IL-17-producing CD4⁺ T cells inducing disease following adoptive transfer into naïve recipients (Langrish et al. 233-40). IL-23 deficient mice (p19^{-/-}) are resistant to CIA and this correlates with a lack of CD4⁺ T cells that make IL-17, a cytokine with a major role in bone catabolism (Murphy et al. J. Exp. Med. 198.12 (2003): 1951-57). The development of spontaneous colitis in IL-10 deficient mice is completely prevented when crossed onto IL-23p19 deficient animals, demonstrating an obligatory role for this cytokine in the induction of colitis (Yen et al. J. Clin. Invest 116.5 (2006): 1310-16). Although recent findings on the role of the IL-23/IL-17 immune axis have explained their role in autoimmune inflammation, it does not explain the exacerbated disease observed in IFNγ signalling deficient mice. Such observations do suggest that IFNγ (or IFNγ-mediated signalling) is part of a regulatory system to counterbalance the effects of IL-23.

Recent studies with human CD4+ T cells have also indicated a role of IL-23 in the differentiation or maintenance of CD4+ IL17 producing T cells (Wilson et al Nature Immunology (2007) δ 950-957), in that IL-23R positive T cells were able to produce quantitatively higher levels of IL17A than IL-23R negative cells. Immunohistochemistry analysis has also demonstrated increased expression of IL-23 p19 by dendritic cells in lesional versus non-lesional skin from patient biopsies with psoriasis.

Additional justification for targeting the IL-23 pathway has emerged from genome-wide association studies that have identified the IL-23 pathway and associated single nucleotide polymorphisms (SNPs) as risk factors for a number of inflammatory diseases. The IL-12/IL-23 pathway has been implicated in psoriasis with the identification of two psoriasis susceptibility genes IL12B and IL-23R (Cargill et al. Am. J. Hum. Genet. 80.2 (2007): 273-90). Similar studies have also identified uncommon coding variants of IL-23R that confer strong protection against Crohn's disease (Duerr et al. Science 314.5804 (2006): 1461-63). Such findings have been confirmed in the British population by the Wellcome Trust case Control Consortium that similarly observed association at many previously identified loci, including SNPs within IL-23R. The rare allele of the R381Q SNP that confers protection against crohns disease in the adult population was negatively associated with inflammatory bowel disease (IBD) in children extending the role of the IL-23 inflammatory pathway into paediatric crohns disease (Dubinsky et al. Inflamm. Bowel. Dis. 13.5 (2007): 511-15).

The identification of susceptibility variants and the growing understanding of the role of the IL-23R pathway in crohns disease, psoriasis and other autoimmune inflammatory disorders should lead to improved therapeutic interventions targeting this pathway. In support of this, a monoclonal antibody against the IL-12, IL-23 shared subunit p40 induced clinical responses and remissions in patients with active crohns disease (Mannon et al. N. Engl. J. Med. 351.20 (2004): 2069-79) and demonstrate therapeutic efficacy in psoriasis (Gottlieb et al. Curr. Med. Res. Opin. 23.5 (2007): 1081-92; Krueger et al. N. Engl. J. Med. 356.6 (2007): 580-92). Although initial studies in psoriatics with anti-p40 mAbs had serious adverse events including myocardial infarctions (Krueger et al. 580-92) there was no evidence of this in a second study (Gottlieb et al. 1081-92). However, it has been postulated that specific-blockade of the IL-23R pathway may be effective in blocking organ-specific inflammation without fully compromising protective responses (McKenzie, Trends Immunol. 27.1 (2006): 17-23).

There are several anti-IL-23 specific mAbs described in the art. These include mAbs that bind specific portions of the p19 subunit of IL-23 (WO2007/024846, WO 2007/005955) or mAbs that bind IL-23p40 specific sequences and not bind the p40 subunit of IL12 (US 2005/0137385 A1). In addition, mAbs that bind p40 (common to IL12 and IL-23) and neutralise both IL12 and IL-23 have shown clinical efficacy in psoriasis (Gottlieb et al. Current Med. Res. & Op 23 (2007): 1081-1092) and crohn's disease (Mannon et al. N. Eng. J. Med 351 (2004): 2069-2079).

Despite the art providing anti IL-23 antibodies, it remains a highly desirable goal to isolate and develop therapeutically useful antigen binding proteins, such as monoclonal antibodies that bind and inhibit the activity of human IL-23.

Antigen binding proteins for the treatment of the above mentioned disease/disorders are provided by the present invention and described in detail below.

### BRIEF SUMMARY OF THE INVENTION

The invention provides antigen binding proteins which bind to IL-23, for example antibodies that bind IL-23. Certain embodiments of the present invention include monoclonal antibodies (mAbs) related to, or derived from, a murine mAb 8C9 2H6. The 8C9 2H6 heavy chain variable region amino acid sequence is provided as SEQ ID NO.8. The 8C9 2H6 light chain variable region amino acid sequence is provided as SEQ ID NO.10.

The heavy chain variable regions (VH) of the present invention comprise the following CDRs (as defined by Kabat):

TABLE 1
The CDRs of the heavy chain variable regions
of the present invention may comprise the
following CDRs

| CDR | According to Kabat |
|---|---|
| H1 | SYGIT (SEQ ID NO: 1) |
| H2 | ENYPRSGNTYYNEKFKG |
| | ( |
| H3 | CEFISTVVAPYYYALDY |
| | ( |
| H3 alternative | SEFISTVVAPYYYALDY |
| | ( |

or the alternative CDRs set out in SEQ ID NO:72, SEQ ID NO:73, SEQ ID NO: 74, SEQ ID NO: 95, SEQ ID NO: 98, SEQ ID NO: 99 and SEQ ID NO:100.

The light chain variable regions of the present invention comprise the following CDRs (as defined by Kabat):

| CDR | According to Kabat |
|---|---|
| L1 | KASKKVTIFGSISALH |
| | ( |
| L2 | NGAKLES |
| | ( |
| L3 | LQNKEVPYT |
| | ( |

or the alternative CDRs set out in SEQ ID NO:75, SEQ ID NO:76, SEQ ID NO:77, SEQ ID NO:78, SEQ ID NO:79, SEQ ID NO:80, SEQ ID NO:101 and SEQ ID NO:102.

In one embodiment the antigen binding proteins of the present invention comprise a heavy chain variable region containing a CDRH3 selected from the list consisting of SEQ ID NO: 3, SEQ ID NO: 4, SEQ ID NO: 73, SEQ ID NO: 74, SEQ ID NO:95 and SEQ ID NO: 100, paired with a light chain variable region to form an antigen binding Fv unit which binds to human IL-23 and neutralises the activity of human IL-23. In one aspect of this embodiment the CDRH1 as set out in SEQ ID NO: 1, and CDRH2 selected from the list consisting of SEQ ID NO:2, SEQ ID NO:72, SEQ ID NO:98 and SEQ ID NO: 99, are also present in the heavy chain variable region. In another aspect the antigen binding Fv unit binds to human IL-23 with high affinity as measured by Biacore of 10 nM or less, and more particularly 2 nM or less, for example between about 0.8 nM and 2 nM, 1 nM or less, or 100 pM or less. In one such embodiment, this is measured by Biacore with the antigen binding Fv unit being captured on the biosensor chip, for example as set out in Example 5.

The heavy chain variable regions of the present invention may be formatted together with light chain variable regions to allow binding to human IL-23, in the conventional immunoglobulin manner (for example, human IgG, IgA, IgM etc.) or in any other “antibody-like” format that binds to human IL-23 (for example, single chain Fv, diabodies, Tandabs™ etc (for a summary of alternative “antibody” formats see Holliger and Hudson, Nature Biotechnology, 2005, Vol 23, No. 9, 1126-1136)).

The antigen binding proteins of the present invention include the murine antibody having the variable regions as described in SEQ ID NO:8 and SEQ ID NO:10 or non-murine equivalents thereof, such as rat, human, chimeric or humanised variants thereof.

The term “binds to human IL-23” as used throughout the present specification in relation to antigen binding proteins thereof of the invention means that the antigen binding protein binds human IL-23 (hereinafter referred to as hIL-23) with no or insignificant binding to other human proteins such as IL-12. In particular the antigen binding proteins of the present invention bind to human IL-23 in that they can be seen to bind to human IL-23 in a Biacore assay (for example the Biacore assay described in example 5), whereas they do not bind or do not bind significantly to human IL-12 in an equivalent Biacore assay. The term however does not exclude the fact that certain antigen binding proteins of the invention may also be cross-reactive with IL-23 from other species, for example cynomolgus IL-23.

The term “antigen binding protein” as used herein refers to antibodies, antibody fragments and other protein constructs which are capable of binding to and neutralising human IL-23.

In another aspect of the invention there is provided an antigen binding protein, for example an antibody which binds human IL-23 and comprises a CDRH3 which is a variant of the sequence set forth in SEQ ID NO: 3 in which one or two residues within said CDRH3 of said variant differs from the residue in the corresponding position in SEQ ID NO: 3, for example the first residue of SEQ ID NO: 3 (cysteine) is substituted for a different amino acid, for example the CDRs having the sequence of SEQ ID NO:4 or SEQ ID NO:73 or SEQ ID NO:74, and/or for example the eighth residue of SEQ ID NO: 3 (valine) is substituted for a different amino acid, for example as set out in SEQ ID NO: 95, so in one aspect variants of CDRH3 have one residue that differs from CDRH3 of SEQ ID NO: 3, for example at position 1 or position 8, for example the amino acid residue at position 1 of CDRH3 is selected from cysteine, serine, alanine and valine, and for example the amino acid residue at position 8 of CDRH3 is selected from valine and methionine. In another aspect variants of CDRH3 include substitutions at both positions 1 and 8, for example as set out in SEQ ID NO: 95.

In a further aspect of the invention CDRH3 comprises a variant of the sequence set forth in SEQ ID NO: 3 in which one, two or three residues within said CDRH3 of said variant differs from the residue in the corresponding position in SEQ ID NO: 3, wherein the fourth residue of SEQ ID NO: 3 (isloleucine) is substituted for a different amino acid, for example the CDRs having the sequence of SEQ ID NO:100, for example the amino acid residue at position four of CDRH3 may be threonine. In addition, such variants may also comprise one or both of the substitutions described above at positions one and eight.

In one aspect the antigen binding proteins of the present invention, for example antibodies, comprise CDRH1 as set out in SEQ ID NO: 1, CDRH2 as set out in SEQ ID NO:2, SEQ ID NO: 72, SEQ ID NO:98 or SEQ ID NO: 99, CDRH3 as set out in SEQ ID NO: 3, SEQ ID NO:4, SEQ ID NO:73, SEQ ID NO: 74, SEQ ID NO: 95 or SEQ ID NO: 100, CDRL1 as set out in SEQ ID NO: 5, SEQ ID NO: 75, or SEQ ID NO: 101, CDRL2 as set out in SEQ ID NO: 6, SEQ ID NO: 76, SEQ ID NO: 77, SEQ ID NO: 78, SEQ ID NO: 79, SEQ ID NO: 80 or SEQ ID NO: 102, and CDRL3 as set out in SEQ ID NO: 7. In one such embodiment the antigen binding protein, for example an antibody, comprises the following CDRs:

CDRH1: SEQ. I.D. NO: 1

CDRH2: SEQ. I.D. NO: 2

CDRH3: SEQ. I.D. NO: 4

CDRL1: SEQ. I.D. NO: 5

CDRL2: SEQ. I.D. NO: 6

CDRL3: SEQ. I.D. NO: 7

In another aspect of the invention there is provided an antigen binding protein, for example an antibody which binds human IL-23 and comprises the CDRs as set out in:

CDRH1: SEQ ID NO: 1

CDRH2: SEQ ID NO: 2

CDRH3: SEQ ID NO: 4

CDRL1: SEQ ID NO: 5

CDRL2: SEQ ID NO: 6 and

CDRL3: SEQ ID NO: 7

or variants of any one or more of these CDRS in which one or two residues, or in which up to three residues within each CDR sequence of said variant differs from the residue in the corresponding position in the SEQ ID NO: listed above, for example those CDRs set out in SEQ ID NOs: SEQ ID NO: 3, SEQ ID NO: 72, SEQ ID NO: 98, SEQ ID NO: 99, SEQ ID NO:73, SEQ ID NO:74, SEQ ID NO: 95, SEQ ID NO: 100, SEQ ID NO: 75, SEQ ID NO: 101, SEQ ID NO:76, SEQ ID NO: 77, SEQ ID NO:78, SEQ ID NO:79, SEQ ID NO:80 and SEQ ID NO:102.

Throughout this specification, amino acid residues in antibody sequences are numbered according to the Kabat scheme. Similarly, the terms “CDR”, “CDRL1”, “CDRL2”, “CDRL3”, “CDRH1”, “CDRH2”, “CDRH3” follow the Kabat numbering system as set forth in Kabat et al; “Sequences of proteins of Immunological Interest” NIH, 1987.

In another aspect of the invention there is provided an antigen binding protein, such as a humanised antibody or antigen binding fragment thereof, comprising a VH domain having the sequence set forth in SEQ ID NO: 16, SEQ ID NO: 48, SEQ ID NO: 50, SEQ ID NO: 52, SEQ ID NO: 54, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114 or SEQ ID NO: 115; and a VL domain having the sequence set forth in SEQ ID NO:18, SEQ ID NO: 20, SEQ ID NO:22, SEQ ID NO: 24, SEQ ID NO:56, SEQ ID NO:58, SEQ ID NO:96, SEQ ID NO: 97, SEQ ID NO:116, SEQ ID NO: 117, SEQ ID NO:118, SEQ ID NO:119, SEQ ID NO:120, SEQ ID NO:121, SEQ ID NO:122 or SEQ ID NO: 123. It is intended that this list of VH and VL sequences specifically discloses all possible combinations of any individual VH and any individual VL sequences.

The heavy chain variable regions of the present invention may comprise CDRH1 as set out in SEQ ID NO: 1, CDRH2 as set out in SEQ ID NO: 2, SEQ ID NO: 72, SEQ ID NO: 98, or SEQ ID NO: 99, and CDRH3 as set out in SEQ ID NO: 3, SEQ ID NO:4, SEQ ID NO:73, SEQ ID NO: 74, SEQ ID NO:95, or SEQ ID NO: 100. For example, the heavy chain variable region of the present invention may comprise CDRH1 as set out in SEQ ID NO: 1, CDRH2 as set out in SEQ ID NO:2, and CDRH3 as set out in SEQ ID NO: 3. Alternatively it may comprise CDRH1 as set out in SEQ ID NO: 1, CDRH2 as set out in SEQ ID NO:2, and CDRH3 as set out in SEQ ID NO: 4, or it may comprise CDRH1 as set out in SEQ ID NO: 1, CDRH2 as set out in SEQ ID NO:2, and CDRH3 as set out in SEQ ID NO: 73, or it may comprise CDRH1 as set out in SEQ ID NO: 1, CDRH2 as set out in SEQ ID NO:2, and CDRH3 as set out in SEQ ID NO: 74, or it may comprise CDRH1 as set out in SEQ ID NO: 1, CDRH2 as set out in SEQ ID NO:72, and CDRH3 as set out in SEQ ID NO: 3, or it may comprise CDRH1 as set out in SEQ ID NO: 1, CDRH2 as set out in SEQ ID NO:72, and CDRH3 as set out in SEQ ID NO: 4, or it may comprise CDRH1 as set out in SEQ ID NO: 1, CDRH2 as set out in SEQ ID NO:72, and CDRH3 as set out in SEQ ID NO: 73, or it may comprise CDRH1 as set out in SEQ ID NO: 1, CDRH2 as set out in SEQ ID NO:72, and CDRH3 as set out in SEQ ID NO: 74, or it may comprise CDRH1 as set out in SEQ ID NO: 1, CDRH2 as set out in SEQ ID NO:2, and CDRH3 as set out in SEQ ID NO: 95, or it may comprise CDRH1 as set out in SEQ ID NO: 1, CDRH2 as set out in SEQ ID NO:72, and CDRH3 as set out in SEQ ID NO: 95, or it may comprise CDRH1 as set out in SEQ ID NO: 1, CDRH2 as set out in SEQ ID NO:98, and CDRH3 as set out in SEQ ID NO: 3, or it may comprise CDRH1 as set out in SEQ ID NO: 1, CDRH2 as set out in SEQ ID NO:98, and CDRH3 as set out in SEQ ID NO: 4, or it may comprise CDRH1 as set out in SEQ ID NO: 1, CDRH2 as set out in SEQ ID NO:98, and CDRH3 as set out in SEQ ID NO: 73, or it may comprise CDRH1 as set out in SEQ ID NO: 1, CDRH2 as set out in SEQ ID NO:98, and CDRH3 as set out in SEQ ID NO:74, or it may comprise CDRH1 as set out in SEQ ID NO: 1, CDRH2 as set out in SEQ ID NO:98, and CDRH3 as set out in SEQ ID NO: 95, or it may comprise CDRH1 as set out in SEQ ID NO: 1, CDRH2 as set out in SEQ ID NO:98, and CDRH3 as set out in SEQ ID NO: 100, or it may comprise CDRH1 as set out in SEQ ID NO: 1, CDRH2 as set out in SEQ ID NO:99 and CDRH3 as set out in SEQ ID NO: 3, or it may comprise CDRH1 as set out in SEQ ID NO: 1, CDRH2 as set out in SEQ ID NO:99 and CDRH3 as set out in SEQ ID NO: 4, or it may comprise CDRH1 as set out in SEQ ID NO: 1, CDRH2 as set out in SEQ ID NO:99 and CDRH3 as set out in SEQ ID NO: 73, or it may comprise CDRH1 as set out in SEQ ID NO: 1, CDRH2 as set out in SEQ ID NO:99 and CDRH3 as set out in SEQ ID NO: 74, or it may comprise CDRH1 as set out in SEQ ID NO: 1, CDRH2 as set out in SEQ ID NO:99 and CDRH3 as set out in SEQ ID NO: 95, or it may comprise CDRH1 as set out in SEQ ID NO: 1, CDRH2 as set out in SEQ ID NO:99 and CDRH3 as set out in SEQ ID NO: 100, or it may comprise CDRH1 as set out in SEQ ID NO: 1, CDRH2 as set out in SEQ ID NO:2 and CDRH3 as set out in SEQ ID NO: 100, or it may comprise CDRH1 as set out in SEQ ID NO: 1, CDRH2 as set out in SEQ ID NO:72 and CDRH3 as set out in SEQ ID NO: 100.

The light chain variable regions of the present invention may comprise CDRL1 as set out in SEQ ID NO: 5, SEQ ID NO: 75 or SEQ ID NO: 101, CDRL2 as set out in SEQ ID NO: 6, SEQ ID NO: 76, SEQ ID NO: 77, SEQ ID NO: 78, SEQ ID NO: 79, SEQ ID NO: 80 or SEQ ID NO: 102, and CDRL3 as set out in SEQ ID NO: 7. For example, the light chain variable region of the present invention may comprise CDRL1 as set out in SEQ ID NO: 5, CDRL2 as set out in SEQ ID NO: 6, and CDRL3 as set out in SEQ ID NO: 7, or it may comprise CDRL1 as set out in SEQ ID NO: 5, CDRL2 as set out in SEQ ID NO: 76, and CDRL3 as set out in SEQ ID NO: 7, or it may comprise CDRL1 as set out in SEQ ID NO: 5, CDRL2 as set out in SEQ ID NO: 77, and CDRL3 as set out in SEQ ID NO: 7, or it may comprise CDRL1 as set out in SEQ ID NO: 5, CDRL2 as set out in SEQ ID NO: 78, and CDRL3 as set out in SEQ ID NO: 7, or it may comprise CDRL1 as set out in SEQ ID NO: 5, CDRL2 as set out in SEQ ID NO: 79, and CDRL3 as set out in SEQ ID NO: 7, or it may comprise CDRL1 as set out in SEQ ID NO: 5, CDRL2 as set out in SEQ ID NO: 80, and CDRL3 as set out in SEQ ID NO: 7, or it may comprise CDRL1 as set out in SEQ ID NO: 75, CDRL2 as set out in SEQ ID NO: 6, and CDRL3 as set out in SEQ ID NO: 7, or it may comprise CDRL1 as set out in SEQ ID NO: 75, CDRL2 as set out in SEQ ID NO: 76, and CDRL3 as set out in SEQ ID NO: 7, or it may comprise CDRL1 as set out in SEQ ID NO: 75, CDRL2 as set out in SEQ ID NO: 77, and CDRL3 as set out in SEQ ID NO: 7, or it may comprise CDRL1 as set out in SEQ ID NO: 75, CDRL2 as set out in SEQ ID NO: 78, and CDRL3 as set out in SEQ ID NO: 7, or it may comprise CDRL1 as set out in SEQ ID NO: 75, CDRL2 as set out in SEQ ID NO: 79, and CDRL3 as set out in SEQ ID NO: 7, or it may comprise CDRL1 as set out in SEQ ID NO: 75, CDRL2 as set out in SEQ ID NO: 80, and CDRL3 as set out in SEQ ID NO: 7, or it may comprise CDRL1 as set out in SEQ ID NO: 101, CDRL2 as set out in SEQ ID NO: 6, and CDRL3 as set out in SEQ ID NO: 7, or it may comprise CDRL1 as set out in SEQ ID NO: 101, CDRL2 as set out in SEQ ID NO: 76, and CDRL3 as set out in SEQ ID NO: 7, or it may comprise CDRL1 as set out in SEQ ID NO: 101, CDRL2 as set out in SEQ ID NO: 77, and CDRL3 as set out in SEQ ID NO: 7, or it may comprise CDRL1 as set out in SEQ ID NO: 101, CDRL2 as set out in SEQ ID NO: 78, and CDRL3 as set out in SEQ ID NO: 7, or it may comprise CDRL1 as set out in SEQ ID NO: 101, CDRL2 as set out in SEQ ID NO: 79, and CDRL3 as set out in SEQ ID NO: 7, or it may comprise CDRL1 as set out in SEQ ID NO: 101, CDRL2 as set out in SEQ ID NO: 80, and CDRL3 as set out in SEQ ID NO: 7, or it may comprise CDRL1 as set out in SEQ ID NO: 101, CDRL2 as set out in SEQ ID NO: 102, and CDRL3 as set out in SEQ ID NO: 7, or it may comprise CDRL1 as set out in SEQ ID NO: 5, CDRL2 as set out in SEQ ID NO: 102, and CDRL3 as set out in SEQ ID NO: 7, or it may comprise CDRL1 as set out in SEQ ID NO: 75, CDRL2 as set out in SEQ ID NO: 102, and CDRL3 as set out in SEQ ID NO: 7.

Any of these heavy chain variable regions may be combined with any of the light chain variable regions, for example the antigen binding protein of the present invention may comprise a heavy chain variable region comprising CDRH1 as set out in SEQ ID NO:1, CDRH2 as set out in SEQ ID NO:2, SEQ ID NO:72, SEQ ID NO:98 or SEQ ID NO:99, and CDRH3 as set out in SEQ ID NO:4, SEQ ID NO:73 or SEQ ID NO:74, combined with a light chain variable region comprising CDRL1 as set out in SEQ ID NO: 75 or SEQ ID NO:101, a CDRL2 as set out in SEQ ID NO:6 or SEQ ID NO:76 and CDRL3 as set out in SEQ ID NO:7.

Any of the heavy chain variable regions of the invention can be combined with a suitable human constant region, such as that set out in SEQ ID NO:92, to provide a full length heavy chain. Any of the light chain variable regions of the invention can be combined with a suitable human constant region, such as that set out in SEQ ID NO:91, to provide a full length light chain.

The heavy chain variable region constructs of the present invention may be paired with a light chain to form an human IL-23 binding unit (Fv) in any format, including a conventional IgG antibody format. Examples of full length (FL) heavy chain sequences comprising the VH constructs of the present invention include SEQ ID NO: 26, 60, 62, 64, and 66.

The light chain variable region sequence that forms an Fv with the heavy chain variable region sequences of the present invention may be any sequence that allows the Fv to bind to Human IL-23. Examples of full length (FL) light chain sequences comprising the VH constructs of the present invention include SEQ ID NO:28, 30, 32, 34, 68, 70, 93 and 94.

In particular embodiments the antigen binding proteins of the present invention comprise the following variable region pairs:

A3M0 (SEQ ID NO:16+SEQ ID NO: 18)

A3M1 (SEQ ID NO:16+SEQ ID NO: 20)

A3N1 (SEQ ID NO:16+SEQ ID NO: 22)

A3N2 (SEQ ID NO:16+SEQ ID NO: 24)

A7M3 (SEQ ID NO: 52+SEQ ID NO: 56)

A10M3 (SEQ ID NO: 54+SEQ ID NO: 56)

A3M4 (SEQ ID NO: 16+SEQ ID NO: 58)

A5M0 (SEQ ID NO: 48+SEQ ID NO: 18)

A6M0 (SEQ ID NO: 50+SEQ ID NO: 18)

A8M3 (SEQ ID NO: 81+SEQ ID NO: 56)

A9M3 (SEQ ID NO: 82+SEQ ID NO: 56)

A10.5M3 (SEQ ID NO: 85+SEQ ID NO: 56)

A11M3 (SEQ ID NO: 83+SEQ ID NO: 56)

A12M3 (SEQ ID NO: 84+SEQ ID NO: 56)

A11.5M3 (SEQ ID NO: 86+SEQ ID NO: 56)

A12.5M3 (SEQ ID NO: 87+SEQ ID NO: 56)

A8M4 (SEQ ID NO: 81+SEQ ID NO: 58)

A9M4 (SEQ ID NO: 82+SEQ ID NO: 58)

A10.5M4 (SEQ ID NO: 85+SEQ ID NO: 58)

A11M4 (SEQ ID NO: 83+SEQ ID NO: 58)

A11.5M4 (SEQ ID NO: 86+SEQ ID NO: 58)

A12M4 (SEQ ID NO: 84+SEQ ID NO: 58)

A12.5M4 (SEQ ID NO: 87+SEQ ID NO: 58)

A13M4 (SEQ ID NO: 88+SEQ ID NO: 58)

A14M4 (SEQ ID NO: 89+SEQ ID NO: 58)

A15M4 (SEQ ID NO: 90+SEQ ID NO: 58)

A3M12 (SEQ ID NO: 16+SEQ ID NO:121)

A3M13 (SEQ ID NO:26+SEQ ID NO: 88)

A23M4 (SEQ ID NO:110+SEQ ID NO:58)

A10.5M14 (SEQ ID NO: 85+SEQ ID NO:123)

A24M4 (SEQ ID NO:111+SEQ ID NO:58)

In another embodiment the antigen binding proteins, for example, the antibodies of the present invention comprise the following full length sequences:

A3M0 (SEQ ID NO: 26+SEQ ID NO:28)

A3M1 (SEQ ID NO: 26+SEQ ID NO:30)

A3N1 (SEQ ID NO: 26+SEQ ID NO:32)

A3N2 (SEQ ID NO: 26+SEQ ID NO:34)

A5M0 (SEQ ID NO: 60+SEQ ID NO:28)

A6M0 (SEQ ID NO: 62+SEQ ID NO:28)

A7M3 (SEQ ID NO: 64+SEQ ID NO:68)

A3M4 (SEQ ID NO: 26+SEQ ID NO:70)

A3M5 (SEQ ID NO: 26+SEQ ID NO:93)

A3M6 (SEQ ID NO: 26+SEQ ID NO:94)

A5M4 (SEQ ID NO: 60+SEQ ID NO:70)

A6M4 (SEQ ID NO: 62+SEQ ID NO:70)

A7M4 (SEQ ID NO: 64+SEQ ID NO:70)

A10M4 (SEQ ID NO: 66+SEQ ID NO:70)

A10M3 (SEQ ID NO: 66+SEQ ID NO:68)

In one embodiment the antigen binding protein of the present invention may be a multi-specific antibody which comprises one or more CDRs of the present invention, which is capable of binding to IL-23 and which is also capable of binding to one or more TH17 type cytokines, for example. IL-17, IL-22, or IL-21. In one such embodiment, a multi-specific antibody is provided which comprises a CDRH3, or an antigen binding protein as defined herein, and which comprises a further antigen binding site which is capable of binding to IL-17, or IL-22, or IL-21. One example of an antigen binding protein of the present invention is an antibody specific for IL-23 comprising a CDRH3 as defined herein, linked to one or more epitope-binding domains which have specificity for one or more TH17 type cytokines, for example. IL-17, IL-22, or IL-21.

As used herein the term “domain” refers to a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

As used herein the term “immunoglobulin single variable domain” refers to an antibody variable domain (V_{H}, V_{HH}, V_{L}) that specifically binds an antigen or epitope independently of a different V region or domain. An immunoglobulin single variable domain can be present in a format (e.g., homo- or hetero-multimer) with other, different variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” which is capable of binding to an antigen as the term is used herein. An immunoglobulin single variable domain may be a human antibody variable domain, but also includes single antibody variable domains from other species such as rodent (for example, as disclosed in WO 00/29004, nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanised according to standard techniques available in the art, and such domains are still considered to be “domain antibodies” according to the invention. As used herein “V_{H} includes camelid V_{HH} domains.

The term “Epitope-binding domain” refers to a domain that specifically binds an antigen or epitope independently of a different V region or domain, this may be a domain antibody or may be a domain which is a derivative of a scaffold selected from the group consisting of CTLA-4, lipocalin, SpA, an Affibody, an avimer, GroEI, transferrin, GroES and fibronectin/adnectin, which has been subjected to protein engineering in order to obtain binding to a ligand other than the natural ligand.

As used herein, the term “antigen binding site” refers to a site on an antigen binding protein which is capable of specifically binding to antigen, this may be a single domain, for example an epitope-binding domain, or single-chain Fv (ScFv) domains or it may be paired VH/VL domains as can be found on a standard antibody.

A further aspect of the invention provides a pharmaceutical composition comprising an antigen binding protein of the present invention together with a pharmaceutically acceptable diluent or carrier.

In a further aspect, the present invention provides a method of treatment or prophylaxis of diseases or disorders associated with an immune system mediated inflammation such as psoriasis, inflammatory bowel disease, ulcerative colitis, crohns disease, rheumatoid arthritis, juvenile rheumatoid arthritis, systemic lupus erythematosus, neurodegenerative diseases, for example multiple sclerosis, neutrophil driven diseases, for example COPD, Wegeners vasculitis, cystic fibrosis, Sjogrens syndrome, chronic transplant rejection, type 1 diabetes graft versus host disease, asthma, allergic diseases atoptic dermatitis, eczematous dermatitis, allergic rhinitis, autoimmune diseases other including thyroiditis, spondyloarthropathy, ankylosing spondylitis, uveitis, polychonritis or scleroderma in a human which comprises administering to said human in need thereof an effective amount of an antigen binding protein of the invention. In one embodiment the disorder is rheumatoid arthritis.

In another aspect, the invention provides the use of an antigen binding protein of the invention in the preparation of a medicament for treatment or prophylaxis of immune system mediated inflammation such as psoriasis, inflammatory bowel disease, ulcerative colitis, crohns disease, rheumatoid arthritis, juvenile rheumatoid arthritis, systemic lupus erythematosus, neurodegenerative diseases, for example multiple sclerosis, neutrophil driven diseases, for example COPD, Wegeners vasculitis, cystic fibrosis, Sjogrens syndrome, chronic transplant rejection, type 1 diabetes graft versus host disease, asthma, allergic diseases atoptic dermatitis, eczematous dermatitis, allergic rhinitis, autoimmune diseases other including thyroiditis, spondyloarthropathy, ankylosing spondylitis, uveitis, polychonritis or scleroderma. In one embodiment the disorder is rheumatoid arthritis.

Other aspects and advantages of the present invention are described further in the detailed description and the preferred embodiments thereof.

In one embodiment, the invention provides antigen binding proteins which compete with an antibody comprising CDRH3 (SEQ ID NO:3, SEQ ID NO:4, SEQ ID NO:73, SEQ ID NO: 74, SEQ ID NO: 95 or SEQ ID NO: 100), for example, the antigen binding protein of the invention competes with an antibody comprising:

### CDRH1: SEQ. I.D. NO: 1

### CDRH2: SEQ. I.D. NO: 2

### CDRH3: SEQ. I.D. NO: 4

### CDRL1: SEQ. I.D. NO: 5

### CDRL2: SEQ. I.D. NO: 6 and

CDRL3: SEQ. I.D. NO: 7,

for binding and neutralising of hIL-23, for example as determined by the inhinition of IL-23 binding to IL-23R ELISA (for example as set out in Example 6), or the inhibition of IL-17 or IL-22 production by splenocytes (for example the bioassay set out in Example 7). In one embodiment the antibody that competes is one which competes with A3M0 (SEQ ID NO: 26, SEQ ID NO: 28).

In another embodiment, the antigen binding protein of the present invention is one which binds to the same epitope as an antibody comprising CDRH3 (SEQ ID NO: 3, SEQ ID NO: 4, SEQ ID NO: 73, SEQ ID NO: 74, SEQ ID NO: 95, or SEQ ID NO: 100), for example the antibody comprising:

### CDRH1: SEQ. I.D. NO: 1

### CDRH2: SEQ. I.D. NO: 2

### CDRH3: SEQ. I.D. NO: 4

### CDRL1: SEQ. I.D. NO: 5

### CDRL2: SEQ. I.D. NO: 6 and

### CDRL3: SEQ. I.D. NO: 7,

In one embodiment the antigen binding protein that competes is one which binds to the same epitope as A3M0 (SEQ ID NO: 26, SEQ ID NO: 28). The epitope can be determined by methods known to one skilled in the art, for example by peptide mapping using a peptide library corresponding the sequence of human p19 (SEQ ID NO:37) each peptide containing 14 amino acid residues, the sequences of each peptide overlapping peptides. Conformational and or Discontinuous epitopes may be identified by known methods for example CLIPS™ (Pepscan Systems).

### DETAILED DESCRIPTION OF THE INVENTION

The antigen binding proteins of the invention may comprise heavy chain variable regions and light chain variable regions of the invention which may be formatted into the structure of a natural antibody or functional fragment or equivalent thereof. An antigen binding protein of the invention may therefore comprise the VH regions of the invention formatted into a full length antibody, a (Fab′)₂ fragment, a Fab fragment, or equivalent thereof (such as scFV, bi- tri- or tetra-bodies, Tandabs, etc.), when paired with an appropriate light chain. The antibody may be an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. Furthermore, the antigen binding protein may comprise modifications of all classes e.g. IgG dimers, Fc mutants that no longer bind Fc receptors or mediate C1q binding. The antigen binding protein may also be a chimeric antibody of the type described in WO86/01533 which comprises an antigen binding region and a non-immunoglobulin region.

The constant region is selected according to any functionality required. An IgG1 may demonstrate lytic ability through binding to complement and/or will mediate ADCC (antibody dependent cell cytotoxicity). An IgG4 will be preferred if a non-cytotoxic blocking antibody is required. However, IgG4 antibodies can demonstrate instability in production and therefore it may be more preferable to modify the generally more stable IgG1. Suggested modifications are described in EP0307434, for example mutations at positions 235 and 237. The invention therefore provides a lytic or a non-lytic form of an antigen binding protein, for example an antibody according to the invention.

In certain forms the antibody of the invention is a full length (e.g. H2L2 tetramer) lytic or non-lytic IgG1 antibody having any of the heavy chain variable regions described herein.

In a further aspect, the invention provides polynucleotides encoding the light and heavy chain variable regions as described herein.

A receptor for the heterodimeric cytokine IL-23 is composed of IL-12Rbeta1 and a novel cytokine receptor subunit, IL-23R. Parham, C. et al J. Immunol. 168 (11), 5699-5708 (2002) (SEQ ID NO:47).

The term “neutralises” and grammatical variations thereof as used throughout the present specification in relation to antigen binding proteins of the invention means that a biological activity of IL-23 is reduced, either totally or partially, in the presence of the antigen binding proteins of the present invention in comparison to the activity of IL-23 in the absence of such antigen binding proteins. Neutralisation may be due to but not limited to one or more of blocking ligand binding, preventing the ligand activating the receptor, down regulating the IL-23 receptor or affecting effector functionality. Levels of neutralisation can be measured in several ways, for example by use of the assays as set out in the examples below, for example in an assay which measures inhibition of IL-23 binding to IL-23 receptor which may be carried out for example as described in Example 6. The neutralisation of IL-23 in this assay is measured by assessing the decreased binding between the IL-23 and its receptor in the presence of neutralising antigen binding protein.

Levels of neutralisation can also be measured, for example in an IL-17 production assay which may be carried out for example as described in Example 7. The neutralisation of IL-23 in this assay is measured by assessing the inhibition of production of IL-17 in the presence of neutralising antigen binding protein.

Other methods of assessing neutralisation, for example, by assessing the decreased binding between the IL-23 and its receptor in the presence of neutralising antigen binding protein are known in the art, and include, for example, Biacore assays.

In an alternative aspect of the present invention there is provided antigen binding proteins which have at least substantially equivalent neutralising activity to the antibodies exemplified herein, for example antigen binding proteins which retain the neutralising activity of A3M1, A3N1, A3N2 or A3M0 in the IL-23/IL-23 receptor neutralisation assay or IL-17/IL-22 production assay, or inhibition of pSTAT3 signalling assay as set out in Examples 6, 7, and 11 respectively.

The terms Fv, Fc, Fd, Fab, or F(ab)₂ are used with their standard meanings (see, e.g., Harlow et al., Antibodies A Laboratory Manual, Cold Spring Harbor Laboratory, (1988)).

A “chimeric antibody” refers to a type of engineered antibody which contains a naturally-occurring variable region (light chain and heavy chains) derived from a donor antibody in association with light and heavy chain constant regions derived from an acceptor antibody.

A “humanised antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., Queen et al., Proc. Natl. Acad Sci USA, 86:10029-10032 (1989), Hodgson et al., Bio/Technology, 9:421 (1991)). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanised antibodies—see for example EP-A-0239400 and EP-A-054951

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p877-883.

The antigen binding proteins, for example antibodies of the present invention may be produced by transfection of a host cell with an expression vector comprising the coding sequence for the antigen binding protein of the invention. An expression vector or recombinant plasmid is produced by placing these coding sequences for the antigen binding protein in operative association with conventional regulatory control sequences capable of controlling the replication and expression in, and/or secretion from, a host cell. Regulatory sequences include promoter sequences, e.g., CMV promoter, and signal sequences which can be derived from other known antibodies. Similarly, a second expression vector can be produced having a DNA sequence which encodes a complementary antigen binding protein light or heavy chain. In certain embodiments this second expression vector is identical to the first except insofar as the coding sequences and selectable markers are concerned, so to ensure as far as possible that each polypeptide chain is functionally expressed. Alternatively, the heavy and light chain coding sequences for the antigen binding protein may reside on a single vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antigen binding protein of the invention. The antigen binding protein which includes the association of both the recombinant heavy chain and/or light chain is screened from culture by appropriate assay, such as ELISA or RIA. Similar conventional techniques may be employed to construct other antigen binding proteins.

Suitable vectors for the cloning and subcloning steps employed in the methods and construction of the compositions of this invention may be selected by one of skill in the art. For example, the conventional pUC series of cloning vectors may be used. One vector, pUC19, is commercially available from supply houses, such as Amersham (Buckinghamshire, United Kingdom) or Pharmacia (Uppsala, Sweden). Additionally, any vector which is capable of replicating readily, has an abundance of cloning sites and selectable genes (e.g., antibiotic resistance), and is easily manipulated may be used for cloning. Thus, the selection of the cloning vector is not a limiting factor in this invention.

The expression vectors may also be characterized by genes suitable for amplifying expression of the heterologous DNA sequences, e.g., the mammalian dihydrofolate reductase gene (DHFR). Other preferable vector sequences include a poly A signal sequence, such as from bovine growth hormone (BGH) and the betaglobin promoter sequence (betaglopro). The expression vectors useful herein may be synthesized by techniques well known to those skilled in this art.

The components of such vectors, e.g. replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the antigen binding proteins of the present invention. Host cells useful for the cloning and other manipulations of these cloning vectors are also conventional. However, cells from various strains of E. coli may be used for replication of the cloning vectors and other steps in the construction of antigen binding proteins of this invention.

Suitable host cells or cell lines for the expression of the antigen binding proteins of the invention include mammalian cells such as NSO, Sp2/0, CHO (e.g. DG44), COS, HEK, a fibroblast cell (e.g., 3T3), and myeloma cells, for example it may be expressed in a CHO or a myeloma cell. Human cells may be used, thus enabling the molecule to be modified with human glycosylation patterns. Alternatively, other eukaryotic cell lines may be employed. The selection of suitable mammalian host cells and methods for transformation, culture, amplification, screening and product production and purification are known in the art. See, e.g., Sambrook et al., cited above.

Bacterial cells may prove useful as host cells suitable for the expression of the recombinant Fabs or other embodiments of the present invention (see, e.g., Plückthun, A., Immunol. Rev., 130:151-188 (1992)). However, due to the tendency of proteins expressed in bacterial cells to be in an unfolded or improperly folded form or in a non-glycosylated form, any recombinant Fab produced in a bacterial cell would have to be screened for retention of antigen binding ability. If the molecule expressed by the bacterial cell was produced in a properly folded form, that bacterial cell would be a desirable host, or in alternative embodiments the molecule may express in the bacterial host and then be subsequently re-folded. For example, various strains of E. coli used for expression are well-known as host cells in the field of biotechnology. Various strains of B. subtilis, Streptomyces, other bacilli and the like may also be employed in this method.

Where desired, strains of yeast cells known to those skilled in the art are also available as host cells, as well as insect cells, e.g. Drosophila and Lepidoptera and viral expression systems. See, e.g. Miller et al., Genetic Engineering, 8:277-298, Plenum Press (1986) and references cited therein.

The general methods by which the vectors may be constructed, the transfection methods required to produce the host cells of the invention, and culture methods necessary to produce the antigen binding protein of the invention from such host cell may all be conventional techniques. Typically, the culture method of the present invention is a serum-free culture method, usually by culturing cells serum-free in suspension. Likewise, once produced, the antigen binding proteins of the invention may be purified from the cell culture contents according to standard procedures of the art, including ammonium sulfate precipitation, affinity columns, column chromatography, gel electrophoresis and the like. Such techniques are within the skill of the art and do not limit this invention. For example, preparation of altered antibodies are described in WO 99/58679 and WO 96/16990.

Yet another method of expression of the antigen binding proteins may utilize expression in a transgenic animal, such as described in U.S. Pat. No. 4,873,316. This relates to an expression system using the animal's casein promoter which when transgenically incorporated into a mammal permits the female to produce the desired recombinant protein in its milk.

In a further aspect of the invention there is provided a method of producing an antibody of the invention which method comprises the step of culturing a host cell transformed or transfected with a vector encoding the light and/or heavy chain of the antibody of the invention and recovering the antibody thereby produced.

In accordance with the present invention there is provided a method of producing an anti-IL-23 antibody of the present invention which binds to and neutralises the activity of human IL-23 which method comprises the steps of;
(a) providing a first vector encoding a heavy chain of the antibody;
(b) providing a second vector encoding a light chain of the antibody;
(c) transforming a mammalian host cell (e.g. CHO) with said first and second vectors;
(d) culturing the host cell of step (c) under conditions conducive to the secretion of the antibody from said host cell into said culture media;
(e) recovering the secreted antibody of step (d).

Once expressed by the desired method, the antibody is then examined for in vitro activity by use of an appropriate assay. Presently conventional ELISA assay formats are employed to assess qualitative and quantitative binding of the antibody to IL-23. Additionally, other in vitro assays may also be used to verify neutralizing efficacy prior to subsequent human clinical studies performed to evaluate the persistence of the antibody in the body despite the usual clearance mechanisms.

The dose and duration of treatment relates to the relative duration of the molecules of the present invention in the human circulation, and can be adjusted by one of skill in the art depending upon the condition being treated and the general health of the patient. It is envisaged that repeated dosing (e.g. once a week or once every two weeks) over an extended time period (e.g. four to six months) maybe required to achieve maximal therapeutic efficacy.

The mode of administration of the therapeutic agent of the invention may be any suitable route which delivers the agent to the host. The antigen binding proteins, and pharmaceutical compositions of the invention are particularly useful for parenteral administration, i.e., subcutaneously (s.c.), intrathecally, intraperitoneally, intramuscularly (i.m.), intravenously (i.v.), or intranasally.

Therapeutic agents of the invention may be prepared as pharmaceutical compositions containing an effective amount of the antigen binding protein of the invention as an active ingredient in a pharmaceutically acceptable carrier. In the prophylactic agent of the invention, an aqueous suspension or solution containing the antigen binding protein, preferably buffered at physiological pH, in a form ready for injection is preferred. The compositions for parenteral administration will commonly comprise a solution of the antigen binding protein of the invention or a cocktail thereof dissolved in a pharmaceutically acceptable carrier, preferably an aqueous carrier. A variety of aqueous carriers may be employed, e.g., 0.9% saline, 0.3% glycine, and the like. These solutions may be made sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antigen binding protein of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g. about 50 ng to about 30 mg or more preferably, about 5 mg to about 25 mg, of an antigen binding protein, for example an antibody of the invention. Similarly, a pharmaceutical composition of the invention for intravenous infusion could be made up to contain about 250 ml of sterile Ringer's solution, and about 1 to about 30 and preferably 5 mg to about 25 mg of an antigen binding protein of the invention per ml of Ringer's solution. Actual methods for preparing parenterally administrable compositions are well known or will be apparent to those skilled in the art and are described in more detail in, for example, Remington's Pharmaceutical Science, 15th ed., Mack Publishing Company, Easton, Pa. For the preparation of intravenously administrable antigen binding protein formulations of the invention see Lasmar U and Parkins D “The formulation of Biopharmaceutical products”, Pharma. Sci. Tech. today, page 129-137, Vol. 3 (3 Apr. 2000), Wang, W “Instability, stabilisation and formulation of liquid protein pharmaceuticals”, Int. J. Pharm 185 (1999) 129-188, Stability of Protein Pharmaceuticals Part A and B ed Ahern T. J., Manning M. C., New York, N.Y.: Plenum Press (1992), Akers, M. J. “Excipient-Drug interactions in Parenteral Formulations”, J. Pharm Sci 91 (2002) 2283-2300, Imamura, K et al “Effects of types of sugar on stabilization of Protein in the dried state”, J Pharm Sci 92 (2003) 266-274, Izutsu, Kkojima, S. “Excipient crystallinity and its protein-structure-stabilizing effect during freeze-drying”, J. Pharm. Pharmacol, 54 (2002) 1033-1039, Johnson, R, “Mannitol-sucrose mixtures-versatile formulations for protein lyophilization”, J. Pharm. Sci, 91 (2002) 914-922.

Ha, E Wang W, Wang Y. j. “Peroxide formation in polysorbate 80 and protein stability”, J. Pharm Sci, 91, 2252-2264, (2002) the entire contents of which are incorporated herein by reference and to which the reader is specifically referred.

It is preferred that the therapeutic agent of the invention, when in a pharmaceutical preparation, be present in unit dose forms. The appropriate therapeutically effective dose will be determined readily by those of skill in the art. Suitable doses may be calculated for patients according to their weight, for example suitable doses may be in the range of 0.1 to 20 mg/kg, for example 1 to 20 mg/kg, for example 10 to 20 mg/kg or for example 1 to 15 mg/kg, for example 10 to 15 mg/kg. To effectively treat conditions such as rheumatoid arthritis, psoriasis, IBD, multiple sclerosis or SLE in a human, suitable doses may be within the range of 0.1 to 1000 mg, for example 0.1 to 500 mg, for example 500 mg, for example 0.1 to 100 mg, or 0.1 to 80 mg, or 0.1 to 60 mg, or 0.1 to 40 mg, or for example 1 to 100 mg, or 1 to 50 mg, of an antigen binding protein of this invention, which may be administered parenterally, for example subcutaneously, intravenously or intramuscularly. Such dose may, if necessary, be repeated at appropriate time intervals selected as appropriate by a physician.

The antigen binding proteins described herein can be lyophilized for storage and reconstituted in a suitable carrier prior to use. This technique has been shown to be effective with conventional immunoglobulins and art-known lyophilization and reconstitution techniques can be employed.

In another aspect, the invention provides a pharmaceutical composition comprising an antigen binding protein of the present invention or a functional fragment thereof and a pharmaceutically acceptable carrier for treatment or prophylaxis of immune system mediated inflammation such as psoriasis, inflammatory bowel disease, ulcerative colitis, crohns disease, rheumatoid arthritis, juvenile rheumatoid arthritis, systemic lupus erythematosus, neurodegenerative diseases, for example multiple sclerosis, neutrophil driven diseases, for example COPD, Wegeners vasculitis, cystic fibrosis, Sjogrens syndrome, chronic transplant rejection, type 1 diabetes graft versus host disease, asthma, allergic diseases for example atoptic dermatitis, eczematous dermatitis, allergic rhinitis, and other autoimmune diseases including thyroiditis, spondyloarthropathy, ankylosing spondylitis, uveitis, polychonritis or scleroderma. In one embodiment the disorder is rheumatoid arthritis.

In a yet further aspect, the invention provides a pharmaceutical composition comprising an antigen binding protein of the present invention and a pharmaceutically acceptable carrier for immune system mediated inflammation such as psoriasis, inflammatory bowel disease, ulcerative colitis, crohns disease, rheumatoid arthritis, juvenile rheumatoid arthritis, systemic lupus erythematosus, neurodegenerative diseases, for example multiple sclerosis, neutrophil driven diseases, for example COPD, Wegenersvasculitis, cystic fibrosis, sjogrens syndrome, chronic transplant, type 1 diabetes graft versus host disease, asthma, allergic diseases for example atoptic dermatitis, eczematous dermatitis, allergic rhinitis, and other autoimmune diseases including thyroiditis, spondyloarthropathy, ankylosing spondylitis, uveitis, polychonritis, or scleroderma. In one embodiment the disorder is rheumatoid arthritis.

It will be understood that the sequences described herein (SEQ ID NO: 8 to SEQ ID NO: 35, SEQ ID NO:48 to SEQ ID NO: 71, SEQ ID NO: 81 to SEQ ID NO: 90, SEQ ID NO: 93, SEQ ID NO: 94, SEQ ID NO:96, SEQ ID NO: 97 and SEQ ID NO: 103 to SEQ ID NO: 123) include sequences which are substantially identical, for example sequences which are at least 90% identical, for example which are at least 91%, or at least 92%, or at least 93%, or at least 94% or at least 95%, or at least 96%, or at least 97% or at least 98%, or at least 99% identical to the sequences described herein.

For nucleic acids, the term “substantial identity” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial identity exists when the segments will hybridize under selective hybridization conditions, to the complement of the strand.

For nucleotide and amino acid sequences, the term “identical” indicates the degree of identity between two nucleic acid or amino acid sequences when optimally aligned and compared with appropriate insertions or deletions. Alternatively, substantial identity exists when the DNA segments will hybridize under selective hybridization conditions, to the complement of the strand.

The percent identity between two sequences is a function of the number of identical positions shared by the sequences (i.e., % identity=# of identical positions/total # of positions times 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package, using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

By way of example, a polynucleotide sequence of the present invention may be identical to the reference sequence of SEQ ID NO: 17, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO: 17 by the numerical percent of the respective percent identity (divided by 100) and subtracting that product from said total number of nucleotides in SEQ ID NO: 17, or:

nn≦xn−(xn·y),

wherein nn is the number of nucleotide alterations, xn is the total number of nucleotides in SEQ ID NO: 17, and y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and wherein any non-integer product of xn and y is rounded down to the nearest integer prior to subtracting it from xn. Alterations of the polynucleotide sequence of SEQ ID NO: 17 may create nonsense, missense or frameshift mutations in this coding sequence and thereby alter the polypeptide encoded by the polynucleotide following such alterations.

Similarly, in another example, a polypeptide sequence of the present invention may be identical to the reference sequence encoded by SEQ ID NO: 16, that is be 100% identical, or it may include up to a certain integer number of amino acid alterations as compared to the reference sequence such that the identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the polypeptide sequence encoded by SEQ ID NO: 16 by the numerical percent of the respective percent identity (divided by 100) and then subtracting that product from said total number of amino acids in the polypeptide sequence encoded by SEQ ID NO: 16, or:

na≦xa−(xa·y),

wherein na is the number of amino acid alterations, xa is the total number of amino acids in the polypeptide sequence encoded by SEQ ID NO: 16, and y is, for instance 0.70 for 70%, 0.80 for 80%, 0.85 for 85% etc., and wherein any non-integer product of xa and y is rounded down to the nearest integer prior to subtracting it from xa.

The following examples illustrate but do not limit the invention.

### EXAMPLES

### Example 1

### Construction of Recombinant Murine, Chimeric and Humanised Anti-IL-23 Antibodies

Murine mAbs were produced by immunisation of mice with human IL-23. Spleens from responder animals were harvested and fused to myeloma cells to generate hybridomas. The hybridoma supernatant material was screened for binding. Hybridomas of interest were monocloned using standard techniques. The murine antibodies (8C9 2H6) which were used in the present examples, when analysed by RT-PCR showed the presence of two heavy chains and one light chain. Both combinations (HC1LC1 and HC2LC1) were constructed in the form of chimeric mAbs. It is believed that the principal active binding domains of the 8C92H6 murine mAbs produced from this hybridoma and which are used in the experiments below comprise the variable regions shown in SEQ ID NO:8 and SEQ ID NO:10.

Chimeric constructs were made by preparing murine V_{H} and V_{L} constructs by RT-PCR with RNA from the mouse hybridoma cell line. RT-PCR products were first cloned into vectors for sequence determination then variable regions were cloned into Rld and Rln mammalian expression vectors using oligonucleotides including restriction sites as well as a human signal sequence (SEQ ID NO:36). These expression vectors contained human constant regions. Alternative constructs were produced using pTT vectors which also included human constant regions.

Humanised V_{H} and V_{L} constructs were prepared de novo by build-up of overlapping oligonucleotides including restriction sites for cloning into Rld and Rln mammalian expression vectors as well as a human signal sequence. Hind III and Spe I restriction sites were introduced to frame the V_{H} domain containing the signal sequence (SEQ ID NO:36) for cloning into Rld containing the human γ1 constant region. Hind III and BsiWI restriction sites were introduced to frame the V_{L} domain containing the signal sequence (SEQ ID NO: 36) for cloning into Rln containing the human kappa constant region. Alternative constructs were produced using pTT vectors which also included human constant regions. Where appropriate, site-directed mutagenesis (SDM) was used to generate different humanised constructs.

### Humanisation:

The mouse light chain variable domain is highly unusual in both sequence and structure due to the absence of a leucine at position 46, and an insertion of 8 amino acids (RSPFGNQL) starting after position 69. A review of the literature and cDNA database identified a single report of a related mouse light chain variable region. In the humanisation of this light chain, leucine at position 46 is absent from the mouse sequence. This motif was transferred over to the humanised light chain.

In the humanisation process a number of changes were made to the mouse sequence. These changes included the following.

A cysteine to serine, alanine or valine substitution was made from the mouse

CDRH3 (SEQ ID NO:3) to the humanised CDRH3 alternative (SEQ ID NO:4, 73, 74).

Additionally a number of alternative CDR sequences were constructed as set out in SEQ ID NO: 72 to 80, SEQ ID NO: 95 and SEQ ID NO: 98 to 102.

### Humanised Heavy Chain A3 (SEQ ID NO: 16)

A suitable framework was selected for CDR grafting, three back mutations were made at positions 27, 30 and 95.

### Humanised Light Chain M0 (SEQ ID NO: 18)

A suitable framework was selected for CDR grafting. A deletion of L46 was made.

### Humanised Light Chain M1 (SEQ ID NO: 20)

A suitable framework was selected for CDR grafting. A deletion of L46 was made. In addition, back mutations were made at positions 59, 64, 68, 69, 70 and there was an insertion of RSPFGNQL between positions 69 and 70.

Humanised Light Chain N1 (SEQ ID NO: 22) A suitable framework was selected for CDR grafting. A deletion of L46 was made. In addition, back mutations at positions 59, 64, 68, 69, 70 and there was an insertion of RSPFGNQL between positions 69 and 70.

### Humanised Light Chain N2 (SEQ ID NO: 24)

A suitable framework was selected for CDR grafting. A deletion of L46 was made. In addition, back mutations were made at positions 59 and 64.

A number of additional humanised variants as set out in SEQ ID NOs: 48, 50, 52, 54, 56, 58, 81 to 90, 96, 97, and 103 to 123 were produced by similar methods.

### Example 2

### Antibody Expression in CHO Cells

Rld and Rln plasmids encoding the heavy and light chains respectively were transiently co-transfected into CHO cells and expressed at small scale or large scale to produce antibody. Alternatively the same plasmids were co-transfected into CHO cells by electroporation and a stable polyclonal population of cells expressing the appropriate antibody were selected using a nucleoside-free media. In some assays, antibodies were assessed directly from the tissue culture supernatant. In other assays, recombinant antibody was recovered and purified by affinity chromatography on Protein A sepharose.

Further details of construction and expression of such antibodies were carried out in accordance with the general methodology described in WO2007/080174 and WO2007/068750.

### Antibody Expression in HEK 293 6E Cells

pTT plasmids encoding the heavy and light chains respectively were transiently co-transfected into HEK 293 6E cells and expressed at small scale or large scale to produce antibody. In some assays, antibodies were assessed directly from the tissue culture supernatant. In other assays, recombinant antibody was recovered and purified by affinity chromatography on Protein A sepharose.

Where we refer to the antibodies by code (i.e. A3M0, A3M1, A3N1, A3N2, HC1LC1) we are referring to the mAb generated by co-transfection and expression of the noted first and second plasmid, for example ‘A3M0’ relates to a mAb generated by co-transfection of the a plasmid containing the A3 sequence and a plasmid containing the M0 sequence in a suitable cell line.

### Example 3

### Biacore Analysis of Murine Anti-IL-23 Antibodies

Anti-murine IgG was immobilised on a CM5 sensorchip using amine coupling chemistry. Anti-IL-23 hybridoma antibody sample was injected over the surface and the murine mAb captured. Subsequently recombinant human IL-23, recombinant cynomologus IL-23 or recombinant human IL-12 was flowed over the captured antibody surface at 5 different concentrations (range 0 nM-91 nM) to obtain binding sensorgrams. Regeneration of the surface after antibody and antigen injections was done by injecting 0.1 M phosphoric acid for 3 minutes. Double referencing was used on all sensorgrams with a buffer injection over the anti-murine IgG sensorchip surface. The experiment was performed at 25° C. in HBS-EP buffer. Resulting sensorgram data was analysed using the 1:1 binding model incorporated within the Biaevaluation software for the Biacore 3000 instrument. Data presented in Table 2 are from using hybridoma supernatant taken from a tissue culture flask.

TABLE 2

| | human IL-23 | | | human IL-12 | | | cynomologus IL-23 | | |
|---|---|---|---|---|---|---|---|---|---|
| Murine | Ka | | | ka | kd | KD | ka | | KD |
| mAb | (1/Ms) | kd (1/s) | KD (nM) | (1/Ms) | (1/s) | (nM) | (1/Ms) | kd (1/s) | (nM) |

| | | | | | | | |
|---|---|---|---|---|---|---|---|
| 8C92H6 | 1.01e5 | 3.01e−4 | 2.99 | no significant | 1.10e5 | 3.69e−4 | 3.38 |
| | | | | binding | | | |

### Example 4

### Binding of Anti-IL-23 Chimeric and Humanised mAbs to Human IL-23

Chimeric and humanised mAbs were evaluated by sandwich ELISA, to determine their binding activity to human IL-23.

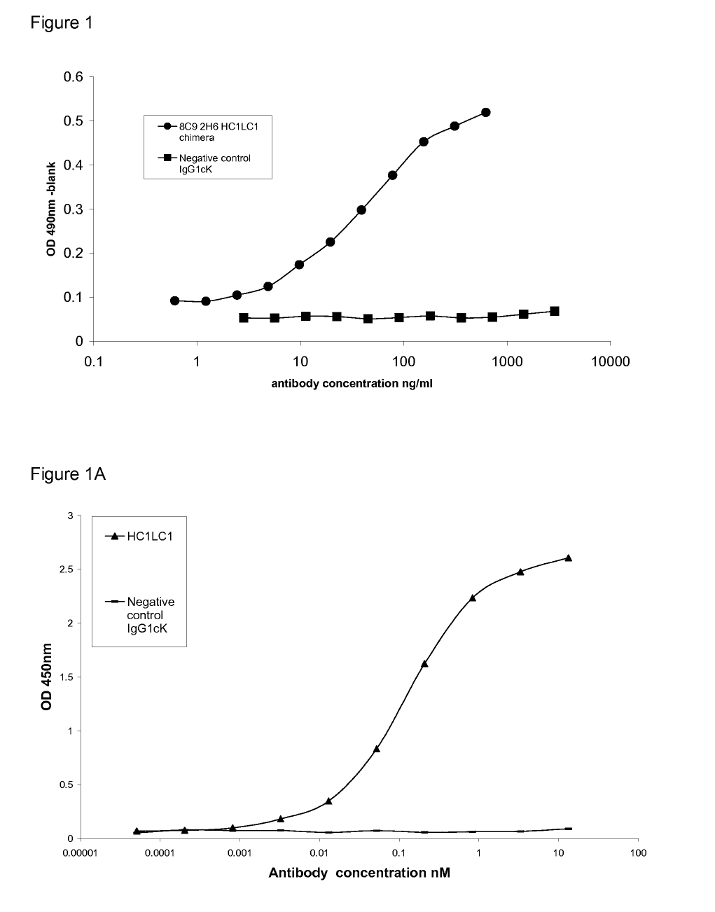

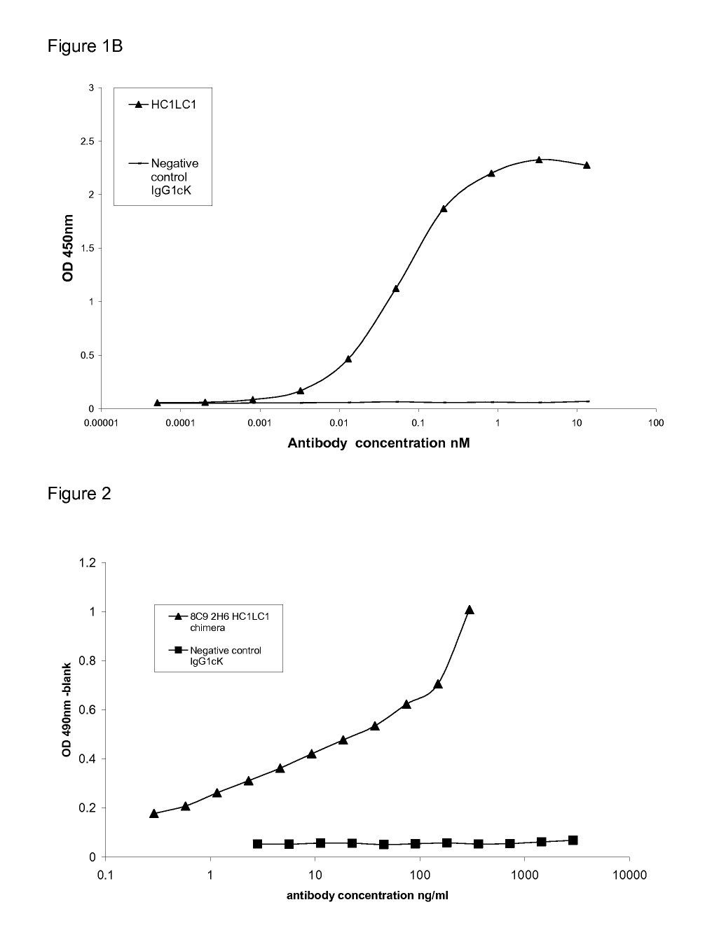

Plates were coated with anti human IL-12 at 1 μg/diluent (bicarbonate buffer). 50 μl/well of this mixture was incubated overnight at 4° C. The plates were then washed twice with Tris Buffered Saline with 0.05% Tween 20 (TBST). Plates were blocked with 1′)/0 BSA TBST 100 μl/well for a minimum of 1 hour at room temperature. The plates were then washed twice with Tris Buffered Saline+0.05% Tween 20 (TBST). Various concentrations of antibody were incubated in a separate plate with a constant concentration of IL-23 for 1 hour at room temperature. 50 ul of each mixture were transferred to the assay plate and incubated at RT for 1 hr. They were then washed twice with Tris Buffered Saline+0.05% Tween 20 (TBST). Bound mAbs were detected by goat anti human IgG gamma chain HRP (Sigma A6029) diluted 1/1000 in 1% BSA TBST. 50 μl/well of the detection antibody was added and incubated at RT for 1 hour. The plates were then washed three times with Tris Buffered Saline+0.05% Tween 20 (TBST). o-phenylenediamine dihydrochloride (OPD) was reconstituted in 20 ml H₂O, 50 μl/well were added and incubated at RT for 20 min. 25 μl/well of 3 MH₂SO₄ was added. The plate was read at OD490 nm using the SOftmaxPRO versamax plate reader. The results are set out in FIGS. 1, 2 and 3.

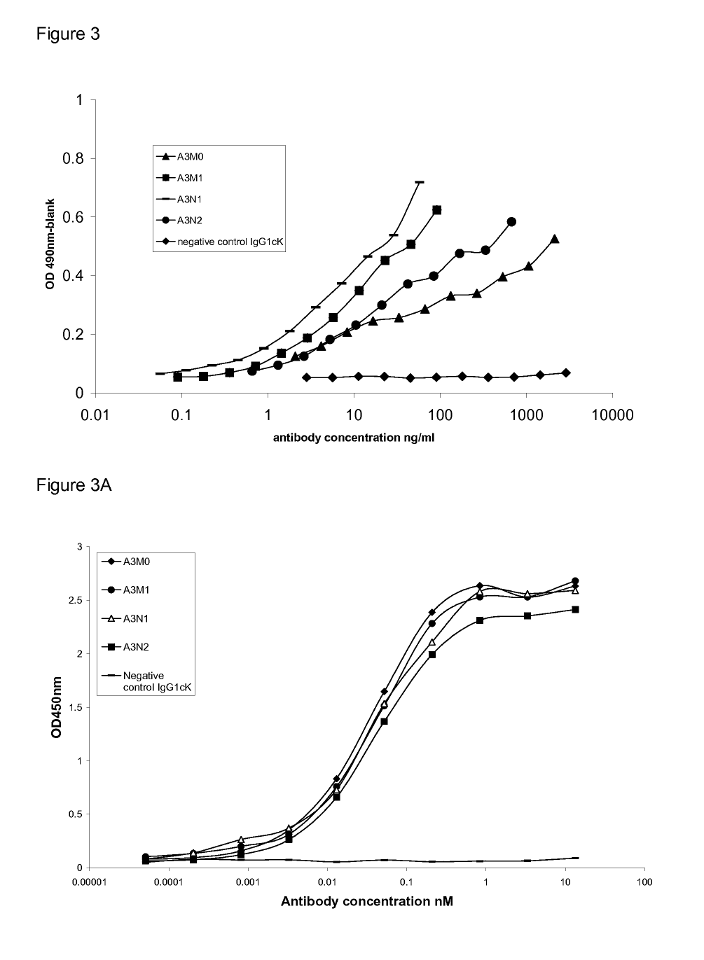

This was repeated using optimised assay conditions as set out below and anti IL-23 antibody material from a different preparation, the results are set out in FIGS. 1A, 1B and 3A. The data shown in FIGS. 1A, 1B and 3A is therefore considered to be more accurate than the data shown in FIGS. 1, 2 and 3. The binding profile of 8C92H6HC1LC1 shown in FIG. 1A differs to that in FIG. 1B, the reason for this difference in binding profile is unknown.

Optimised assay conditions: Plates were coated with anti human IL12 at 2 μg/diluent (phosphate buffered saline). 50 μl/well of this mixture was incubated overnight at 4° C. The plates were then washed three times with Phosphate Buffered Saline with 0.05% Tween 20 (PBST). Plates were blocked with 4% skim milk powder (Fluka BioChemika #70166) PBS 200 μl/well for a minimum of 1 hour at room temperature. The plates were then washed three times with Phosphate Buffered Saline+0.05% Tween 20 (PBST). Various concentrations of antibody were incubated in a separate plate with a constant concentration of IL-23 for 1 hour at room temperature. 50 ul of each mixture were transferred to the assay plate and incubated at RT for 1 hr. They were then washed three times with Phosphate Buffered Saline+0.05% Tween 20 (PBST). Bound mAbs were detected by goat anti human IgG gamma chain HRP (Serotec STAR 106P) diluted 1/3000 in 4% Skim milk powder (Fluka BioChemika #70166) PBS. 50 μl/well of the detection antibody was added and incubated at RT for 1 hour. The plates were then washed three times with Phosphate Buffered Saline+0.05% Tween 20 (PBST). 50 μl/well of TMB was added to the plates and incubated at RT for 10 min. 50 μl/well of 1 MH₂SO₄ was added. The plate was read at OD450 nm using the SOftmaxPRO versamax plate reader.

FIGS. 1, 1A and 1B show the ability of purified chimeric 8C92H6HC1LC1 to bind to human IL-23.

FIG. 2 shows the ability of tissue culture supernatant chimeric 8C92H6 HC1LC1 to bind to human IL-23.

FIG. 3 shows the ability of tissue culture supernatant humanised mAbs to bind to human IL-23.

FIG. 3A show the ability of purified humanised mAbs to bind to human IL-23. All samples were run in duplicate, and averages of each duplicate are shown. In addition, the assays using supernatant material were run twice using different preparations of tissue culture supernatant and a representative result is shown.

### Example 5

### Biacore Analysis of Anti IL-23 Chimeric and Humanised mAbs

Protein A or an anti-human IgG (Biacore BR-1008-39) was immobilised on a Biacore CM5 chip by primary amine coupling in accordance with the manufacturer's instructions. Anti IL-23 antibodies were captured on this surface and after a period of stabilisation, IL13 was passed over the antibody captured surface and a binding sensorgram was obtained. Regeneration was achieved using two pulses of 100 mM phosphoric acid which removed the captured antibody but did not significantly affect the Protein A/anti-human IgG surface's ability to capture antibody in a subsequent binding event. All runs were double referenced with a buffer injection over the captured antibody surface. Data was analysed using the 1:1 model using the software inherent to the Biacore 3000 or T100 depending upon which machine was used to generate kinetics. Analysis was carried out at 25° C. using HBS-EP buffer. Data presented in Tables 3-6 are on tissue culture supernatants of CHO cells transiently expressing the antibody of interest unless otherwise indicated. Data was generated using concentrations of IL-23 (256, 64, 16, 4, 1 and 0.25 nM) The data shown for the humanised variants is representative of a number of runs, The chimeric mAb (8C9H6.HC1LC1) was only run once in each experiment, so data shown for this mAb is from that one run,

TABLE 3

| Construct | ka (1/Ms) | kd (1/s) | KD (nM) |
|---|---|---|---|
| 8C92H6.HC1LC1 | 2.7e5 | 3.9e−4 | 1.4 |
| Chimera | | | |

Data generated on the Biacore 3000 using 3 concentrations of IL-23 (100, 10 and 1 nM)

TABLE 4

| Construct | Ka (1/Ms) | kd (1/s) | KD (nM) |
|---|---|---|---|
| 8C92H6.HC1LC1 | 3.2e5 | 2.9e−4 | 0.91 |
| Chimera | | | |

Data generated on T100 using 10 concentrations of IL-23 (128, 64, 32, 16, 8, 4, 2, 1, 0.5 and 0.25 nM)

TABLE 5

| Construct | ka (1/Ms) | kd (1/s) | KD(nM) |
|---|---|---|---|
| 8C92H6.HC1LC1 | 2.4e5 | 4.4e−4 | 1.8 |
| Chimera (purified) | | | |
| A3M0 | 3.0e5 | 3.3e−4 | 1.1 |
| A3M1 | 2.4e5 | 3.6e−4 | 1.5 |
| A3N1 | 1.7e5 | 3.9e−4 | 2.3 |
| A3N2 | 2.8e5 | 4.1e−4 | 1.5 |

Data generated on Biacore 3000 using 4 concentrations of IL-23 (256, 64, 16 and 4 nM).

TABLE 6

| Construct | ka (1/Ms) | kd (1/s) | KD(nM) |
|---|---|---|---|
| A3M0 | 3e5 | 2.8e−4 | 0.92 |
| A3M1 | 1.3e5 | 3.1e−4 | 2.4 |
| A3N1 | 8.4e4 | 3.5e−4 | 4.1 |
| A3N2 | 2.4e5 | 3.8e−4 | 1.6 |
| 8C92H6.HC1LC1 | 2.1e5 | 3.8e−4 | 1.8 |
| Chimera (transient | | | |
| material) | | | |
| 8C92H6.HC1LC1 | 2.0e5 | 4.3e−4 | 2.2 |
| Chimera (purified | | | |
| material) | | | |

Data generated on the T100 using 5 concentrations of IL-23 (256, 64, 16, 4 and 1 nM)

### Example 5A

### Biacore Analysis of Purified Chimeric and Humanised mAbs

This is essentially a repeat of Example 5 but using a different source of IL-23.

Biacore analysis was carried out using a capture surface on a CM5 chip. Anti-human IgG (BR-1008-39) was used as the capturing agent. Anti-human IgG was coupled to a CM5 biosensor chip by primary amine coupling. Humanised antibody was captured on this immobilised surface and defined concentrations of IL-23 were passed over this captured surface. An injection of buffer over the captured antibody surface was used for double-referencing. The captured surface was regenerated, after each IL-23 injection using 3M MgCl2, the regeneration removed the captured antibody but did not significantly affect the ability of the surface to capture antibody in a subsequent cycle. T100 Biacore machine was used to generate the data; all runs were carried out at 25° C. using HBS EP. Data was analysed using the software inherent to the machine and fitted to the 1:1 model of binding. Tables 7 and 8 detail the IL-23 binding analysis carried out on the 8C92H₆.HC1LC1 chimera and selected humanised variants in two separate experiments. Data presented in Tables 7 and 8 are from purified antibody samples. The data shown for the humanised variants is representative of a number of runs. The chimeric mAb (8C9H₆.HC1LC1) was only run once in each experiment, so data shown for this mAb is from that one run. Table 8A shows data from tissue culture supernatants from the same Biacore run including A3M0 for comparison purposes.

TABLE 7

| | ka (M−1 · s−1) | Kd (s−1) | KD (pM) |
|---|---|---|---|
| 8C92H6.HC1LC1. Chimera | 9.25e+5 | 3.37e−4 | 364 |
| A3M0 | 1.27e+6 | 2.40e−4 | 190 |

TABLE 8

| | ka (M−1 · s−1) | kd (s−1) | KD (pM) |
|---|---|---|---|
| A3M0 | 1.22E+6 | 2.37E−4 | 194 |
| A7M3 | 1.01E+6 | 1.45E−4 | 144 |
| A6M0 | 2.95E+6 | 2.98E−4 | 101 |
| A9M3 | 2.64E+6 | 1.71E−4 | 65 |
| A5M0 | 1.65E+6 | 1.71E−4 | 103 |
| A8M3 | 1.67E+6 | 1.35E−4 | 80 |

TABLE 8A

| | Ka (M−1 · s−1) | Kd (s−1) | KD (pM) |
|---|---|---|---|
| A3M0 | 1.38E+6 | 2.34E−4 | 170.3 |
| A3M4 | 1.36E+6 | 1.06E−4 | 77.5 |
| A3M5 | 4.55E+4 | 1.20E−3 | 26400 (26.4 nM) |
| A3M6 | 8.96E+5 | 1.10E−3 | 1230 |
| A10.5M3 | 1.19E+6 | 1.15E−4 | 96.2 |
| A11.5M3 | 1.55E+6 | 9.38E−5 | 60.6 |
| A12.5M3 | 2.70E+6 | 1.72E−4 | 63.7 |
| A5M4 | 1.91E+6 | 1.01E−4 | 53.0 |
| A6M4 | 3.22E+6 | 1.49E−4 | 46.5 |
| A7M4 | 1.34E+6 | 1.23E−4 | 91.8 |
| A8M4 | 2.08E+6 | 9.58E−5 | 46.2 |
| A9M4 | 2.83E+6 | 1.29E−4 | 45.6 |
| A10M4 | 1.46E+6 | 1.07E−4 | 73.3 |
| A11M4 | 2.07E+6 | 9.18E−5 | 44.4 |
| A12M4 | 2.94E+6 | 1.63E−4 | 55.3 |
| A10.5M4 | 2.08E+6 | 8.64E−5 | 41.6 |
| A11.5M4 | 1.45E+6 | 9.62E−5 | 66.5 |
| A12.5M4 | 3.12E+6 | 1.51E−4 | 48.3 |
| A10M3 | 1.11E+6 | 1.17E−4 | 105.6 |
| A11M3 | 1.43E+6 | 9.57E−5 | 67.1 |
| A12M3 | 2.54E+6 | 2.02E−4 | 79.4 |

### Example 6

### Inhibition of IL-23 Binding to IL-23 Receptor in the Presence of Anti-IL-23 mAbs (Murine, Chimeric and Humanised)

In order to demonstrate that the anti-IL-23 mAbs are IL-23 specific neutralising antibodies, the murine mAb was tested for preferential inhibition of binding of IL-23 to IL-23 receptor over inhibition of IL-12 (or IL-23) to IL-12Rβ1.

Anti-IL-23 murine 8C92H₆ mAb was tested in the following assay. Recombinant human IL-23 Receptor (R&D systems 1400-IR-050) or IL-12Rβ1 (R&D systems 839-B1-100) or IL-12Rβ2 (R&D systems 1959-B2-050) was coated onto 96 well plates at a concentration of 1 μg/ml when using single receptors on the plate. When combining both IL-12Rβ1 and β2 both were diluted to 0.5 μg/ml before coating onto plates. Plates were washed with PBS containing 0.05% Tween 20 and then blocked with PBS containing 1% BSA. Human or cynomologus IL-23 or human IL-12 (R&D systems 219-IL-025) at 50 μg/ml, was pre incubated for 1 hour with an equal volume of titrated purified antibody material before being added to the pre-coated plates. Detection was performed with biotinylated anti-human IL12 (R&D systems BAF-219) followed by Streptavidin-HRP (GE Healthcare RPN 4401).

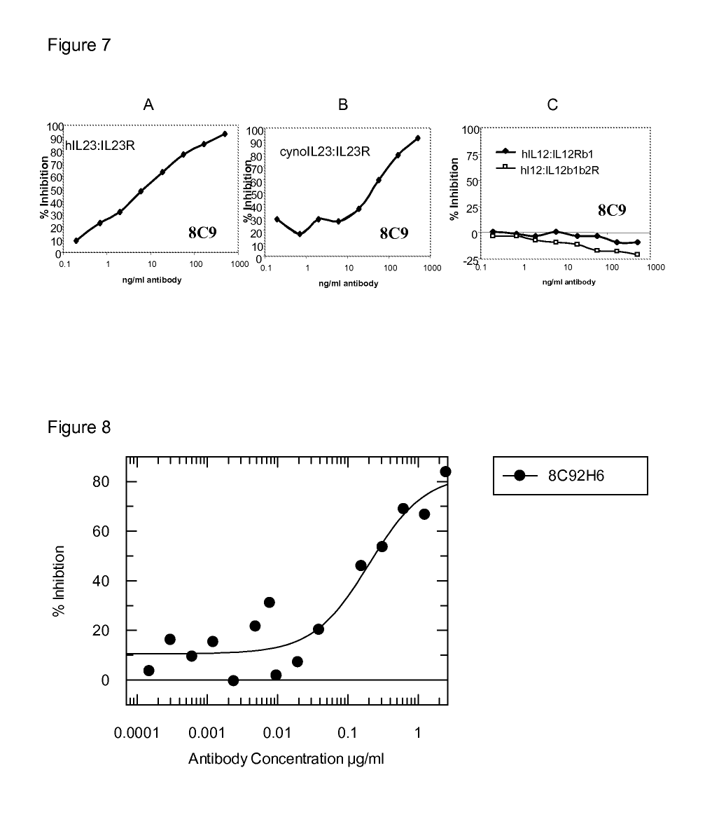

As shown in FIG. 7, IL-23 murine 8C92H₆ mAb, is able to inhibit the binding of human IL-23 (FIG. 7A) and inhibit the binding of cynomolgus IL-23 to IL-23 receptor (FIG. 7B). In contrast to this, anti-IL-23 mAb did not inhibit the binding of recombinant human IL-12 to either IL12Rβ1 alone or a combination of IL12Rβ1 and IL12Rβ2 (FIG. 7C). Data represents the % inhibition of binding of IL-23 to IL-23R in conditions treated with neutralising mAb compared to an irrelevant control IgG (0% inhibition).

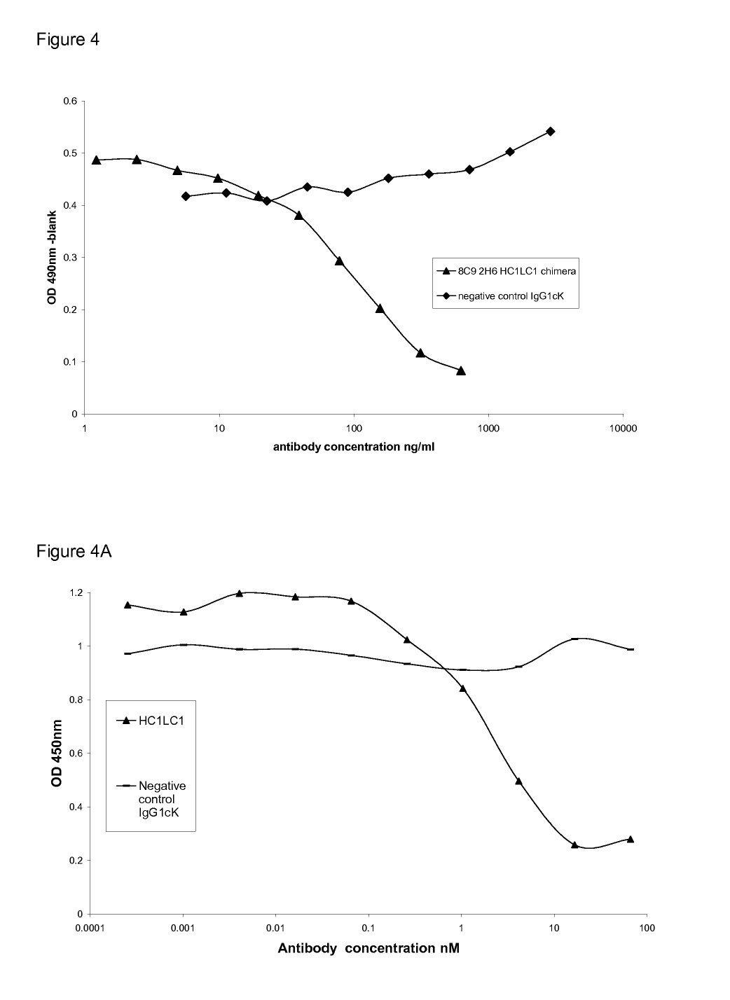

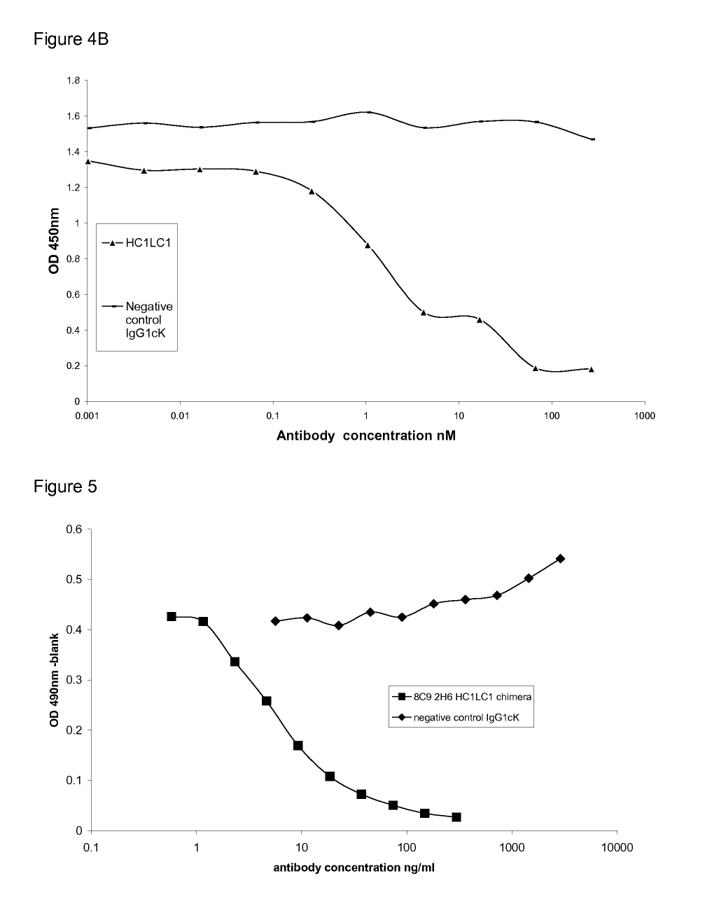

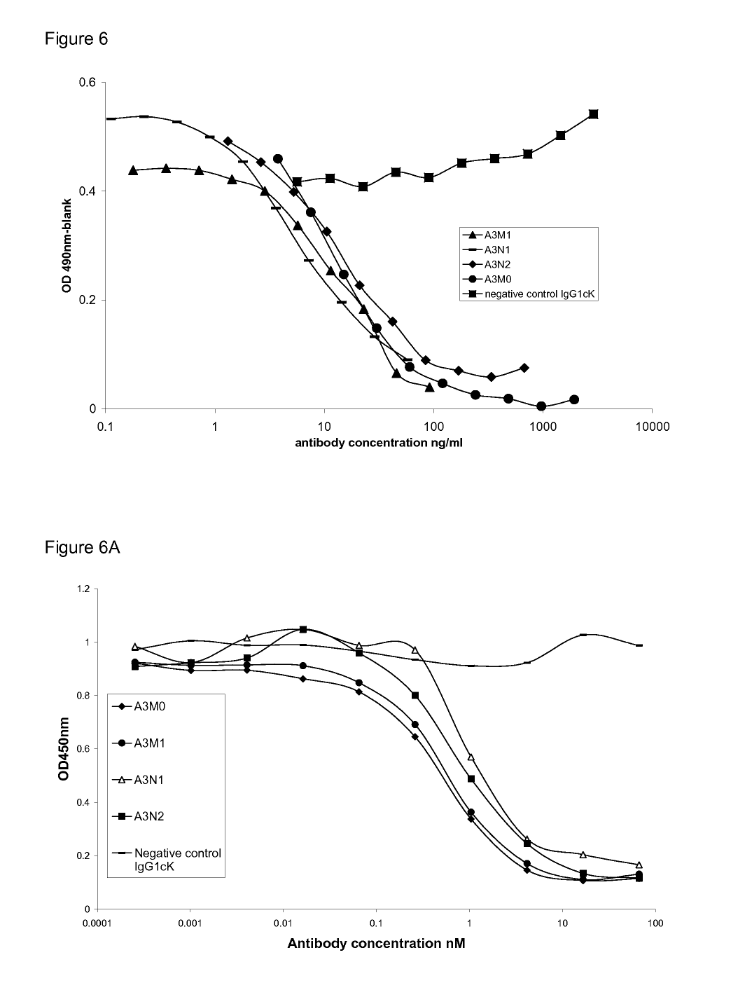

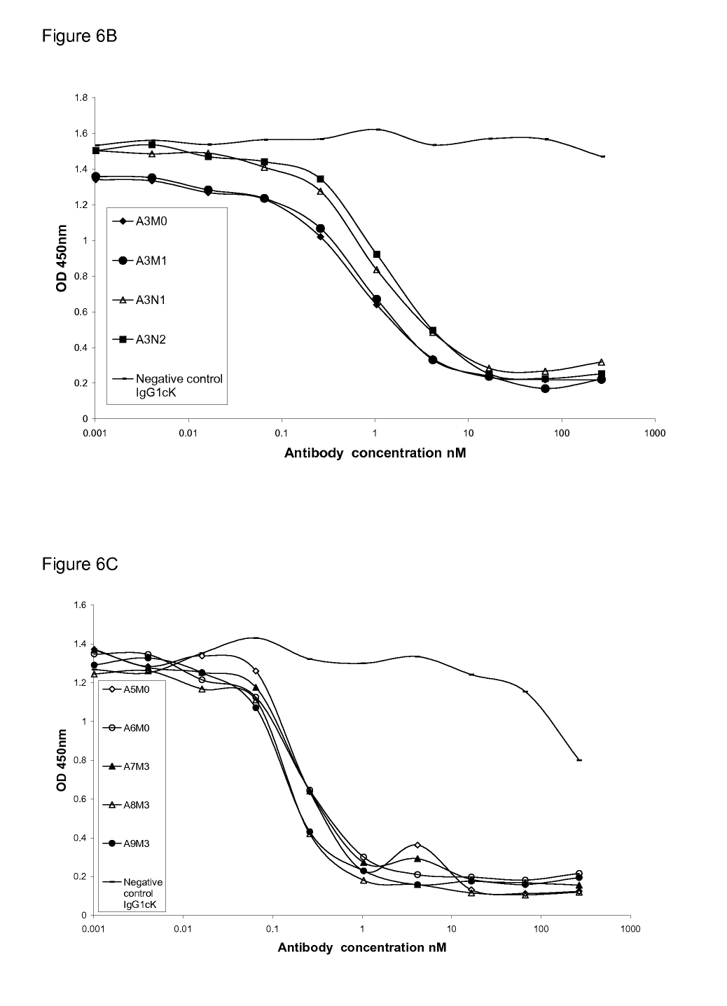

Chimeric and humanised mAbs were assessed for their ability to neutralise human IL-23 binding to human IL-23 receptor, and cyno IL23 binding to human IL23 receptor. Plates were coated with human IL23R Fc chimera at 1 μg/diluent (bicarbonate buffer). 50 μl/well of this mixture was incubated overnight at 4° C. The plates were then washed twice with Tris Buffered Saline with 0.05% Tween 20 (TBST). Plates were blocked with 1% BSA TBST 100 μl/well for a minimum of 1 hour at room temperature. The plates were then washed twice with Tris Buffered Saline with 0.05% Tween 20 (TBST). Various concentrations of antibody were incubated in a separate plate with a constant concentration of IL-23 for 1 hour at room temperature. 50 ul of each mixture were transferred to the assay plate and incubated at RT for 1 hr. They were then washed twice with Tris Buffered Saline with 0.05% Tween 20 (TBST). Bound IL23 was detected by anti human IL12 Biotin labelled Ab (R&D systems BAF219) diluted to 100 ng/ml in 1% BSA TBST. 50 μl/well of the biotinylated antibody was added and incubated at RT for 1 hour. The plates were then washed twice with Tris Buffered Saline with 0.05% Tween 20 (TBST). ExtrAvidin-Peroxidase (Sigma E2886) was diluted 1/1000 in 1% BSA TBST, 50 μl/well was added to the plates. The plates were then washed three times with Tris Buffered Saline with 0.05% Tween 20 (TBST). 50 ul/well of OPD reconstituted in H₂O (Sigma P9187) was added to the plates and incubated at RT for 20 min. 25 μl/well of 3 MH₂SO₄ was added to the wells already containing OPD. The plate was read at OD490 nm using the SOftmaxPRO versamax plate reader. The results are shown in FIGS. 4, 5 and 6. This was repeated using optimised assay conditions as set out below and anti IL-23 antibody material from a different preparation to that used in the earlier assay, and the results are set out in FIGS. 4A and 4B, and FIGS. 6A, 6B and 6C. The data shown in FIGS. 4A and 4B and FIGS. 6A, 6B and 6C is therefore considered to be more accurate than the data shown in FIGS. 4, 5 and 6.

Optimised protocol: Plates were coated with human IL23R Fc chimera at 1 μg/diluent (phosphate buffered saline). 50 μl/well of this mixture was incubated overnight at 4° C. The plates were then washed three times with Phosphate Buffered Saline with 0.05% Tween 20 (PBST). Plates were blocked with 4% skim milk powder (Fluka BioChemika #70166) PBST 100 μl/well for a minimum of 1 hour at room temperature. The plates were then washed three times with Phosphate Buffered Saline+0.05% Tween 20 (PBST). Various concentrations of antibody were incubated in a separate plate with a constant concentration of IL-23 for 1 hour at room temperature. 50 ul of each mixture were transferred to the assay plate and incubated at RT for 1 hr. They were then washed three times with Phosphate Buffered Saline+0.05% Tween 20 (PBST). Bound IL23 was detected by anti human IL12 Biotin labelled Ab (R&D systems BAF219) diluted to 100 ng/ml in 4% skim milk powder (Fluka BioChemika #70166) PBST. 50 μl/well of the biotinylated antibody was added and incubated at RT for 1 hour. The plates were then washed three times with Phosphate Buffered Saline+0.05% Tween 20 (PBST). SA HRP (GE healthcare RPN4401) was diluted 1/4000 in 4% skim milk powder (Fluka BioChemika #70166) PBS, 50 μl/well was added to the plates. The plates were then washed three times with Phosphate Buffered Saline+0.05% Tween 20 (PBST). 50 ul/well of TMB was added to the plates and incubated at RT for 15 min. 25 μl/well of 3 MH₂SO₄ was added to the wells already containing TMB. The plate was read at OD450 nm using the SOftmaxPRO versamax plate reader.

FIGS. 4 and 4A show the ability of purified chimeric 8C92H6HC1LC1 to inhibit human IL-23 binding to human IL-23R.

FIG. 4B show the ability of purified chimeric 8C92H6HC1LC1 to inhibit cynomolgus IL-23 binding to human IL-23R.

FIG. 5 shows the ability of tissue culture supernatant containing chimeric 8C92H6HC1LC1 to inhibit human IL-23 binding to human IL-23R.

FIG. 6 shows the ability of tissue culture supernatant humanised mAbs to inhibit binding of human IL-23 to human IL-23R

FIGS. 6A and 6C show the ability of purified humanised mAbs to inhibit binding of human IL-23 to human IL-23R

FIG. 6B shows the ability of purified humanised mAbs to inhibit binding of cynomolgus IL-23 to human IL-23R

All samples were run in duplicate, and averages of each duplicate are shown. In addition, the assays using supernatant material were run twice using different preparations of tissue culture supernatant and a representative result is shown.

Humanised antibodies A3M4, A5M4, A6M4, A7M4, A8M4, A9M4, A10M4, A11M4, A12M4, A10.5M4, A11.5M4, A12.5M4, A10.5M3, A11.5M3, A12.5M3, A10M3, A11M3, and A12M3 in tissue culture supernatant were also tested in this assay. All of these antibodies neutralised binding of human IL23 to human IL23R, the 1050 values were in the range of 0.14 nM to 0.57 nM (data not shown).

### Example 7

### Inhibition of IL-23 Biological Activity by Anti-IL-23 Murine and Humanised mAbs

Freshly isolated murine splenocytes were treated with recombinant human IL-23 either alone or following pre-incubation with titrated IL-23 mAbs. After 3 days of culture cell supernatants were collected and assayed by ELISA using IL-17 or IL-22 ELISA duo set (R&D systems).

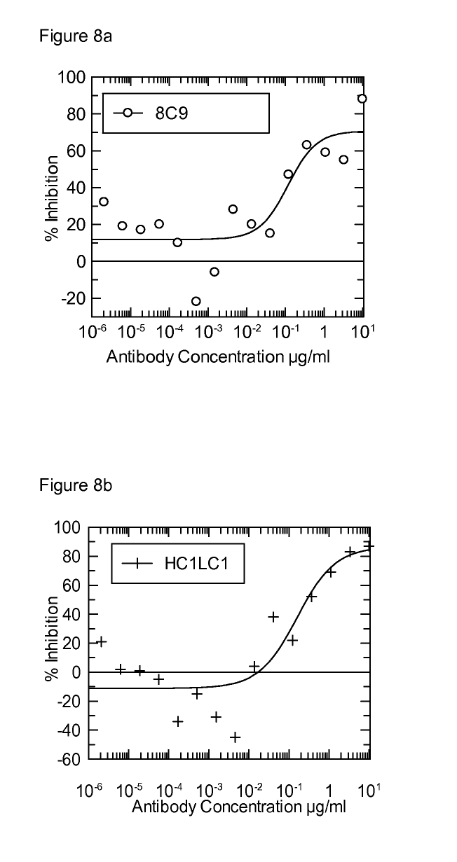

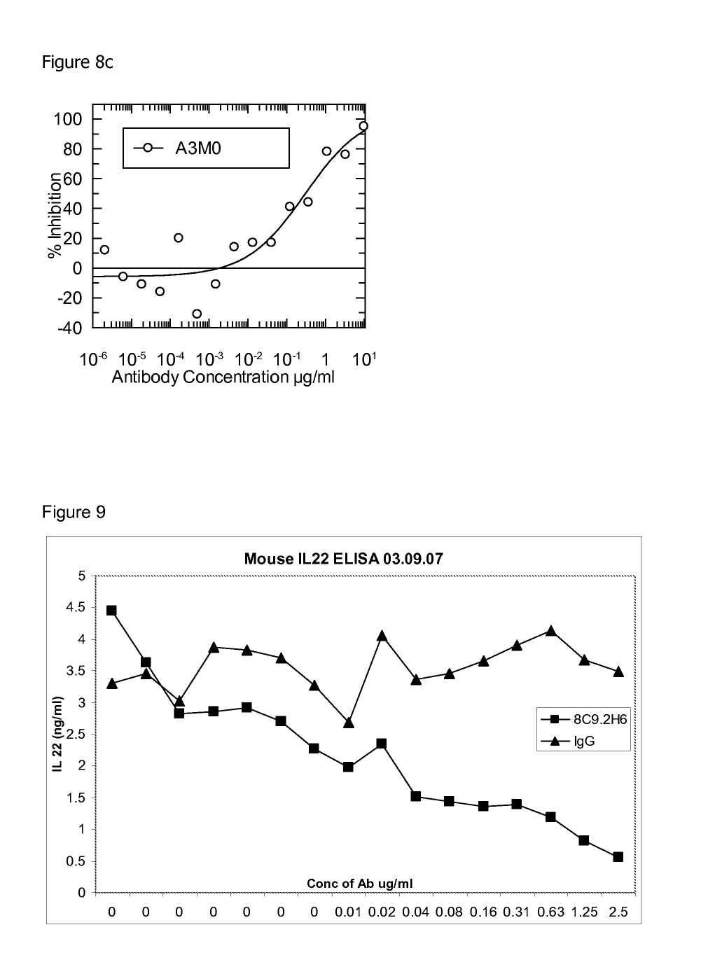

The ability of anti-IL-23 mAbs to inhibit the production of murine IL-17 from splenocytes following incubation with human recombinant IL-23 is shown in FIGS. 8, 8A, 8B and 8C.

The murine antibody was tested for inhibition with three different sources of IL-23. One example is shown in FIG. 8. In a further experiment, the murine mAb was compared with the chimeric antibody and a humanised variant (A3M0) as shown in FIGS. 8A-C. The antibodies inhibited the production of murine IL-17 from splenocytes following incubation with human recombinant IL-23.

Data (plotted using Grafit) represents the % inhibition obtained with neutralising mAbs compared to the levels of IL-17 produced by conditions that included an irrelevant IgG (i.e. 0% inhibition).

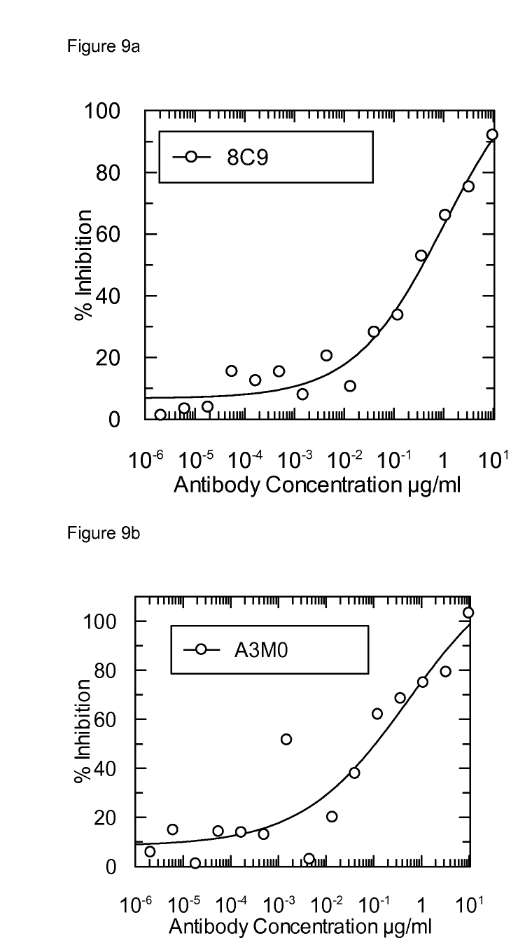

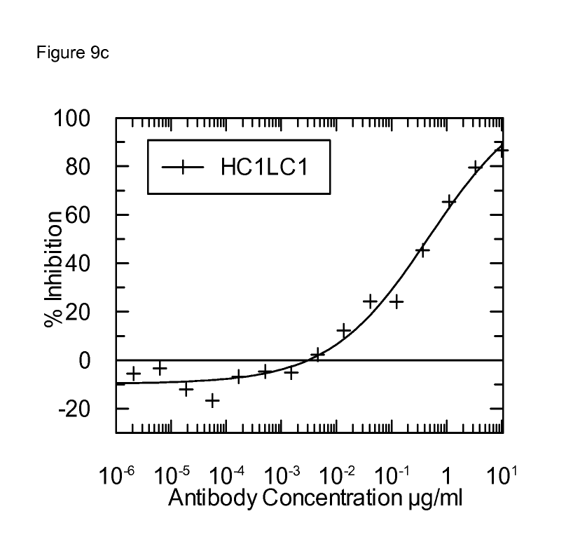

FIGS. 9, 9A, 9B and 9C show the ability of anti-IL-23 mAbs to inhibit the IL-23 driven IL-22 production from murine splenocytes.

FIG. 9 shows the measured amount of IL-22 in the splenocytes when incubated with murine antibody or control IgG.

FIGS. 9A-C show % inhibition of IL-22 production in this assay. FIG. 9A represents the murine antibody, 9B represents humanised antibody A3M0, and 9C represents the chimeric antibody.

### Example 8

### Comparison Between Anti-IL-23 mAbs and Anti-IL-12/23 p40 mAbs on Their Ability to Inhibit IL-12 Induced IFNγ Production from NK92 Cells

The natural killer cell line, NK92 (ATCC# CRL-2407) was propagated according to the ATCC guidelines. This cell line secretes IFNγ in response to IL-12 in a dose-dependant manner. Cells, 4×10⁴ per well, were cultured for 3 days in the presence of media or 1 ng of IL-12 (Peprotech) alone or with IL-12 that had been pre-incubated with a titration of purified antibody material for 1 h at room temperature before being added to the cells. Cell culture supernatants were harvested and analysed after 3 d of culture and the IFNγ content quantified using anti-hulFNγ antibody pairs (Biosource) according to manufacturer's instructions. Briefly, anti-human IFNγ capture mAb was coated onto 96 well flat bottomed Nunc Maxisorp™ plates. Plates were blocked with 1′)/0 BSA before the addition of samples. Detection was performed with biotinylated detection mAb (Biosource) followed by streptavidin-HRP and TMB substrate. Values obtained with IL-12 alone was used as a positive control, media alone as a negative control.

Anti-IL23 mAbs of the present invention had no effect on the production of IL-12-driven IFNγ production from NK92 cells (see FIG. 10). This demonstrates that the anti-IL23 mAbs of the present invention do not inhibit the binding of IL-12 to its receptors and therefore suggests that this antibody recognises an epitope that is not shared between IL12 and IL-23.

### Example 9

### Inhibition of endogenous human IL-23 binding to IL-23 receptor by anti-IL-23 mAbs (murine, chimeric and humanised) 8C92H6 mouse parental, chimeric antibody HC1LC1, and humanised variant A3M0 were assessed for their ability to neutralize endogenous human IL-23 binding to human IL-23 Receptor.

Endogenous human IL-23 was prepared from stimulated dendritic cells. Briefly, monocytes purified by negative selection from peripheral blood mononuclear cells were cultured for 5 days in the presence of GMCSF/IL-4. After this time cells were washed and stimulated with CD40L and zymosan. After a further 24 hours supernatants were removed from the cells and stored before assessment of IL-23 content (ELISA) and use in receptor neutralisation assays.

Recombinant human IL-23 Receptor (R&D systems 1400-IR-050) was coated onto 96 well plates at a concentration of 1 μg/ml. Endogenous human IL-23 at 3.5 ng/ml final, was pre incubated for 1 hour with a titration of purified antibody material before being added to the pre-coated plates. Detection was performed with biotinylated anti-human IL12 (R&D systems BAF-219) followed by Streptavidin-HRP (GE Healthcare RPN 4401). This neutralisation ELISA used 1% BSA.

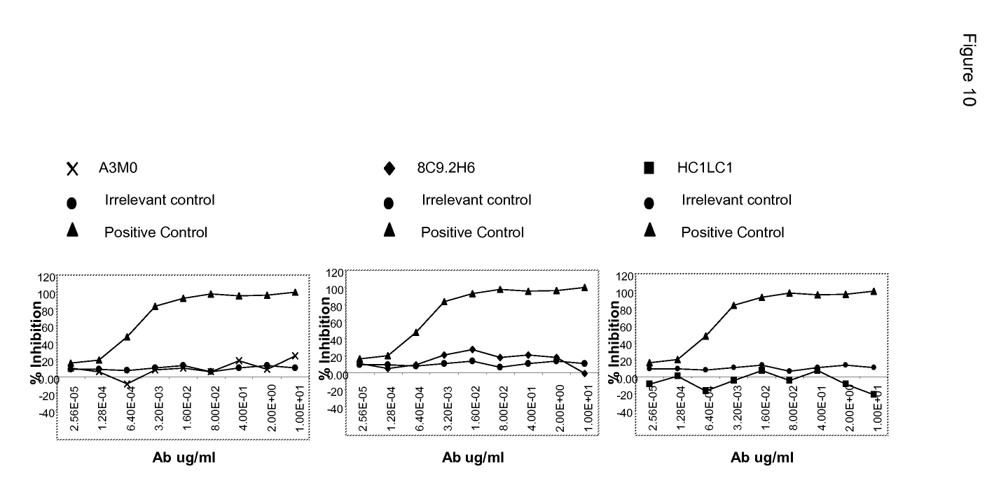

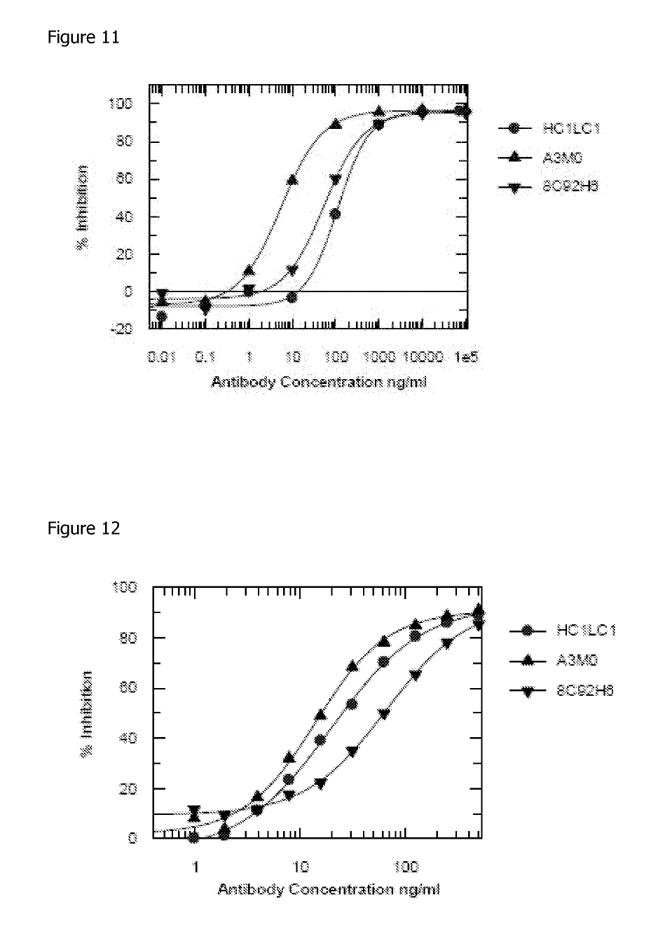

Murine mAb (8C92H₆), chimeric mAb (HC1LC1), and humanised mAb (A3M0) neutralised endogenous human IL-23 and inhibited binding of human IL-23 to human IL-23 receptor. Representative data is shown in FIG. 11.

### Example 10

### Inhibition of Endogenous Human IL-23 Binding to IL-23 Receptor in the Presence of 25% AB Serum by Anti-IL-23 mAbs

Recombinant human IL-23 Receptor (R&D systems 1400-IR-050) was coated onto 96 well plates at a concentration of 1 μg/ml. Endogenous human IL-23 at 5 ng/ml final, was pre incubated with a titration of purified mAbs before being added to the pre-coated plates. Detection was performed with biotinylated anti-human IL12 (R&D systems BAF-219), followed by Streptavidin-HRP (GE Healthcare RPN 4401). This neutralisation ELISA used 25% human pooled AB type serum. 8C92H₆, HC1LC1, and A3M0 retain their activity in human serum and inhibit binding of endogenous human IL-23 binding to human IL-23 receptor. Representative data is shown in FIG. 12.

### Example 11

### Inhibition of IL-23 Driven pSTAT3 Signalling Via the Endogenous Receptor Complex in Human Cells by Anti-IL-23 mAbs

IL-23 driven pSTAT3 signalling via the endogenous receptor complex is measured in this assay by the quantification of the phosphorylation of STAT3 in the DB human lymphoma cell line (ATCC CCRL-2289). This cell line was identified by screening cell lines for IL-23R and IL12β1 expression at the mRNA level (Taqman) and cell surface receptor expression (flow cytometry, data not shown). DB cells respond to human IL-23 in a dose dependent manner as monitored by STAT3 phosphorylation.

Human IL-23 (R&D systems 1290-IL) 50 ng/ml was pre-incubated with various concentrations of purified antibody material for 30 minutes at room temperature. The IL-23/antibody mix was then added to 1.25×10⁶ DB cells for 10 minutes at room temperature, then the cells were harvested and lysed on ice in lysis buffer (Cell Signaling) at a final concentration of 1×. The expression of phospho-STAT3 in these lysates was quantified by immunoassay (Mesoscale Discovery kit K110-DID2). The IC₅₀ values represent data for 3 biological replicates, assayed in 3 independent experiments. The IC₅₀ values for A5M0, A6M0, A7M3, A8M3 and A9M3 represent data for 3 biological replicates, assayed in 2 independent experiments.

IC₅₀ values were determined for the parental antibody 8C92H6, the chimeric antibody HC1LC1, the humanized antibodies A3M0 A5M0, A6M0, A7M3, A8M3 and A9M3. Data presented are the mean IC₅₀ from independent assays (Table 9) which were calculated using Grafit. All antibodies inhibited phosphorylation of STAT3 induced by IL-23. The negative control mAb had no effect on the levels of phosphorylated STAT3 in this assay (data not shown).

TABLE 9

| | IC₅₀ value (+/−standard error) |
|---|---|
| 8C92H6 | 231.67 ng/ml ± 14.57 |
| (mouse parental) | 1.545 nM ± 0.097 |
| HC1LC1 | 93.55 ng/ml ± 4.33 |
| (8C9 chimera) | 0.624 nM ± 0.029 |
| A3M0 | 43.93 ng/ml ± 7.33 |
| (humanised) | 0.287 nM ± 0.049 |
| A5M0 | 22.27 ng/ml ± 13.18 |
| | 0.148 nM ± 0.086 |
| A6M0 | 21.44 ng/ml ± 13.53 |
| | 0.143 nM ± 0.09 |
| A7M3 | 45.85 ng/ml ± 16.76 |
| | 0.306 nM ± 0.11 |
| A8M3 | 36.10 ng/ml ± 11.48 |
| | 0.241 nM ± 0.077 |
| A9M3 | 27.15 ng/ml ± 17.18 |
| | 0.181 nM ± 0.11 |

Sequence Summary (Table 10)

| | Sequence identifier | |
|---|---|---|
| | (SEQ. I.D. NO) | |
| | amino acid | Polynucleotide |
| Description | sequence | sequence |
| 8C9 2H6, CDRH1 | 1 | — |
| 8C9 2H6, CDRH2 | 2 | — |
| 8C9 2H6, CDRH3 | 3 | — |
| CDRH3 alternative | 4 | |
| 8C9 2H6, CDRL1 | 5 | — |
| 8C9 2H6, CDRL2 | 6 | — |
| 8C9 2H6, CDRL3 | 7 | — |
| 8C9 2H6, VH (murine) | 8 | 9 |
| 8C9 2H6, VL (murine) | 10 | 11 |
| Chimeric heavy chain HC1 | 12 | 13 |
| Chimeric light chain LC1 | 14 | 15 |
| 8C9 2H6 VH humanised construct A3 | 16 | 17 |
| 8C9 2H6 VL humanised construct M0 | 18 | 19 |
| 8C9 2H6 VL humanised construct M1 | 20 | 21 |
| 8C9 2H6 VL humanised construct N1 | 22 | 23 |
| 8C9 2H6 VL humanised construct N2 | 24 | 25 |
| 8C9 2H6 heavy chain humanised construct | 26 | 27 |
| A3 | | |
| 8C9 2H6 light chain humanised | 28 | 29 |
| construct M0 | | |
| 8C9 2H6 light chain humanised | 30 | 31 |
| construct M1 | | |
| 8C9 2H6 light chain humanised | 32 | 33 |
| construct N1 | | |
| 8C9 2H6 light chain humanised | 34 | 35 |
| construct N2 | | |
| Signal sequence | 36 | — |
| Human p19 | 37 | 38 |
| Human p40 | 39 | 40 |
| Human p35 | 41 | 42 |
| Cyno p19 | 43 | 44 |
| Cyno p40 | 45 | 46 |
| IL-23 receptor | 47 | — |
| 8C9 2H6 VH humanised construct A5 | 48 | 49 |
| 8C9 2H6 VH humanised construct A6 | 50 | 51 |
| 8C9 2H6 VH humanised construct A7 | 52 | 53 |
| 8C9 2H6 VH humanised construct A10 | 54 | 55 |
| 8C9 2H6 VL humanised construct M3 | 56 | 57 |
| 8C9 2H6 VL humanised construct M4 | 58 | 59 |
| 8C9 2H6 heavy chain humanised | 60 | 61 |
| construct A5 | | |
| 8C9 2H6 heavy chain humanised | 62 | 63 |
| construct A6 | | |
| 8C9 2H6 heavy chain humanised | 64 | 65 |
| construct A7 | | |
| 8C9 2H6 heavy chain humanised | 66 | 67 |
| construct A10 | | |
| 8C9 2H6 light chain humanised | 68 | 69 |
| construct M3 | | |
| 8C9 2H6 light chain humanised | 70 | 71 |
| construct M4 | | |
| CDRH2 alternative | 72 | |
| CDRH3 alternative | 73 | |
| CDRH3 alternative | 74 | |
| CDRL1 alternative | 75 | |
| CDRL2 alternative | 76 | |
| CDRL2 alternative | 77 | |
| CDRL2 alternative | 78 | |
| CDRL2 alternative | 79 | |
| CDRL2 alternative | 80 | |
| 8C9 2H6 VH humanised construct A8 | 81 | |
| 8C9 2H6 VH humanised construct A9 | 82 | |
| 8C9 2H6 VH humanised construct A11 | 83 | |
| 8C9 2H6 VH humanised construct A12 | 84 | |
| 8C9 2H6 VH humanised construct A10.5 | 85 | |
| 8C9 2H6 VH humanised construct A11.5 | 86 | |
| 8C9 2H6 VH humanised construct A12.5 | 87 | |
| 8C9 2H6 VH humanised construct A13 | 88 | |
| 8C9 2H6 VH humanised construct A14 | 89 | |
| 8C9 2H6 VH humanised construct A15 | 90 | |
| Human kappa chain constant region | 91 | |
| Human IgG1 constant region | 92 | |
| 8C9 2H6 light chain humanised | 93 | |
| construct M5 | | |
| 8C9 2H6 light chain humanised | 94 | |
| construct M6 | | |
| CDRH3 alternative | 95 | |
| 8C9 2H6 VL humanised construct M5 | 96 | |
| 8C9 2H6 VL humanised construct M6 | 97 | |
| CDRH2 alternative | 98 | |
| CDRH2 alternative | 99 | |
| CDRH3 alternative | 100 | |
| CDRL1 alternative | 101 | |
| CDRL2 alternative | 102 | |
| 8C9 2H6 VH humanised construct A16 | 103 | |
| 8C9 2H6 VH humanised construct A17 | 104 | |
| 8C9 2H6 VH humanised construct A18 | 105 | |
| 8C9 2H6 VH humanised construct A19 | 106 | |
| 8C9 2H6 VH humanised construct A20 | 107 | |
| 8C9 2H6 VH humanised construct A21 | 108 | |
| 8C9 2H6 VH humanised construct A22 | 109 | |
| 8C9 2H6 VH humanised construct A23 | 110 | |
| 8C9 2H6 VH humanised construct A24 | 111 | |
| 8C9 2H6 VH humanised construct A25 | 112 | |
| 8C9 2H6 VH humanised construct A26 | 113 | |
| 8C9 2H6 VH humanised construct A27 | 114 | |
| 8C9 2H6 VH humanised construct A28 | 115 | |
| 8C9 2H6 VL humanised construct M7 | 116 | |
| 8C9 2H6 VL humanised construct M8 | 117 | |
| 8C9 2H6 VL humanised construct M9 | 118 | |
| 8C9 2H6 VL humanised construct M10 | 119 | |
| 8C9 2H6 VL humanised construct M11 | 120 | |
| 8C9 2H6 VL humanised construct M12 | 121 | |
| 8C9 2H6 VL humanised construct M13 | 122 | |
| 8C9 2H6 VL humanised construct M14 | 123 | |

### Sequences

SEQ ID NO: 1
SYGIT
SEQ ID NO: 2
ENYPRSGNTYYNEKFKG
SEQ ID NO: 3
CEFISTVVAPYYYALDY
SEQ ID NO: 4
SEFISTVVAPYYYALDY
SEQ ID NO: 5
KASKKVTIFGSISALH
SEQ ID NO: 6
NGAKLES
SEQ ID NO: 7
LQNKEVPYT
SEQ ID NO: 8
QVQLQQSGAELARPGTSVKLSCKASGYTFTSYGITWVKQRTGQGLEWIGE
NYPRSGNTYYNEKFKGKATLTADKSSSTAYMELRSLTSEDSAVYFCARCE
FISTVVAPYYYALDYWGQGTSVTVSS
SEQ ID NO: 9
CAGGTTCAGCTGCAGCAGTCTGGAGCTGAGCTGGCGAGGCCTGGGACTTC
AGTGAAGCTGTCCTGCAAGGCTTCTGGCTACACCTTCACAAGCTATGGTA
TAACCTGGGTGAAGCAGAGAACTGGACAGGGCCTTGAGTGGATTGGAGAG
AATTATCCTAGAAGTGGTAATACTTACTACAATGAGAAATTCAAGGGCAA
GGCCACACTGACTGCAGACAAATCCTCCAGCACAGCGTACATGGAGCTCC
GCAGCCTGACATCTGAGGACTCTGCGGTCTATTTCTGTGCAAGATGCGAA
TTTATTAGTACGGTAGTAGCTCCCTATTACTATGCTCTGGACTACTGGGG
TCAAGGAACCTCAGTCACCGTCTCCTCA
SEQ ID NO: 10
DIVLTQSPASLAVSLGQKATISCKASKKVTIFGSISALHWYQQKPGQPPK
LIYNGAKLESGVSARFSDSGSQNRSPFGNQLSFTLTIDPVEADDAATYYC
LQNKEVPYTFGGGTKLEIK
SEQ ID NO: 11
GACATTGTACTAACCCAATCTCCAGCATCTTTGGCTGTGTCTCTAGGGCA
GAAGGCCACCATCTCCTGCAAGGCCAGCAAAAAAGTCACTATATTTGGCT
CTATAAGTGCTCTGCACTGGTACCAACAGAAACCAGGACAGCCACCCAAA
CTCATCTATAATGGAGCCAAACTAGAATCTGGGGTCAGTGCCAGGTTCAG
TGACAGTGGGTCTCAGAACCGCTCACCATTTGGAAATCAGCTCAGCTTCA
CCCTCACCATTGATCCTGTGGAGGCTGATGATGCAGCAACCTATTACTGT
CTGCAAAATAAAGAGGTTCCGTACACGTTCGGAGGGGGGACCAAGCTGGA
AATAAAA
SEQ ID NO: 12
QVQLQQSGAELARPGTSVKLSCKASGYTFTSYGITWVKQRTGQGLEWIGE
NYPRSGNTYYNEKFKGKATLTADKSSSTAYMELRSLTSEDSAVYFCARCE
FISTVVAPYYYALDYWGQGTSLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGK
SEQ ID NO: 13
CAGGTTCAGCTGCAGCAGTCTGGAGCTGAGCTGGCGAGGCCTGGGACTTC
AGTGAAGCTGTCCTGCAAGGCTTCTGGCTACACCTTCACAAGCTATGGTA
TAACCTGGGTGAAGCAGAGAACTGGACAGGGCCTTGAGTGGATTGGAGAG
AATTATCCTAGAAGTGGTAATACTTACTACAATGAGAAATTCAAGGGCAA
GGCCACACTGACTGCAGACAAATCCTCCAGCACAGCGTACATGGAGCTCC
GCAGCCTGACATCTGAGGACTCTGCGGTCTATTTCTGTGCAAGATGCGAA
TTTATTAGTACGGTAGTAGCTCCCTATTACTATGCTCTGGACTACTGGGG
TCAAGGAACCTCACTAGTGACCGTGTCCAGCGCCAGCACCAAGGGCCCCA
GCGTGTTCCCCCTGGCCCCCAGCAGCAAGAGCACCAGCGGCGGCACAGCC
GCCCTGGGCTGCCTGGTGAAGGACTACTTCCCCGAACCGGTGACCGTGTC
CTGGAACAGCGGAGCCCTGACCAGCGGCGTGCACACCTTCCCCGCCGTGC
TGCAGAGCAGCGGCCTGTACAGCCTGAGCAGCGTGGTGACCGTGCCCAGC
AGCAGCCTGGGCACCCAGACCTACATCTGTAACGTGAACCACAAGCCCAG
CAACACCAAGGTGGACAAGAAGGTGGAGCCCAAGAGCTGTGACAAGACCC
ACACCTGCCCCCCCTGCCCTGCCCCCGAGCTGCTGGGAGGCCCCAGCGTG
TTCCTGTTCCCCCCCAAGCCTAAGGACACCCTGATGATCAGCAGAACCCC
CGAGGTGACCTGTGTGGTGGTGGATGTGAGCCACGAGGACCCTGAGGTGA
AGTTCAACTGGTACGTGGACGGCGTGGAGGTGCACAATGCCAAGACCAAG
CCCAGGGAGGAGCAGTACAACAGCACCTACCGGGTGGTGTCCGTGCTGAC
CGTGCTGCACCAGGATTGGCTGAACGGCAAGGAGTACAAGTGTAAGGTGT
CCAACAAGGCCCTGCCTGCCCCTATCGAGAAAACCATCAGCAAGGCCAAG
GGCCAGCCCAGAGAGCCCCAGGTGTACACCCTGCCCCCTAGCAGAGATGA
GCTGACCAAGAACCAGGTGTCCCTGACCTGCCTGGTGAAGGGCTTCTACC
CCAGCGACATCGCCGTGGAGTGGGAGAGCAACGGCCAGCCCGAGAACAAC
TACAAGACCACCCCCCCTGTGCTGGACAGCGATGGCAGCTTCTTCCTGTA
CAGCAAGCTGACCGTGGACAAGAGCAGATGGCAGCAGGGCAACGTGTTCA
GCTGCTCCGTGATGCACGAGGCCCTGCACAATCACTACACCCAGAAGAGC
CTGAGCCTGTCCCCTGGCAAG
SEQ ID NO: 14
DIVLTQSPASLAVSLGQKATISCKASKKVTIFGSISALHWYQQKPGQPPK
LIYNGAKLESGVSARFSDSGSQNRSPFGNQLSFTLTIDPVEADDAATYYC
LQNKEVPYTFGGGTKLEIKRTVAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGEC
SEQ ID NO: 15
GACATTGTACTAACCCAATCTCCAGCATCTTTGGCTGTGTCTCTAGGGCA
GAAGGCCACCATCTCCTGCAAGGCCAGCAAAAAAGTCACTATATTTGGCT
CTATAAGTGCTCTGCACTGGTACCAACAGAAACCAGGACAGCCACCCAAA
CTCATCTATAATGGAGCCAAACTAGAATCTGGGGTCAGTGCCAGGTTCAG
TGACAGTGGGTCTCAGAACCGCTCACCATTTGGAAATCAGCTCAGCTTCA
CCCTCACCATTGATCCTGTGGAGGCTGATGATGCAGCAACCTATTACTGT
CTGCAAAATAAAGAGGTTCCGTACACGTTCGGAGGGGGGACCAAGCTGGA
AATAAAACGTACGGTGGCCGCCCCCAGCGTGTTCATCTTCCCCCCCAGCG
ATGAGCAGCTGAAGAGCGGCACCGCCAGCGTGGTGTGTCTGCTGAACAAC
TTCTACCCCCGGGAGGCCAAGGTGCAGTGGAAGGTGGACAATGCCCTGCA
GAGCGGCAACAGCCAGGAGAGCGTGACCGAGCAGGACAGCAAGGACTCCA
CCTACAGCCTGAGCAGCACCCTGACCCTGAGCAAGGCCGACTACGAGAAG
CACAAGGTGTACGCCTGTGAGGTGACCCACCAGGGCCTGTCCAGCCCCGT
GACCAAGAGCTTCAACCGGGGCGAGTGC
SEQ ID NO: 16
QVQLVQSGAEVKKPGSSVKVSCKASGYTFTSYGITWVRQAPGQGLEWMGE
NYPRSGNTYYNEKFKGRVTITADKSTSTAYMELSSLRSEDTAVYYCARSE
FISTVVAPYYYALDYWGQGTLVTVSS
SEQ ID NO: 17
CAGGTGCAGCTGGTGCAGAGCGGCGCCGAAGTGAAGAAGCCCGGCTCCAG
CGTGAAGGTGAGCTGCAAAGCCTCAGGCTACACCTTCACCAGCTACGGCA
TCACTTGGGTGAGGCAGGCCCCCGGCCAGGGACTGGAGTGGATGGGAGAG
AACTACCCCAGGAGCGGCAACACCTACTACAACGAGAAGTTCAAGGGCAG
GGTGACCATCACCGCCGACAAGAGCACCAGCACCGCCTACATGGAGCTGA
GCAGCCTGAGGAGCGAGGACACCGCTGTGTACTACTGCGCCAGGAGCGAG
TTCATCAGCACCGTCGTGGCCCCCTACTACTACGCCCTCGACTATTGGGG
CCAGGGCACACTAGTGACCGTGTCCAGC
SEQ ID NO: 18
DIVMTQSPDSLAVSLGERATINCKASKKVTIFGSISALHWYQQKPGQPPK
LIYNGAKLESGVPDRFSGSGSGTDFTLTISSLQAEDVAVYYCLQNKEVPY
TFGGGTKVEIK
SEQ ID NO: 19
GACATCGTGATGACCCAGAGCCCCGATAGCCTCGCTGTGAGCCTGGGCGA
GAGGGCCACCATCAACTGCAAGGCCAGCAAGAAGGTCACCATCTTCGGCA
GCATCTCCGCCCTGCACTGGTACCAGCAGAAGCCCGGACAGCCCCCCAAG
CTGATCTACAACGGCGCCAAGCTGGAGAGCGGCGTGCCCGACAGGTTTAG
CGGCAGCGGCAGCGGCACAGACTTCACCCTGACCATTAGCAGCCTGCAGG
CCGAAGACGTGGCCGTGTACTACTGCCTGCAGAACAAGGAGGTGCCCTAC
ACCTTCGGCGGGGGCACCAAAGTGGAGATCAAG
SEQ ID NO: 20
DIVMTQSPDSLAVSLGERATINCKASKKVTIFGSISALHWYQQKPGQPPK
LIYNGAKLESGVSDRFSDSGSQNRSPFGNQLSFTLTISSLQAEDVAVYYC
LQNKEVPYTFGGGTKVEIK
SEQ ID NO: 21
GACATCGTGATGACTCAGTCTCCCGACAGCCTGGCCGTGAGCCTGGGCGA
GAGGGCCACCATCAACTGCAAGGCCAGCAAGAAGGTGACCATCTTCGGGA
GCATCTCCGCCCTGCACTGGTATCAGCAGAAACCCGGACAGCCCCCCAAG
CTGATCTACAACGGCGCCAAGCTGGAAAGCGGCGTGAGCGACAGGTTCAG
CGATAGCGGCAGCCAGAACAGGAGCCCTTTCGGCAACCAGCTGAGCTTCA
CCCTGACCATCAGCAGCCTCCAGGCCGAGGACGTCGCAGTGTACTACTGC
CTGCAGAACAAGGAGGTGCCCTACACCTTTGGCGGCGGCACCAAGGTGGA
GATTAAG
SEQ ID NO: 22
DIVMTQTPLSLSVTPGQPASISCKASKKVTIFGSISALHWYLQKPGQPPQ
LIYNGAKLESGVSDRFSDSGSQNRSPFGNQLSFTLKISRVEAEDVGVYYC
LQNKEVPYTFGGGTKVEIK
SEQ ID NO: 23
GATATCGTGATGACCCAGACCCCCCTGAGCCTGAGCGTGACTCCAGGCCA
GCCCGCCAGCATCAGCTGCAAGGCCAGCAAGAAGGTGACCATCTTCGGCA
GCATTAGCGCCCTCCACTGGTACCTGCAGAAACCCGGGCAGCCCCCCCAG
CTGATCTATAACGGCGCTAAGCTGGAGAGCGGCGTGTCCGACAGGTTCAG
CGACTCTGGAAGCCAGAACAGGAGCCCCTTCGGCAACCAGCTGAGCTTCA
CCCTGAAGATCAGCAGGGTGGAAGCCGAGGACGTGGGCGTGTACTACTGC
CTGCAGAACAAGGAGGTGCCCTACACCTTCGGAGGCGGCACCAAGGTCGA
GATCAAG
SEQ ID NO: 24
DIVMTQTPLSLSVTPGQPASISCKASKKVTIFGSISALHWYLQKPGQPPQ
LIYNGAKLESGVSDRFSDSGSGTDFTLKISRVEAEDVGVYYCLQNKEVPY
TFGGGTKVEIK
SEQ ID NO: 25
GACATCGTGATGACCCAGACTCCCCTGTCCCTGAGCGTGACCCCCGGACA
GCCCGCCAGCATCAGCTGCAAGGCCAGCAAGAAGGTGACCATCTTCGGCA
GCATCAGCGCCCTGCACTGGTACCTCCAGAAGCCCGGGCAGCCCCCACAG
CTGATCTACAACGGCGCCAAGCTGGAGAGCGGCGTGAGCGACAGGTTCTC
TGATAGCGGCAGCGGCACCGACTTCACCCTGAAGATTAGCAGGGTGGAGG
CCGAGGACGTGGGCGTGTACTACTGCCTGCAGAACAAGGAGGTGCCCTAC
ACCTTCGGCGGCGGCACCAAAGTCGAGATCAAG
SEQ ID NO: 26
QVQLVQSGAEVKKPGSSVKVSCKASGYTFTSYGITWVRQAPGQGLEWMGE
NYPRSGNTYYNEKFKGRVTITADKSTSTAYMELSSLRSEDTAVYYCARSE
FISTVVAPYYYALDYWGQGTLVTVSSASTKGPSVFPLAPSSKSTSGGTAA
LGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK
SEQ ID NO: 27
CAGGTGCAGCTGGTGCAGAGCGGCGCCGAAGTGAAGAAGCCCGGCTCCAG
CGTGAAGGTGAGCTGCAAAGCCTCAGGCTACACCTTCACCAGCTACGGCA
TCACTTGGGTGAGGCAGGCCCCCGGCCAGGGACTGGAGTGGATGGGAGAG
AACTACCCCAGGAGCGGCAACACCTACTACAACGAGAAGTTCAAGGGCAG
GGTGACCATCACCGCCGACAAGAGCACCAGCACCGCCTACATGGAGCTGA
GCAGCCTGAGGAGCGAGGACACCGCTGTGTACTACTGCGCCAGGAGCGAG
TTCATCAGCACCGTCGTGGCCCCCTACTACTACGCCCTCGACTATTGGGG
CCAGGGCACACTAGTGACCGTGTCCAGCGCCAGCACCAAGGGCCCCAGCG
TGTTCCCCCTGGCCCCCAGCAGCAAGAGCACCAGCGGCGGCACAGCCGCC
CTGGGCTGCCTGGTGAAGGACTACTTCCCCGAACCGGTGACCGTGTCCTG
GAACAGCGGAGCCCTGACCAGCGGCGTGCACACCTTCCCCGCCGTGCTGC
AGAGCAGCGGCCTGTACAGCCTGAGCAGCGTGGTGACCGTGCCCAGCAGC
AGCCTGGGCACCCAGACCTACATCTGTAACGTGAACCACAAGCCCAGCAA
CACCAAGGTGGACAAGAAGGTGGAGCCCAAGAGCTGTGACAAGACCCACA
CCTGCCCCCCCTGCCCTGCCCCCGAGCTGCTGGGAGGCCCCAGCGTGTTC
CTGTTCCCCCCCAAGCCTAAGGACACCCTGATGATCAGCAGAACCCCCGA
GGTGACCTGTGTGGTGGTGGATGTGAGCCACGAGGACCCTGAGGTGAAGT
TCAACTGGTACGTGGACGGCGTGGAGGTGCACAATGCCAAGACCAAGCCC
AGGGAGGAGCAGTACAACAGCACCTACCGGGTGGTGTCCGTGCTGACCGT
GCTGCACCAGGATTGGCTGAACGGCAAGGAGTACAAGTGTAAGGTGTCCA
ACAAGGCCCTGCCTGCCCCTATCGAGAAAACCATCAGCAAGGCCAAGGGC
CAGCCCAGAGAGCCCCAGGTGTACACCCTGCCCCCTAGCAGAGATGAGCT
GACCAAGAACCAGGTGTCCCTGACCTGCCTGGTGAAGGGCTTCTACCCCA
GCGACATCGCCGTGGAGTGGGAGAGCAACGGCCAGCCCGAGAACAACTAC
AAGACCACCCCCCCTGTGCTGGACAGCGATGGCAGCTTCTTCCTGTACAG
CAAGCTGACCGTGGACAAGAGCAGATGGCAGCAGGGCAACGTGTTCAGCT
GCTCCGTGATGCACGAGGCCCTGCACAATCACTACACCCAGAAGAGCCTG
AGCCTGTCCCCTGGCAAG
SEQ ID NO: 28
DIVMTQSPDSLAVSLGERATINCKASKKVTIFGSISALHWYQQKPGQPPK
LIYNGAKLESGVPDRFSGSGSGTDFTLTISSLQAEDVAVYYCLQNKEVPY
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
SEQ ID NO: 29
GACATCGTGATGACCCAGAGCCCCGATAGCCTCGCTGTGAGCCTGGGCGA
GAGGGCCACCATCAACTGCAAGGCCAGCAAGAAGGTCACCATCTTCGGCA
GCATCTCCGCCCTGCACTGGTACCAGCAGAAGCCCGGACAGCCCCCCAAG
CTGATCTACAACGGCGCCAAGCTGGAGAGCGGCGTGCCCGACAGGTTTAG
CGGCAGCGGCAGCGGCACAGACTTCACCCTGACCATTAGCAGCCTGCAGG
CCGAAGACGTGGCCGTGTACTACTGCCTGCAGAACAAGGAGGTGCCCTAC
ACCTTCGGCGGGGGCACCAAAGTGGAGATCAAGCGTACGGTGGCCGCCCC
CAGCGTGTTCATCTTCCCCCCCAGCGATGAGCAGCTGAAGAGCGGCACCG
CCAGCGTGGTGTGTCTGCTGAACAACTTCTACCCCCGGGAGGCCAAGGTG
CAGTGGAAGGTGGACAATGCCCTGCAGAGCGGCAACAGCCAGGAGAGCGT
GACCGAGCAGGACAGCAAGGACTCCACCTACAGCCTGAGCAGCACCCTGA
CCCTGAGCAAGGCCGACTACGAGAAGCACAAGGTGTACGCCTGTGAGGTG
ACCCACCAGGGCCTGTCCAGCCCCGTGACCAAGAGCTTCAACCGGGGCGA
GTGC
SEQ ID NO: 30
DIVMTQSPDSLAVSLGERATINCKASKKVTIFGSISALHWYQQKPGQPPK
LIYNGAKLESGVSDRFSDSGSQNRSPFGNQLSFTLTISSLQAEDVAVYYC
LQNKEVPYTFGGGTKVEIKRTVAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGEC
SEQ ID NO: 31
GACATCGTGATGACTCAGTCTCCCGACAGCCTGGCCGTGAGCCTGGGCGA
GAGGGCCACCATCAACTGCAAGGCCAGCAAGAAGGTGACCATCTTCGGGA
GCATCTCCGCCCTGCACTGGTATCAGCAGAAACCCGGACAGCCCCCCAAG
CTGATCTACAACGGCGCCAAGCTGGAAAGCGGCGTGAGCGACAGGTTCAG
CGATAGCGGCAGCCAGAACAGGAGCCCTTTCGGCAACCAGCTGAGCTTCA
CCCTGACCATCAGCAGCCTCCAGGCCGAGGACGTCGCAGTGTACTACTGC
CTGCAGAACAAGGAGGTGCCCTACACCTTTGGCGGCGGCACCAAGGTGGA
GATTAAGCGTACGGTGGCCGCCCCCAGCGTGTTCATCTTCCCCCCCAGCG
ATGAGCAGCTGAAGAGCGGCACCGCCAGCGTGGTGTGTCTGCTGAACAAC
TTCTACCCCCGGGAGGCCAAGGTGCAGTGGAAGGTGGACAATGCCCTGCA
GAGCGGCAACAGCCAGGAGAGCGTGACCGAGCAGGACAGCAAGGACTCCA
CCTACAGCCTGAGCAGCACCCTGACCCTGAGCAAGGCCGACTACGAGAAG
CACAAGGTGTACGCCTGTGAGGTGACCCACCAGGGCCTGTCCAGCCCCGT
GACCAAGAGCTTCAACCGGGGCGAGTGC
SEQ ID NO: 32
DIVMTQTPLSLSVTPGQPASISCKASKKVTIFGSISALHWYLQKPGQPPQ
LIYNGAKLESGVSDRFSDSGSQNRSPFGNQLSFTLKISRVEAEDVGVYYC
LQNKEVPYTFGGGTKVEIKRTVAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGEC
SEQ ID NO: 33
GATATCGTGATGACCCAGACCCCCCTGAGCCTGAGCGTGACTCCAGGCCA
GCCCGCCAGCATCAGCTGCAAGGCCAGCAAGAAGGTGACCATCTTCGGCA
GCATTAGCGCCCTCCACTGGTACCTGCAGAAACCCGGGCAGCCCCCCCAG
CTGATCTATAACGGCGCTAAGCTGGAGAGCGGCGTGTCCGACAGGTTCAG
CGACTCTGGAAGCCAGAACAGGAGCCCCTTCGGCAACCAGCTGAGCTTCA
CCCTGAAGATCAGCAGGGTGGAAGCCGAGGACGTGGGCGTGTACTACTGC
CTGCAGAACAAGGAGGTGCCCTACACCTTCGGAGGCGGCACCAAGGTCGA
GATCAAGCGTACGGTGGCCGCCCCCAGCGTGTTCATCTTCCCCCCCAGCG
ATGAGCAGCTGAAGAGCGGCACCGCCAGCGTGGTGTGTCTGCTGAACAAC
TTCTACCCCCGGGAGGCCAAGGTGCAGTGGAAGGTGGACAATGCCCTGCA
GAGCGGCAACAGCCAGGAGAGCGTGACCGAGCAGGACAGCAAGGACTCCA
CCTACAGCCTGAGCAGCACCCTGACCCTGAGCAAGGCCGACTACGAGAAG
CACAAGGTGTACGCCTGTGAGGTGACCCACCAGGGCCTGTCCAGCCCCGT
GACCAAGAGCTTCAACCGGGGCGAGTGC
SEQ ID NO: 34
DIVMTQTPLSLSVTPGQPASISCKASKKVTIFGSISALHWYLQKPGQPPQ
LIYNGAKLESGVSDRFSDSGSGTDFTLKISRVEAEDVGVYYCLQNKEVPY
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
SEQ ID NO: 35
GACATCGTGATGACCCAGACTCCCCTGTCCCTGAGCGTGACCCCCGGACA
GCCCGCCAGCATCAGCTGCAAGGCCAGCAAGAAGGTGACCATCTTCGGCA
GCATCAGCGCCCTGCACTGGTACCTCCAGAAGCCCGGGCAGCCCCCACAG
CTGATCTACAACGGCGCCAAGCTGGAGAGCGGCGTGAGCGACAGGTTCTC
TGATAGCGGCAGCGGCACCGACTTCACCCTGAAGATTAGCAGGGTGGAGG
CCGAGGACGTGGGCGTGTACTACTGCCTGCAGAACAAGGAGGTGCCCTAC
ACCTTCGGCGGCGGCACCAAAGTCGAGATCAAGCGTACGGTGGCCGCCCC
CAGCGTGTTCATCTTCCCCCCCAGCGATGAGCAGCTGAAGAGCGGCACCG
CCAGCGTGGTGTGTCTGCTGAACAACTTCTACCCCCGGGAGGCCAAGGTG
CAGTGGAAGGTGGACAATGCCCTGCAGAGCGGCAACAGCCAGGAGAGCGT
GACCGAGCAGGACAGCAAGGACTCCACCTACAGCCTGAGCAGCACCCTGA
CCCTGAGCAAGGCCGACTACGAGAAGCACAAGGTGTACGCCTGTGAGGTG
ACCCACCAGGGCCTGTCCAGCCCCGTGACCAAGAGCTTCAACCGGGGCGA
GTGC
SEQ ID NO: 36
MGWSCIILFLVATATGVHS
SEQ ID NO: 37
MLGSRAVMLLLLLPWTAQGRAVPGGSSPAWTQCQQLSQKLCTLAWSAHPL
VGHMDLREEGDEETTNDVPHIQCGDGCDPQGLRDNSQFCLQRIHQGLIFY
EKLLGSDIFTGEPSLLPDSPVGQLHASLLGLSQLLQPEGHHWETQQIPSL
SPSQPWQRLLLRFKILRSLQAFVAVAARVFAHGAATLSP
SEQ ID NO: 38
ATGCTGGGGAGCAGAGCTGTAATGCTGCTGTTGCTGCT
GCCCTGGACAGCTCAGGGCAGAGCTGTGCCTGGGGGCAGCAGCCCTGCCT
GGACTCAGTGCCAGCAGCTTTCACAGAAGCTCTGCACACTGGCCTGGAGT
GCACATCCACTAGTGGGACACATGGATCTAAGAGAAGAGGGAGATGAAGA
GACTACAAATGATGTTCCCCATATCCAGTGTGGAGATGGCTGTGACCCCC
AAGGACTCAGGGACAACAGTCAGTTCTGCTTGCAAAGGATCCACCAGGGT
CTGATTTTTTATGAGAAGCTGCTAGGATCGGATATTTTCACAGGGGAGCC
TTCTCTGCTCCCTGATAGCCCTGTGGGCCAGCTTCATGCCTCCCTACTGG
GCCTCAGCCAACTCCTGCAGCCTGAGGGTCACCACTGGGAGACTCAGCAG
ATTCCAAGCCTCAGTCCCAGCCAGCCATGGCAGCGTCTCCTTCTCCGCTT
CAAAATCCTTCGCAGCCTCCAGGCCTTTGTGGCTGTAGCCGCCCGGGTCT
TTGCCCATGGAGCAGCAACCCTGAGTCCC
SEQ ID NO: 39
MCHQQLVISWFSLVFLASPLVAIWELKKDVYVVELDWYPDAPGEMVVLTC
DTPEEDGITWTLDQSSEVLGSGKTLTIQVKEFGDAGQYTCHKGGEVLSHS
LLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACP
AAEESLPIEVMVDAVHKLKYENYTSSFFIRDIIKPDPPKNLQLKPLKNSR
QVEVSWEYPDTWSTPHSYFSLTFCVQVQGKSKREKKDRVFTDKTSATVIC
RKNASISVRAQDRYYSSSWSEWASVPCS
SEQ ID NO: 40
ATGTGTCAC
CAGCAGTTGGTCATCTCTTGGTTTTCCCTGGTTTTTCTGGCATCTCCCCT
CGTGGCCATATGGGAACTGAAGAAAGATGTTTATGTCGTAGAATTGGATT
GGTATCCGGATGCCCCTGGAGAAATGGTGGTCCTCACCTGTGACACCCCT
GAAGAAGATGGTATCACCTGGACCTTGGACCAGAGCAGTGAGGTCTTAGG
CTCTGGCAAAACCCTGACCATCCAAGTCAAAGAGTTTGGAGATGCTGGCC
AGTACACCTGTCACAAAGGAGGCGAGGTTCTAAGCCATTCGCTCCTGCTG
CTTCACAAAAAGGAAGATGGAATTTGGTCCACTGATATTTTAAAGGACCA
GAAAGAACCCAAAAATAAGACCTTTCTAAGATGCGAGGCCAAGAATTATT
CTGGACGTTTCACCTGCTGGTGGCTGACGACAATCAGTACTGATTTGACA
TTCAGTGTCAAAAGCAGCAGAGGCTCTTCTGACCCCCAAGGGGTGACGTG
CGGAGCTGCTACACTCTCTGCAGAGAGAGTCAGAGGGGACAACAAGGAGT
ATGAGTACTCAGTGGAGTGCCAGGAGGACAGTGCCTGCCCAGCTGCTGAG
GAGAGTCTGCCCATTGAGGTCATGGTGGATGCCGTTCACAAGCTCAAGTA
TGAAAACTACACCAGCAGCTTCTTCATCAGGGACATCATCAAACCTGACC
CACCCAAGAACTTGCAGCTGAAGCCATTAAAGAATTCTCGGCAGGTGGAG
GTCAGCTGGGAGTACCCTGACACCTGGAGTACTCCACATTCCTACTTCTC
CCTGACATTCTGCGTTCAGGTCCAGGGCAAGAGCAAGAGAGAAAAGAAAG
ATAGAGTCTTCACGGACAAGACCTCAGCCACGGTCATCTGCCGCAAAAAT
GCCAGCATTAGCGTGCGGGCCCAGGACCGCTACTATAGCTCATCTTGGAG
CGAATGGGCATCTGTGCCCTGCAGT
SEQ ID NO: 41
MWPPGSASQPPPSPAAATGLHPAARPVSLQCRLSMCPARSLLLVATLVLL
DHLSLARNLPVATPDPGMFPCLHHSQNLLRAVSNMLQKARQTLEFYPCTS
EEIDHEDITKDKTSTVEACLPLELTKNESCLNSRETSFITNGSCLASRKT
SFMMALCLSSIYEDLKMYQVEFKTMNAKLLMDPKRQIFLDQNMLAVIDEL
MQALNFNSETVPQKSSLEEPDFYKTKIKLCILLHAFRIRAVTIDRVMSYL
NAS
SEQ ID NO: 42
ATGTGGCCCCCTGGGTCAGCCTCCCAGCCACCGCCCTCAC
CTGCCGCGGCCACAGGTCTGCATCCAGCGGCTCGCCCTGTGTCCCTGCAG
TGCCGGCTCAGCATGTGTCCAGCGCGCAGCCTCCTCCTTGTGGCTACCCT
GGTCCTCCTGGACCACCTCAGTTTGGCCAGAAACCTCCCCGTGGCCACTC
CAGACCCAGGAATGTTCCCATGCCTTCACCACTCCCAAAACCTGCTGAGG
GCCGTCAGCAACATGCTCCAGAAGGCCAGACAAACTCTAGAATTTTACCC
TTGCACTTCTGAAGAGATTGATCATGAAGATATCACAAAAGATAAAACCA
GCACAGTGGAGGCCTGTTTACCATTGGAATTAACCAAGAATGAGAGTTGC
CTAAATTCCAGAGAGACCTCTTTCATAACTAATGGGAGTTGCCTGGCCTC
CAGAAAGACCTCTTTTATGATGGCCCTGTGCCTTAGTAGTATTTATGAAG
ACTTGAAGATGTACCAGGTGGAGTTCAAGACCATGAATGCAAAGCTTCTG
ATGGATCCTAAGAGGCAGATCTTTCTAGATCAAAACATGCTGGCAGTTAT
TGATGAGCTGATGCAGGCCCTGAATTTCAACAGTGAGACTGTGCCACAAA
AATCCTCCCTTGAAGAACCGGATTTTTATAAAACTAAAATCAAGCTCTGC
ATACTTCTTCATGCTTTCAGAATTCGGGCAGTGACTATTGATAGAGTGAT
GAGCTATCTGAATGCTTCC
SEQ ID NO: 43
MLGSRAVMLLLLLSWTAQGRAVPGGSSPAWAQCQQLSQKLCTLAWSAHPL
VGHMDLREEGDEETTNDVPHIQCGDGCDPQGLRDNSQFCLQRIRQGLIFY
EKLLGSDIFTGEPSLLPDSPVGQLHASLLGLSQLLQPEGHHWETQQIPSP
SPSQPWQRLLLRFKILRSLQAFVAVAARVFAHGAATLSP
SEQ ID NO: 44
ATGCTGGGGAGCAGAGCTGTAATGCTGCTGTTGCTGCTGTCCTGGACAGC
TCAGGGCAGGGCTGTGCCTGGGGGCAGCAGCCCTGCCTGGGCTCAGTGCC
AGCAGCTTTCACAGAAGCTCTGCACACTGGCCTGGAGTGCACATCCACTA
GTGGGACACATGGATCTAAGAGAAGAGGGAGATGAAGAGACTACAAATGA
TGTTCCCCATATCCAGTGTGGAGATGGCTGTGACCCCCAAGGACTCAGGG
ACAACAGTCAGTTCTGCTTGCAAAGGATTCGCCAGGGTCTGATTTTTTAC
GAGAAGCTACTGGGATCGGATATTTTCACAGGGGAGCCTTCTCTGCTGCC
TGATAGCCCTGTGGGCCAGCTTCATGCCTCCCTACTGGGCCTCAGCCAAC
TCCTGCAGCCTGAGGGTCACCACTGGGAGACTCAGCAGATTCCAAGCCCC
AGTCCCAGCCAGCCATGGCAGCGCCTCCTTCTCCGCTTCAAAATCCTTCG
CAGCCTCCAGGCCTTTGTGGCTGTAGCTGCCCGGGTCTTTGCCCATGGAG
CAGCAACCCTGAGTCCC
SEQ ID NO: 45
MCHQQLVISWFSLVFLASPLMAIWELKKDVYVVELDWYPDAPGEMVVLTC
DTPEEDGITWTLDQSGEVLGSGKTLTIQVKEFGDAGQYTCHKGGEALSHS
LLLLHKKEDGIWSTDVLKDQKEPKNKTFLRCEAKNYSGRFTCWWLTTIST
DLTFSVKSSRGSSNPQGVTCGAVTLSAERVRGDNKEYEYSVECQEDSACP
AAEERLPIEVMVDAIHKLKYENYTSSFFIRDIIKPDPPKNLQLKPLKNSR
QVEVSWEYPDTWSTPHSYFSLTFCIQVQGKSKREKKDRIFTDKTSATVIC
RKNASFSVQAQDRYYSSSWSEWASVPCS
SEQ ID NO: 46
ATGTGTCACCAGCAGCTGGTCATCTCTTGGTTTTCCCTGGTTTTTCTGGC
ATCTCCCCTCATGGCCATATGGGAACTGAAGAAAGACGTTTATGTTGTAG
AATTGGACTGGTACCCGGATGCCCCTGGAGAAATGGTGGTCCTCACCTGT
GACACCCCTGAAGAAGATGGTATCACCTGGACCTTGGACCAGAGTGGTGA
GGTCTTAGGCTCTGGCAAAACCCTGACCATCCAAGTCAAAGAGTTTGGAG
ATGCTGGCCAGTACACCTGTCACAAAGGAGGCGAGGCTCTAAGCCATTCA
CTCCTGCTGCTTCACAAAAAGGAAGATGGAATTTGGTCCACTGATGTTTT
AAAGGACCAGAAAGAACCCAAAAATAAGACCTTTCTAAGATGCGAGGCCA
AAAATTATTCTGGACGTTTCACCTGCTGGTGGCTGACGACAATCAGTACT
GATCTGACATTCAGTGTCAAAAGCAGCAGAGGCTCTTCTAACCCCCAAGG
GGTGACGTGTGGAGCCGTTACACTCTCTGCAGAGAGGGTCAGAGGGGACA
ATAAGGAGTATGAGTACTCAGTGGAGTGCCAGGAGGACAGTGCCTGCCCA
GCCGCTGAGGAGAGGCTGCCCATTGAGGTCATGGTGGATGCCATTCACAA
GCTCAAGTATGAAAACTACACCAGCAGCTTCTTCATCAGGGACATCATCA
AACCCGACCCACCCAAGAACTTGCAGCTGAAGCCATTAAAGAATTCTCGG
CAGGTGGAGGTCAGCTGGGAGTACCCTGACACCTGGAGTACTCCACATTC
CTACTTCTCCCTGACATTCTGCATCCAGGTCCAGGGCAAGAGCAAGAGAG
AAAAGAAAGATAGAATCTTCACAGACAAGACCTCAGCCACGGTCATCTGC
CGCAAAAATGCCAGCTTTAGCGTGCAGGCCCAGGACCGCTACTATAGCTC
ATCTTGGAGCGAATGGGCATCTGTGCCCTGCAGT
SEQ ID NO: 47
MNQVTIQWDAVIALYILFSWCHGGITNINCSGHIWVEPATIFKMGMNISI
YCQAAIKNCQPRKLHFYKNGIKERFQITRINKTTARLWYKNFLEPHASMY
CTAECPKHFQETLICGKDISSGYPPDIPDEVTCVIYEYSGNMTCTWNAGK
LTYIDTKYVVHVKSLETEEEQQYLTSSYINISTDSLQGGKKYLVWVQAAN
ALGMEESKQLQIHLDDIVIPSAAVISRAETINATVPKTIIYWDSQTTIEK
VSCEMRYKATTNQTWNVKEFDTNFTYVQQSEFYLEPNIKYVFQVRCQETG
KRYWQPWSSLFFHKTPETVPQVTSKAFQHDTWNSGLTVASISTGHLTSDN
RGDIGLLLGMIVFAVMLSILSLIGIFNRSFRTGIKRRILLLIPKWLYEDI
PNMKNSNVVKMLQENSELMNNNSSEQVLYVDPMITEIKEIFIPEHKPTDY
KKENTGPLETRDYPQNSLFDNTTVVYIPDLNTGYKPQISNFLPEGSHLSN
NNEITSLTLKPPVDSLDSGNNPRLQKHPNFAFSVSSVNSLSNTIFLGELS
LILNQGECSSPDIQNSVEEETTMLLENDSPSETIPEQTLLPDEFVSCLGI
VNEELPSINTYFPQNILESHFNRISLLEK
SEQ ID NO: 48
QVQLVQSGAEVKKPGSSVKVSCKASGYTFTSYGITWVRQAPGQGLEWMGE
NYPRSGNTYYNEKFKGRVTITADKSTSTAYMELSSLRSEDTAVYYCARAE
FISTVVAPYYYALDYWGQGTLVTVSS
SEQ ID NO: 49
CAGGTGCAGCTGGTGCAGAGCGGCGCCGAAGTGAAGAAGCCCGGCTCCAG
CGTGAAGGTGAGCTGCAAAGCCTCAGGCTACACCTTCACCAGCTACGGCA
TCACTTGGGTGAGGCAGGCCCCCGGCCAGGGACTGGAGTGGATGGGAGAG
AACTACCCCAGGAGCGGCAACACCTACTACAACGAGAAGTTCAAGGGCAG
GGTGACCATCACCGCCGACAAGAGCACCAGCACCGCCTACATGGAGCTGA
GCAGCCTGAGGAGCGAGGACACCGCTGTGTACTACTGCGCCAGGGCTGAG
TTCATCAGCACCGTCGTGGCCCCCTACTACTACGCCCTCGACTATTGGGG
CCAGGGCACACTAGTGACCGTGTCCAGC
SEQ ID NO: 50
QVQLVQSGAEVKKPGSSVKVSCKASGYTFTSYGITWVRQAPGQGLEWMGE
NYPRSGNTYYNEKFKGRVTITADKSTSTAYMELSSLRSEDTAVYYCARVE
FISTVVAPYYYALDYWGQGTLVTVSS
SEQ ID NO: 51
CAGGTGCAGCTGGTGCAGAGCGGCGCCGAAGTGAAGAAGCCCGGCTCCAG
CGTGAAGGTGAGCTGCAAAGCCTCAGGCTACACCTTCACCAGCTACGGCA
TCACTTGGGTGAGGCAGGCCCCCGGCCAGGGACTGGAGTGGATGGGAGAG
AACTACCCCAGGAGCGGCAACACCTACTACAACGAGAAGTTCAAGGGCAG
GGTGACCATCACCGCCGACAAGAGCACCAGCACCGCCTACATGGAGCTGA
GCAGCCTGAGGAGCGAGGACACCGCTGTGTACTACTGCGCCAGGGTGGAG
TTCATCAGCACCGTCGTGGCCCCCTACTACTACGCCCTCGACTATTGGGG
CCAGGGCACACTAGTGACCGTGTCCAGC
SEQ ID NO: 52
QVQLVQSGAEVKKPGSSVKVSCKASGYTFTSYGITWVRQAPGQGLEWMGE
DYPRSGNTYYNEKFKGRVTITADKSTSTAYMELSSLRSEDTAVYYCARSE
FISTVVAPYYYALDYWGQGTLVTVSS
SEQ ID NO: 53
CAGGTGCAGCTGGTGCAGAGCGGCGCCGAAGTGAAGAAGCCCGGCTCCAG
CGTGAAGGTGAGCTGCAAAGCCTCAGGCTACACCTTCACCAGCTACGGCA
TCACTTGGGTGAGGCAGGCCCCCGGCCAGGGACTGGAGTGGATGGGAGAG
GACTACCCCAGGAGCGGCAACACCTACTACAACGAGAAGTTCAAGGGCAG
GGTGACCATCACCGCCGACAAGAGCACCAGCACCGCCTACATGGAGCTGA
GCAGCCTGAGGAGCGAGGACACCGCTGTGTACTACTGCGCCAGGAGCGAG
TTCATCAGCACCGTCGTGGCCCCCTACTACTACGCCCTCGACTATTGGGG
CCAGGGCACACTAGTGACCGTGTCCAGC
SEQ ID NO: 54
QVQLVQSGAEVKKPGSSVKVSCKASGYTFASYGITWVRQAPGQGLEWMGE
NYPRSGNTYYNEKFKGRVTITADKSTSTAYMELSSLRSEDTAMYYCARSE
FISTVVAPYYYALDYWGQGTLVTVSS
SEQ ID NO: 55
CAGGTGCAGCTGGTGCAGAGCGGCGCCGAAGTGAAGAAGCCCGGCTCCAG
CGTGAAGGTGAGCTGCAAAGCCTCAGGCTACACCTTCGCCAGCTACGGCA
TCACTTGGGTGAGGCAGGCCCCCGGCCAGGGACTGGAGTGGATGGGAGAG
AACTACCCCAGGAGCGGCAACACCTACTACAACGAGAAGTTCAAGGGCAG
GGTGACCATCACCGCCGACAAGAGCACCAGCACCGCCTACATGGAGCTGA
GCAGCCTGAGGAGCGAGGACACCGCTATGTACTACTGCGCCAGGAGCGAG
TTCATCAGCACCGTCGTGGCCCCCTACTACTACGCCCTCGACTATTGGGG
CCAGGGCACACTAGTGACCGTGTCCAGC
SEQ ID NO: 56
DIVMTQSPDSLAVSLGERATINCKASKKVTIFGSTSALHWYQQKPGQPPK
LIYNGAKLESGVPDRFSGSGSGTDFTLTISSLQAEDVAVYYCLQNKEVPY
TFGGGTKVEIK
SEQ ID NO: 57
GACATCGTGATGACCCAGAGCCCCGATAGCCTCGCTGTGAGCCTGGGCGA
GAGGGCCACCATCAACTGCAAGGCCAGCAAGAAGGTCACCATCTTCGGCA
GCACCTCCGCCCTGCACTGGTACCAGCAGAAGCCCGGACAGCCCCCCAAG
CTGATCTACAACGGCGCCAAGCTGGAGAGCGGCGTGCCCGACAGGTTTAG
CGGCAGCGGCAGCGGCACAGACTTCACCCTGACCATTAGCAGCCTGCAGG
CCGAAGACGTGGCCGTGTACTACTGCCTGCAGAACAAGGAGGTGCCCTAC
ACCTTCGGCGGGGGCACCAAAGTGGAGATCAAG
SEQ ID NO: 58
DIVMTQSPDSLAVSLGERATINCKASKKVTIFGSTSALHWYQQKPGQPPK
LIYNGAKPESGVPDRFSGSGSGTDFTLTISSLQAEDVAVYYCLQNKEVPY
TFGGGTKVEIK
SEQ ID NO: 59
GACATCGTGATGACCCAGAGCCCCGATAGCCTCGCTGTGAGCCTGGGCGA
GAGGGCCACCATCAACTGCAAGGCCAGCAAGAAGGTCACCATCTTCGGCA
GCACCTCCGCCCTGCACTGGTACCAGCAGAAGCCCGGACAGCCCCCCAAG
CTGATCTACAACGGCGCCAAGCCCGAGAGCGGCGTGCCCGACAGGTTTAG
CGGCAGCGGCAGCGGCACAGACTTCACCCTGACCATTAGCAGCCTGCAGG
CCGAAGACGTGGCCGTGTACTACTGCCTGCAGAACAAGGAGGTGCCCTAC
ACCTTCGGCGGGGGCACCAAAGTGGAGATCAAG
SEQ ID NO: 60
QVQLVQSGAEVKKPGSSVKVSCKASGYTFTSYGITWVRQAPGQGLEWMGE
NYPRSGNTYYNEKFKGRVTITADKSTSTAYMELSSLRSEDTAVYYCARAE
FISTVVAPYYYALDYWGQGTLVTVSSASTKGPSVFPLAPSSKSTSGGTAA
LGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK
SEQ ID NO: 61
CAGGTGCAGCTGGTGCAGAGCGGCGCCGAAGTGAAGAAGCCCGGCTCCAG
CGTGAAGGTGAGCTGCAAAGCCTCAGGCTACACCTTCACCAGCTACGGCA
TCACTTGGGTGAGGCAGGCCCCCGGCCAGGGACTGGAGTGGATGGGAGAG
AACTACCCCAGGAGCGGCAACACCTACTACAACGAGAAGTTCAAGGGCAG
GGTGACCATCACCGCCGACAAGAGCACCAGCACCGCCTACATGGAGCTGA
GCAGCCTGAGGAGCGAGGACACCGCTGTGTACTACTGCGCCAGGGCTGAG
TTCATCAGCACCGTCGTGGCCCCCTACTACTACGCCCTCGACTATTGGGG
CCAGGGCACACTAGTGACCGTGTCCAGCGCCAGCACCAAGGGCCCCAGCG
TGTTCCCCCTGGCCCCCAGCAGCAAGAGCACCAGCGGCGGCACAGCCGCC
CTGGGCTGCCTGGTGAAGGACTACTTCCCCGAACCGGTGACCGTGTCCTG
GGAACAGCGGAGCCCTGACCAGCGGCGTGCACACCTTCCCCCCGTGCTGC
AGAGCAGCGGCCTGTACAGCCTGAGCAGCGTGGTGACCGTGCCCAGCAGC
AGCCTGGGCACCCAGACCTACATCTGTAACGTGAACCACAAGCCCAGCAA
CACCAAGGTGGACAAGAAGGTGGAGCCCAAGAGCTGTGACAAGACCCACA
CCTGCCCCCCCTGCCCTGCCCCCGAGCTGCTGGGAGGCCCCAGCGTGTTC
CTGTTCCCCCCCAAGCCTAAGGACACCCTGATGATCAGCAGAACCCCCGA
GGGTGACCTGTGTGGTGGTGGATGTGAGCCACGAGGACCCTAGGTGAAGT
TCAACTGGTACGTGGACGGCGTGGAGGTGCACAATGCCAAGACCAAGCCC
AGGGAGGAGCAGTACAACAGCACCTACCGGGTGGTGTCCGTGCTGACCGT
GCTGCACCAGGATTGGCTGAACGGCAAGGAGTACAAGTGTAAGGTGTCCA
ACAAGGCCCTGCCTGCCCCTATCGAGAAAACCATCAGCAAGGCCAAGGGC
CAGCCCAGAGAGCCCCAGGTGTACACCCTGCCCCCTAGCAGAGATGAGCT
GACCAAGAACCAGGTGTCCCTGACCTGCCTGGTGAAGGGCTTCTACCCCA
GCGACATCGCCGTGGAGTGGGAGAGCAACGGCCAGCCCGAGAACAACTAC
AAGACCACCCCCCCTGTGCTGGACAGCGATGGCAGCTTCTTCCTGTACAG
CAAGCTGACCGTGGACAAGAGCAGATGGCAGCAGGGCAACGTGTTCAGCT
GCTCCGTGATGCACGAGGCCCTGCACAATCACTACACCCAGAAGAGCCTG
AGCCTGTCCCCTGGCAAG
SEQ ID NO: 62
QVQLVQSGAEVKKPGSSVKVSCKASGYTFTSYGITWVRQAPGQGLEWMGE
NYPRSGNTYYNEKFKGRVTITADKSTSTAYMELSSLRSEDTAVYYCARVE
FISTVVAPYYYALDYWGQGTLVTVSSASTKGPSVFPLAPSSKSTSGGTAA
LGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK
SEQ D NO: 63
CAGGTGCAGCTGGTGCAGAGCGGCGCCGAAGTGAAGAAGCCCGGCTCCAG
CGTGAAGGTGAGCTGCAAAGCCTCAGGCTACACCTTCACCAGCTACGGCA
TCACTTGGGTGAGGCAGGCCCCCGGCCAGGGACTGGAGTGGATGGGAGAG
AACTACCCCAGGAGCGGCAACACCTACTACAACGAGAAGTTCAAGGGCAG
GGTGACCATCACCGCCGACAAGAGCACCAGCACCGCCTACATGGAGCTGA
GCAGCCTGAGGAGCGAGGACACCGCTGTGTACTACTGCGCCAGGGTGGAG
TTCATCAGCACCGTCGTGGCCCCCTACTACTACGCCCTCGACTATTGGGG
CCAGGGCACACTAGTGACCGTGTCCAGCGCCAGCACCAAGGGCCCCAGCG
TGTTCCCCCTGGCCCCCAGCAGCAAGAGCACCAGCGGCGGCACAGCCGCC
CTGGGCTGCCTGGTGAAGGACTACTTCCCCGAACCGGTGACCGTGTCCTG
GAACAGCGGAGCCCTGACCAGCGGCGTGCACACCTTCCCCGCCGTGCTGC
AGAGCAGCGGCCTGTACAGCCTGAGCAGCGTGGTGACCGTGCCCAGCAGC
AGCCTGGGCACCCAGACCTACATCTGTAACGTGAACCACAAGCCCAGCAA
CACCAAGGTGGACAAGAAGGTGGAGCCCAAGAGCTGTGACAAGACCCACA
CCTGCCCCCCCTGCCCTGCCCCCGAGCTGCTGGGAGGCCCCAGCGTGTTC
CTGTTCCCCCCCAAGCCTAAGGACACCCTGATGATCAGCAGAACCCCCGA
GGTGACCTGTGTGGTGGTGGATGTGAGCCACGAGGACCCTGAGGTGAAGT
TCAACTGGTACGTGGACGGCGTGGAGGTGCACAATGCCAAGACCAAGCCC
AGGGAGGAGCAGTACAACAGCACCTACCGGGTGGTGTCCGTGCTGACCGT
GCTGCACCAGGATTGGCTGAACGGCAAGGAGTACAAGTGTAAGGTGTCCA
ACAAGGCCCTGCCTGCCCCTATCGAGAAAACCATCAGCAAGGCCAAGGGC
CAGCCCAGAGAGCCCCAGGTGTACACCCTGCCCCCTAGCAGAGATGAGCT
GACCAAGAACCAGGTGTCCCTGACCTGCCTGGTGAAGGGCTTCTACCCCA
GCGACATCGCCGTGGAGTGGGAGAGCAACGGCCAGCCCGAGAACAACTAC
AAGACCACCCCCCCTGTGCTGGACAGCGATGGCAGCTTCTTCCTGTACAG
CAAGCTGACCGTGGACAAGAGCAGATGGCAGCAGGGCAACGTGTTCAGCT
GCTCCGTGATGCACGAGGCCCTGCACAATCACTACACCCAGAAGAGCCTG
AGCCTGTCCCCTGGCAAG
SEQ ID NO: 64
QVQLVQSGAEVKKPGSSVKVSCKASGYTFTSYGITWVRQAPGQGLEWMGE
DYPRSGNTYYNEKFKGRVTITADKSTSTAYMELSSLRSEDTAVYYCARSE
FISTVVAPYYYALDYWGQGTLVTVSSASTKGPSVFPLAPSSKSTSGGTAA
LGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK
SEQ ID NO: 65
CAGGTGCAGCTGGTGCAGAGCGGCGCCGAAGTGAAGAAGCCCGGCTCCAG
CGTGAAGGTGAGCTGCAAAGCCTCAGGCTACACCTTCACCAGCTACGGCA
TCACTTGGGTGAGGCAGGCCCCCGGCCAGGGACTGGAGTGGATGGGAGAG
GACTACCCCAGGAGCGGCAACACCTACTACAACGAGAAGTTCAAGGGCAG
GGTGACCATCACCGCCGACAAGAGCACCAGCACCGCCTACATGGAGCTGA
GCAGCCTGAGGAGCGAGGACACCGCTGTGTACTACTGCGCCAGGAGCGAG
TTCATCAGCACCGTCGTGGCCCCCTACTACTACGCCCTCGACTATTGGGG
CCAGGGCACACTAGTGACCGTGTCCAGCGCCAGCACCAAGGGCCCCAGCG
TGTTCCCCCTGGCCCCCAGCAGCAAGAGCACCAGCGGCGGCACAGCCGCC
CTGGGCTGCCTGGTGAAGGACTACTTCCCCGAACCGGTGACCGTGTCCTG
GAACAGCGGAGCCCTGACCAGCGGCGTGCACACCTTCCCCGCCGTGCTGC
AGAGCAGCGGCCTGTACAGCCTGAGCAGCGTGGTGACCGTGCCCAGCAGC
AGCCTGGGCACCCAGACCTACATCTGTAACGTGAACCACAAGCCCAGCAA
CACCAAGGTGGACAAGAAGGTGGAGCCCAAGAGCTGTGACAAGACCCACA
CCTGCCCCCCCTGCCCTGCCCCCGAGCTGCTGGGAGGCCCCAGCGTGTTC
CTGTTCCCCCCCAAGCCTAAGGACACCCTGATGATCAGCAGAACCCCCGA
GGTGACCTGTGTGGTGGTGGATGTGAGCCACGAGGACCCTGAGGTGAAGT
TCAACTGGTACGTGGACGGCGTGGAGGTGCACAATGCCAAGACCAAGCCC
AGGGAGGAGCAGTACAACAGCACCTACCGGGTGGTGTCCGTGCTGACCGT
GCTGCACCAGGATTGGCTGAACGGCAAGGAGTACAAGTGTAAGGTGTCCA
ACAAGGCCCTGCCTGCCCCTATCGAGAAAACCATCAGCAAGGCCAAGGGC
CAGCCCAGAGAGCCCCAGGTGTACACCCTGCCCCCTAGCAGAGATGAGCT
GACCAAGAACCAGGTGTCCCTGACCTGCCTGGTGAAGGGCTTCTACCCCA
GCGACATCGCCGTGGAGTGGGAGAGCAACGGCCAGCCCGAGAACAACTAC
AAGACCACCCCCCCTGTGCTGGACAGCGATGGCAGCTTCTTCCTGTACAG
CAAGCTGACCGTGGACAAGAGCAGATGGCAGCAGGGCAACGTGTTCAGCT
GCTCCGTGATGCACGAGGCCCTGCACAATCACTACACCCAGAAGAGCCTG
AGCCTGTCCCCTGGCAAG
SEQ ID NO: 66
QVQLVQSGAEVKKPGSSVKVSCKASGYTFASYGITWVRQAPGQGLEWMGE
NYPRSGNTYYNEKFKGRVTITADKSTSTAYMELSSLRSEDTAMYYCARSE
FISTVVAPYYYALDYWGQGTLVTVSSASTKGPSVFPLAPSSKSTSGGTAA
LGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK
SEQ ID NO: 67
CAGGTGCAGCTGGTGCAGAGCGGCGCCGAAGTGAAGAAGCCCGGCTCCAG
CGTGAAGGTGAGCTGCAAAGCCTCAGGCTACACCTTCGCCAGCTACGGCA
TCACTTGGGTGAGGCAGGCCCCCGGCCAGGGACTGGAGTGGATGGGAGAG
AACTACCCCAGGAGCGGCAACACCTACTACAACGAGAAGTTCAAGGGCAG
GGTGACCATCACCGCCGACAAGAGCACCAGCACCGCCTACATGGAGCTGA
GCAGCCTGAGGAGCGAGGACACCGCTATGTACTACTGCGCCAGGAGCGAG
TTCATCAGCACCGTCGTGGCCCCCTACTACTACGCCCTCGACTATTGGGG
CCAGGGCACACTAGTGACCGTGTCCAGCGCCAGCACCAAGGGCCCCAGCG
TGTTCCCCCTGGCCCCCAGCAGCAAGAGCACCAGCGGCGGCACAGCCGCC
CTGGGCTGCCTGGTGAAGGACTACTTCCCCGAACCGGTGACCGTGTCCTG
GAACAGCGGAGCCCTGACCAGCGGCGTGCACACCTTCCCCGCCGTGCTGC
AGAGCAGCGGCCTGTACAGCCTGAGCAGCGTGGTGACCGTGCCCAGCAGC
AGCCTGGGCACCCAGACCTACATCTGTAACGTGAACCACAAGCCCAGCAA
CACCAAGGTGGACAAGAAGGTGGAGCCCAAGAGCTGTGACAAGACCCACA
CCTGCCCCCCCTGCCCTGCCCCCGAGCTGCTGGGAGGCCCCAGCGTGTTC
CTGTTCCCCCCCAAGCCTAAGGACACCCTGATGATCAGCAGAACCCCCGA
GGTGACCTGTGTGGTGGTGGATGTGAGCCACGAGGACCCTGAGGTGAAGT
TCAACTGGTACGTGGACGGCGTGGAGGTGCACAATGCCAAGACCAAGCCC
AGGGAGGAGCAGTACAACAGCACCTACCGGGTGGTGTCCGTGCTGACCGT
GCTGCACCAGGATTGGCTGAACGGCAAGGAGTACAAGTGTAAGGTGTCCA
ACAAGGCCCTGCCTGCCCCTATCGAGAAAACCATCAGCAAGGCCAAGGGC
CAGCCCAGAGAGCCCCAGGTGTACACCCTGCCCCCTAGCAGAGATGAGCT
GACCAAGAACCAGGTGTCCCTGACCTGCCTGGTGAAGGGCTTCTACCCCA
GCGACATCGCCGTGGAGTGGGAGAGCAACGGCCAGCCCGAGAACAACTAC
AAGACCACCCCCCCTGTGCTGGACAGCGATGGCAGCTTCTTCCTGTACAG
CAAGCTGACCGTGGACAAGAGCAGATGGCAGCAGGGCAACGTGTTCAGCT
GCTCCGTGATGCACGAGGCCCTGCACAATCACTACACCCAGAAGAGCCTG
AGCCTGTCCCCTGGCAAG
SEQ ID NO: 68
DIVMTQSPDSLAVSLGERATINCKASKKVTIFGSTSALHWYQQKPGQPPK
LIYNGAKLESGVPDRFSGSGSGTDFTLTISSLQAEDVAVYYCLQNKEVPY
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
SEQ ID NO: 69
GACATCGTGATGACCCAGAGCCCCGATAGCCTCGCTGTGAGCCTGGGCGA
GAGGGCCACCATCAACTGCAAGGCCAGCAAGAAGGTCACCATCTTCGGCA
GCACCTCCGCCCTGCACTGGTACCAGCAGAAGCCCGGACAGCCCCCCAAG
CTGATCTACAACGGCGCCAAGCTGGAGAGCGGCGTGCCCGACAGGTTTAG
CGGCAGCGGCAGCGGCACAGACTTCACCCTGACCATTAGCAGCCTGCAGG
CCGAAGACGTGGCCGTGTACTACTGCCTGCAGAACAAGGAGGTGCCCTAC
ACCTTCGGCGGGGGCACCAAAGTGGAGATCAAGCGTACGGTGGCCGCCCC
CAGCGTGTTCATCTTCCCCCCCAGCGATGAGCAGCTGAAGAGCGGCACCG
CCAGCGTGGTGTGTCTGCTGAACAACTTCTACCCCCGGGAGGCCAAGGTG
CAGTGGAAGGTGGACAATGCCCTGCAGAGCGGCAACAGCCAGGAGAGCGT
GACCGAGCAGGACAGCAAGGACTCCACCTACAGCCTGAGCAGCACCCTGA
CCCTGAGCAAGGCCGACTACGAGAAGCACAAGGTGTACGCCTGTGAGGTG
ACCCACCAGGGCCTGTCCAGCCCCGTGACCAAGAGCTTCAACCGGGGCGA
GTGC
SEQ ID NO: 70
DIVMTQSPDSLAVSLGERATINCKASKKVTIFGSTSALHWYQQKPGQPPK
LIYNGAKPESGVPDRFSGSGSGTDFTLTISSLQAEDVAVYYCLQNKEVPY
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
SEQ ID NO: 71
GACATCGTGATGACCCAGAGCCCCGATAGCCTCGCTGTGAGCCTGGGCGA
GAGGGCCACCATCAACTGCAAGGCCAGCAAGAAGGTCACCATCTTCGGCA
GCACCTCCGCCCTGCACTGGTACCAGCAGAAGCCCGGACAGCCCCCCAAG
CTGATCTACAACGGCGCCAAGCCCGAGAGCGGCGTGCCCGACAGGTTTAG
CGGCAGCGGCAGCGGCACAGACTTCACCCTGACCATTAGCAGCCTGCAGG
CCGAAGACGTGGCCGTGTACTACTGCCTGCAGAACAAGGAGGTGCCCTAC
ACCTTCGGCGGGGGCACCAAAGTGGAGATCAAGCGTACGGTGGCCGCCCC
CAGCGTGTTCATCTTCCCCCCCAGCGATGAGCAGCTGAAGAGCGGCACCG
CCAGCGTGGTGTGTCTGCTGAACAACTTCTACCCCCGGGAGGCCAAGGTG
CAGTGGAAGGTGGACAATGCCCTGCAGAGCGGCAACAGCCAGGAGAGCGT
GACCGAGCAGGACAGCAAGGACTCCACCTACAGCCTGAGCAGCACCCTGA
CCCTGAGCAAGGCCGACTACGAGAAGCACAAGGTGTACGCCTGTGAGGTG
ACCCACCAGGGCCTGTCCAGCCCCGTGACCAAGAGCTTCAACCGGGGCGA
GTGC
SEQ ID NO: 72
EDYPRSGNTYYNEKFKG
SEQ ID NO: 73
AEFISTVVAPYYYALDY
SEQ ID NO: 74
VEFISTVVAPYYYALDY
SEQ ID NO: 75
KASKKVTIFGSTSALH
SEQ ID NO: 76
NGAKPES
SEQ ID NO: 77
DGAKLES
SEQ ID NO: 78
QGAKLES
SEQ ID NO: 79
DGAKPES
SEQ ID NO: 80
QGAKPES
SEQ ID NO: 81
QVQLVQSGAEVKKPGSSVKVSCKASGYTFTSYGITWVRQAPGQGLEWMGE
DYPRSGNTYYNEKFKGRVTITADKSTSTAYMELSSLRSEDTAVYYCARAE
FISTVVAPYYYALDYWGQGTLVTVSS
SEQ ID NO: 82
QVQLVQSGAEVKKPGSSVKVSCKASGYTFTSYGITWVRQAPGQGLEWMGE
DYPRSGNTYYNEKFKGRVTITADKSTSTAYMELSSLRSEDTAVYYCARVE
FISTVVAPYYYALDYWGQGTLVTVSS
SEQ ID NO: 83
QVQLVQSGAEVKKPGSSVKVSCKASGYTFASYGITWVRQAPGQGLEWMGE
NNYPRSGNTYYEKFKGRVTITADKSTSTAYMELSSLRSEDTAMYYCARAE
FISTVVAPYYYALDYWGQGTLVTVSS
SEQ ID NO: 84
QVQLVQSGAEVKKPGSSVKVSCKASGYTFASYGITWVRQAPGQGLEWMGE
NYPRSGNTYYNEKFKGRVTITADKSTSTAYMELSSLRSEDTAMYYCARVE
FISTVVAPYYYALDYWGQGTLVTVSS
SEQ ID NO: 85
QVQLVQSGAEVKKPGSSVKVSCKASGYTFASYGITWVRQAPGQGLEWMGE
NYPRSGNTYYNEKFKGRVTITADKSTSTAYMELSSLRSEDTAVYYCARSE
FISTVVAPYYYALDYWGQGTLVTVSS
SEQ ID NO: 86
QVQLVQSGAEVKKPGSSVKVSCKASGYTFASYGITWVRQAPGQGLEWMGE
NYPRSGNTYYNEKFKGRVTITADKSTSTAYMELSSLRSEDTAVYYCARAE
FISTVVAPYYYALDYWGQGTLVTVSS
SEQ ID NO: 87
QVQLVQSGAEVKKPGSSVKVSCKASGYTFASYGITWVRQAPGQGLEWMGE
NYPRSGNTYYNEKFKGRVTITADKSTSTAYMELSSLRSEDTAVYYCARVE
FISTVVAPYYYALDYWGQGTLVTVSS
SEQ ID NO: 88
QVQLVQSGAEVKKPGSSVKVSCKASGYTFASYGITWVRQAPGQGLEWMGE
DYPRSGNTYYNEKFKGRVTITADKSTSTAYMELSSLRSEDTAVYYCARSE
FISTVVAPYYYALDYWGQGTLVTVSS
SEQ ID NO: 89
QVQLVQSGAEVKKPGSSVKVSCKASGYTFASYGITWVRQAPGQGLEWMGE
DYPRSGNTYYNEKFKGRVTITADKSTSTAYMELSSLRSEDTAVYYCARAE
FISTVVAPYYYALDYWGQGTLVTVSS
SEQ ID NO: 90
QVQLVQSGAEVKKPGSSVKVSCKASGYTFASYGITWVRQAPGQGLEWMGE
DYPRSGNTYYNEKFKGRVTITADKSTSTAYMELSSLRSEDTAVYYCARVE
FISTVVAPYYYALDYWGQGTLVTVSS
SEQ ID NO: 91
RTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
SEQ ID NO: 92
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO: 93
DIVMTQSPDSLAVSLGERATINCKASKKVTIFGSISALHWYQQKPGQPPK
LIYDGAKLESGVPDRFSGSGSGTDFTLTISSLQAEDVAVYYCLQNKEVPY
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
SEQ ID NO: 94
DIVMTQSPDSLAVSLGERATINCKASKKVTIFGSISALHWYQQKPGQPPK
LIYQGAKLESGVPDRFSGSGSGTDFTLTISSLQAEDVAVYYCLQNKEVPY
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
SEQ ID NO: 95
SEFISTVMAPYYYALDY
SEQ ID NO: 96
DIVMTQSPDSLAVSLGERATINCKASKKVTIFGSISALHWYQQKPGQPPK
LIYDGAKLESGVPDRFSGSGSGTDFTLTISSLQAEDVAVYYCLQNKEVPY
TFGGGTKVEIK
SEQ ID NO: 97
DIVMTQSPDSLAVSLGERATINCKASKKVTIFGSISALHWYQQKPGQPPK
LIYQGAKLESGVPDRFSGSGSGTDFTLTISSLQAEDVAVYYCLQNKEVPY
TFGGGTKVEIK
SEQ ID NO: 98
ENYPRSGNIYYNEKFKG
SEQ ID NO: 99
ENYPRSGNTYYNEKFRG
SEQ ID NO: 100
SEFTSTVVAPYYYALDY
SEQ ID NO: 101
KASKKVTIYGSTSALH
SEQ ID NO: 102
NSAKLES
SEQ ID NO: 103
QVQLVQSGAEVKKPGSSVKVSCKASGYTFTSYGITWVRQAPGQGLEWMGE
NYPRSGNTYYNEKFKGRVTITADKSTSTAYMELSSLRSEDTAVYYCARSE
FISTVMAPYYYALDYWGQGTLVTVSS
SEQ ID NO: 104
QVQLVQSGAEVKKPGSSVKVSCKASGYTFTSYGITWVRQAPGQGLEWMGE
DYPRSGNTYYNEKFKGRVTITADKSTSTAYMELSGLRSEDTAVYYCARSE
FISTVVAPYYYALDYWGQGTLVTVSS
SEQ ID NO: 105
QVQLVQSGAEVKKPGSSVRVSCKASGYTFTSYGITWVRQAPGQGLEWMGE
NYPRSGNTYYNEKFKGRVTITADKSTSTAYMELSSLRSEDTAVYYCARSE
FISTVVAPYYYALDYWGQGTLVTVSS
SEQ ID NO: 106
QVQLVQSGAEVKKPGSSVKVSCKASGYTFASYGITWVRQAPGQGLEWMGE
NYPRSGNTYYNEKFKGRVTITADKSTSTAYMELSSLRSEDTAAYYCARSE
FISTVVAPYYYALDYWGQGTLVTVSS
SEQ ID NO: 107
QVQLVQSGAEVKKPGSSVKVSCEASGYTFTSYGITWVRQAPGQGLEWMGE
NYPRSGNTYYNEKFKGRVTITADKSTSTAYMELSSLRSEDTAVYYCARSE
FISTVVAPYYYALDYWGQGTLVTVSS
SEQ ID NO: 108
QVQLVQSGAEVKKPGSSVKVSCKASGYTFTSYGITWVRQAPGQGLEWMGE
NYPRSGNTYYNEKFKGRVTITADKSTNTAYMELSSLRSEDTAVYYCARSE
FISTVVAPYYYALDYWGQGTLVTVSS
SEQ ID NO: 109
QVQLVQSGAEVKKPGSSVKVNCKASGYTFTSYGITWVRQAPGQGLEWMGE
NYPRSGNTYYNEKFKGRVTITADKSTSTAYMELSSLRSEDTAVYYCARSE
FISTVVAPYYYALDYWGQGTLVTVSS
SEQ ID NO: 110
QVQLVQSGAEVKKPGSSVKVSCKASGYTFTSYGITWVRQAPGQGLEWMGE
NYPRSGNTYYNEKFRGRVTITADKSTSTAYMELSSLRSEDTAVYYCARSE
FISTVVAPYYYALDYWGQGTLVTVSS
SEQ ID NO: 111
QVQLVQSGAEVKKPGSSVKVSCKASGYTFTSYGITWVRQAPGQGLEWMGE
NYPRSGNIYYNEKFKGRVTITADKSTSTAYMELSSLRSEDTAVYYCARSE
FISTVVAPYYYALDYWGQGTLVTVSS
SEQ ID NO: 112
QVQLVQSGAEVKKPGSSVKVSCKASGYTFTSYGITWVRQAPGQGLEWMGE
NYPRSGNTYYNEKFKGRVTITADKSTSTAYMELSGLRSEDTAVYYCARSE
FISTVVAPYYYALDYWGQGTLVTVSS
SEQ ID NO: 113
QVQLVQSGAEVKKPGSSVKVSCKASGYTFASYGITWVRQAPGQGLEWMGE
NYPRSGNTYYNEKFRGRVTITADKSTSTAYMELSSLRSEDTAVYYCARSE
FISTVVAPYYYALDYWGQGTLVTVSS
SEQ ID NO: 114
QVQLVQSSAEVKKPGSSVKVSCKASGYTFASYGITWVRQAPGQGLEWMGE
NYPRSGNTYYNEKFKGRVTITADKSTSTAYMELSSLRSEDTAVYYCARSE
FTSTVVAPYYYALDYWGQGTLVTVSS
SEQ ID NO: 115
QVQLVQSGAEVKKPGSSVKVSCKASGYTFASYGITWVRQAPGQGLEWMGE
NYPRSGNTYYNEKFKGRVTITADKSTGTAYMELSSLRSEDTAVYYCARSE
FISTVVAPYYYALDYWGQGTLVTVSS
SEQ ID NO: 116
DIVMTQSPDSLVVSLGERATINCKASKKVTIFGSTSALHWYQQKPGQPPK
LIYNGAKLESGVPDRFSGSGSGTDFTLTISSLQAEDVAVYYCLQNKEVPY
TFGGGTKVEIK
SEQ ID NO: 117
DIVMTQSPDSLAVSLGERATINCKASKKVTIFGSISALHWYQQKPGQPPK
LVYNGAKLESGVPDRFSGSGSGADFTLTISSLQAEDVAVYYCLQNKEVPY
TFGGGTKVEIK
SEQ ID NO: 118
DIVMTQSPDSLAVSLGERATINCKASKKVTIFGSISALHWYQQRPGQPPK
LIYNGAKLESGVPDRFSGSGSGTDFTLTISSLQAEDVAVYYCLQNKEVPY
TFGGGTKVEIK
SEQ ID NO: 119
DIVMTQSPDSLAVSLGERATINCKASKKVTIFGSISALHWYQQKPGQPPK
LIYNGAKLESGVPGRFSGSGSGTDFTLTISSLQAEDVAVYYCLQNKEVPY
TFGGGTKVEIK
SEQ ID NO: 120
DIVMTQSPDSLAVSLGERATINCKASKKVTIFGSISALHWYQQKPGQPPK
LIYNSAKLESGVPDRFSGSGSGTDFTLTISSLQAEDVAVYYCLQNKEVPY
TFGGGTKVEIK
SEQ ID NO: 121
DIVMTQSPDSLAVSLGERATINCKASKKVTIYGSTSALHWYQQKPGQPPK
LIYNGAKPESGVPDRFSGSGSGTDFTLTISSLQAEDVAVYYCLQNKEVPY
TFGGGTKVEIK
SEQ ID NO: 122
DIVMTQSPDSLAVSLGERATISCKASKKVTIFGSTSALHWYQQKPGQPPK
LIYNGAKPESGVPDRFSGSGSGTDFTLTISSLQAEDVAVYYCLQNKEVPY
TFGGGTKVEIK
SEQ ID NO: 123
GIVMTQSPDSLAVSLGERATINCKASKKVTIFGSTSALHWYQQKPGQPPK
LIYNGAKLESGVPDRFSGSGSGTDFTLTISSLQAEDVAVYYCLQNKEVPY
TFGGGTKVEIK

1. An antigen binding protein which binds human IL-23 and which comprises the CDRH30f SEQ ID NO: 3, SEQ ID NO: 4, SEQ ID NO: 73, SEQ ID NO: 74, SEQ ID NO: 95 or SEQ ID NO: 100, or variants thereof which contain 1 or 2 or 3 amino acid substitutions in CDRH3.

2. An antigen binding protein according to claim 1 wherein said antigen binding protein comprises the following CDRs:
CDRH1: SEQ. I.D. NO:1
CDRH2: SEQ. I.D. NO:2
CDRH3: SEQ. I.D. NO:4
CDRL1: SEQ. I.D. NO:5
CDRL2: SEQ. I.D. NO:6 and
CDRL3: SEQ. I.D. NO:7

3. An antigen binding protein which binds the same epitope as the antigen binding protein of claim 1 or 2 and neutralises human IL-23.

4. An antigen binding protein according to claim 1 that neutralises both human IL-23 and cynomolgus IL-23.

5. An antigen binding protein according to claim 1 that neutralises human IL-23 but does not neutralise human IL-12.

6. An antigen binding protein according to claim 1 wherein the antigen binding protein is an antibody.

7. An antibody according to claim 6 wherein the antibody is a humanised or chimeric antibody.

8. An antigen binding protein which competes with the antigen binding protein of claim 1 or 2 and neutralises human IL-23.

9. An antigen binding protein of claim 6 wherein the antibody is of IgG isotype.

10. The antigen binding protein of claim 9 wherein the human antibody constant region is IgG1.

11. An antigen binding protein according to claim 1 comprising a VH domain selected from SEQ ID NO: 16, SEQ ID NO: 48, SEQ ID NO: 50, SEQ ID NO: 52, SEQ ID NO: 54, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114 and SEQ ID NO: 115; and a VL domain selected from SEQ ID NO: 18, SEQ ID NO: 20, SEQ ID NO: 22, SEQ ID NO: 24, SEQ ID NO:56, SEQ ID NO:58, SEQ ID NO:96, SEQ ID NO:116, SEQ ID NO:117, SEQ ID NO:118, SEQ ID NO:119, SEQ ID NO:120, SEQ ID NO:121, SEQ ID NO:122, and SEQ ID NO:123.

12. An antigen binding protein according to claim 1 wherein the antigen binding protein comprises a Fab, Fab′, F(ab′)2, Fv, diabody, triabody, tetrabody, miniantibody, minibody, isolated VH, isolated VL or a dAb.

13. An antibody according to claim 6 comprising a mutated Fc region such that said antibody has reduced ADCC and/or complement activation.

14. A recombinant transformed or transfected host cell comprising a first and second vector, said first vector comprising a polynucleotide encoding a heavy chain of an antibody according to claim 6 and said second vector comprising a polynucleotide encoding a light chain of claim 6.

15. A pharmaceutical composition comprising an antigen binding protein of claim 1 and a pharmaceutically acceptable carrier.

16. A method of treating a human patient afflicted with immune system mediated inflammation such as psoriasis, inflammatory bowel disease, ulcerative colitis, crohns disease, rheumatoid arthritis, juvenile rheumatoid arthritis, systemic lupus erythematosus, neurodegenerative diseases, for example multiple sclerosis, neutrophil driven diseases, for example COPD, Wegeners vasculitis, cystic fibrosis, Sjogrens syndrome, chronic transplant rejection, type 1 diabetes graft versus host disease, asthma, allergic diseases atoptic dermatitis, eczematous dermatitis, allergic rhinitis, autoimmune diseases other including thyroiditis, spondyloarthropathy, ankylosing spondylitis, uveitis, polychonhtis or scleroderma which method comprises the step of administering a therapeutically effective amount of an antigen binding protein of claim 1.

17. (canceled)

18. (canceled)
